(19) United States
(12) Patent Application Publication
(10) Pub. No.: US 2024/0167054 A1
(43) Pub. Date: May 23, 2024

(54) METHOD OF PRODUCING A RECOMBINANT VIRUS PARTICLE

(71) Applicant: REGENXBIO INC., Rockville, MD (US)

(72) Inventors: Robert STADELMAN, New Market, MD (US); Donald STARTT, Bethesda, MD (US)

(21) Appl. No.: 18/257,820

(22) PCT Filed: Dec. 16, 2021
(86) PCT No.: PCT/US2021/063739

§ 371 Date: Jun. 15, 2023

Related U.S. Application Data
(60) Provisional application No. 63/126,405, filed on Dec. 16, 2020.

(51) Int. Cl. C12N 15/86; C12N 5/073

(52) U.S. Cl. CPC C12N 15/86; C12N 5/0603; C12N 2750/14143; C12N 2750/14152

(57) ABSTRACT

Provided herein are improved methods for producing recombinant virus particles. In some embodiments, a method for producing recombinant virus particles described herein comprises providing a suspension cell culture comprising a population of cells capable of producing the virus particle, transferring a first volume of poly nucleotide:transfection reagent complexes to the suspension culture to transfect the cells, and transferring a second volume of polynucleotide:transfection reagent complexes to the suspension culture, wherein the transferring of the first and second volume of polynucleotide:transfection reagent complexes are performed simultaneously or consecutively in any order. In some embodiments, the recombinant virus particles are recombinant AAV (rAAV) particles.

### CROSS-REFERENCE TO RELATED APPLICATIONS

This application is claims the benefit of U.S. Application No. 63/126,405, filed Dec. 16, 2020, which is incorporated herein by reference in its entirety.

### TECHNICAL FIELD

The present disclosure relates to a method of producing a recombinant virus particle in a large scale suspension cell culture comprising transfecting the cells in the culture.

### BACKGROUND

Recombinant adeno-associated virus (AAV)-based vectors are currently the most widely used gene therapy products in development. The preferred use of rAAV vector systems is due, in part, to the lack of disease associated with the wild-type virus, the ability of AAV to transduce non-dividing as well as dividing cells, and the resulting long-term robust transgene expression observed in clinical trials and that indicate great potential for delivery in gene therapy indications. Additionally, different naturally occurring and recombinant rAAV vector serotypes, specifically target different tissues, organs, and cells, and help evade any pre-existing immunity to the vector, thus expanding the therapeutic applications of AAV-based gene therapies. Before replication defective virus, for example, AAV based gene therapies can be more widely adopted for late clinical stage and commercial use, new methods for large scale production of recombinant virus particles need to be developed.

Thus, there is a need in the art to improve the productivity and yield of methods for the large scale production of rAAV particles.

### BRIEF SUMMARY

In one aspect, the disclosure provides a method of isolating recombinant adeno-associated virus (rAAV) genome using size exclusion chromatography. In some embodiments, the method comprises subjecting a composition comprising rAAV particles to a condition under which the rAAV particles are denatured prior to subjecting the composition comprising the denatured rAAV particles to size exclusion chromatography. In some embodiments, the mobile phase for the size exclusion chromatography comprises a salt, organic solvent, or detergent. In some embodiments, the mobile phase further comprises a buffering agent. In some embodiments, the rAAV comprises a capsid protein of the AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 serotype. In some embodiments, the rAAV comprises a capsid protein of the AAV8 or AAV9 serotype.

In a further aspect, the disclosure provides a method to characterize recombinant adeno-associated virus (rAAV) particles using size exclusion chromatography. Characterization of isolated rAAV particles includes but is not limited to determining vector genome size purity of a composition comprising isolated rAAV particles, assessing the folding or secondary structure of vector genomes inside the capsids, and determining vector genome titer (Vg) of a composition comprising isolated rAAV particles. In some embodiments, the mobile phase for the size exclusion chromatography comprises a salt, organic solvent, or detergent. In some embodiments, the mobile phase further comprises a buffering agent. In some embodiments, the rAAV particle comprises a capsid protein of the AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 serotype. In some embodiments, the rAAV comprises a capsid protein of the AAV8 or AAV9 serotype.

In some embodiments, a method disclosed herein is suitable for batch release, e.g. for batch release testing and/or lot release testing. In some embodiments, a method disclosed herein is performed as part of lot release testing.

In some embodiments, the disclosure provides:
[1.] A method of producing a recombinant virus particle, comprising
   a) providing a suspension cell culture of between about 200 liters and about 20,000 liters comprising a population of cells capable of producing the recombinant virus particle;
   b) admixing one or more polynucleotides with at least one transfection reagent to form a first mixture, incubating the mixture to form polynucleotide:transfection reagent complexes, and transferring the polynucleotide:transfection reagent complexes to the suspension culture to transfect the cells;
   c) admixing the one or more polynucleotides with the at least one transfection reagent to form a second mixture, incubating the mixture to form polynucleotide:transfection reagent complexes, and transferring the polynucleotide:transfection reagent complexes to the suspension culture to transfect the cells; and
   d) maintaining the cell culture comprising the transfected cells under conditions that allow the production of the recombinant virus particle,
   wherein
   (i) the one or more polynucleotide contain genes necessary for producing the recombinant virus particle;
   (ii) the admixing, incubating and transferring of step b) and step c) are each completed in less than about 60 minutes;
   (iii) the transferring in step b) and step c) is performed over a time period that is no longer than about 6 hours; and
   (iv) the transferring of step b) and step c) are performed simultaneously or consecutively in any order.
[2.] The method of [1], wherein step c) is repeated one more time.
[3.] The method of [1], wherein step c) is repeated one or more times.
[4.] The method of [1], wherein step c) is repeated 1, 2, 3, 4, 5, 6, 7 or 8 times.
[5.] The method of any one of [1] to [4], wherein the combined volume of the polynucleotide:transfection reagent complexes transferred to the suspension culture is between about 5% and about 20% of the volume of the suspension cell culture of step a).
[6.] The method of any one of [1] to [5], wherein the transferring of step c) starts between about 5 and about 60 minutes after completing the transferring of step b).
[7.] The method of any one of [1] to [5], wherein the transferring of step c) starts no more than about 60 minutes after completing the transferring of step b).
[8.] A method of increasing the production of a recombinant virus particle, comprising
   a) providing a suspension cell culture of between about 200 liters and about 20,000 liters comprising a population of cells capable of producing the recombinant virus particle;
   b) admixing one or more polynucleotides with at least one transfection reagent to form a first mixture, incubating the mixture to form polynucleotide:transfection reagent complexes, and transferring the polynucleotide:transfection reagent complexes to the suspension culture to transfect the cells;
   c) admixing the one or more polynucleotides with the at least one transfection reagent to form a second mixture, incubating the mixture to form polynucleotide:transfection reagent complexes, and transferring the polynucleotide:transfection reagent complexes to the suspension culture to transfect the cells; and
   d) maintaining the cell culture comprising the transfected cells under conditions that allow the production of the recombinant virus particle,
   wherein
   (i) the one or more polynucleotide contain genes necessary for producing the recombinant virus particle;
   (ii) the admixing, incubating and transferring of step b) and step c) are each completed in less than about 60 minutes;
   (iii) the transferring in step b) and step c) is performed over a time period that is no longer than about 6 hours; and
   (iv) the transferring of step b) and step c) are performed simultaneously or consecutively in any order.
[9.] The method of [8], wherein step c) is repeated one more time.
[10.] The method of [8], wherein step c) is repeated one or more times.
[11.] The method of [8], wherein step c) is repeated 1, 2, 3, 4, 5, 6, 7 or 8 times.
[12.] The method of any one of [8] to [11], wherein the combined volume of the polynucleotide:transfection reagent complexes transferred to the suspension culture is between about 5% and about 20% of the volume of the suspension cell culture of step a).
[13.] The method of any one of [8] to [12], wherein the transferring of step c) starts between about 5 and about 60 minutes after completing the transferring of step b).
[14.] The method of any one of [8] to [13], wherein the transferring of step c) starts no more than about 60 minutes after completing the transferring of step b).
[15.] The method of any one of [1] to [14], wherein the admixing one or more polynucleotides with at least one transfection reagent is performed by an inline mixer.
[16.] The method of any one of [1] to [15], wherein the admixing, incubating and transferring of step b) and step c) are each completed in less than about 30 minutes.
[17.] The method of any one of [1] to [15], wherein the admixing, incubating and transferring of step b) and step c) are each completed in less than about 35 minutes.
[18.] The method of any one of [1] to [17], wherein the incubating of step b) and step c) are each for about 10 to about 20 minutes.
[19.] The method of any one of [1] to [17], wherein the incubating of step b) and step c) are each for about 10 to about 15 minutes.
[20.] The method of any one of [1] to [19], wherein the at least one transfection reagent comprises a stable cationic polymer.
[21.] The method of any one of [1] to [20], wherein the at least one transfection reagent comprises PEI.
[22.] The method of any one of [1] to [21], wherein the cell culture has a volume between about 200 liters and about 5,000 liters.
[23.] The method of any one of [1] to [21], wherein the cell culture has a volume between about 200 liters and about 2,000 liters.
[24.] The method of any one of [1] to [21], wherein the cell culture has a volume between about 200 liters and about 1,000 liters.
[25.] The method of any one of [1] to [21], wherein the cell culture has a volume between about 200 liters and about 500 liters.
[26.] The method of any one of [1] to [21], wherein the cell culture has a volume of about 200 liters, about 300 liters, about 400 liters, about 500 liters, about 750 liters, about 1,000 liters, about 2,000 liters, about 3,000 liters, or about 5,000 liters.
[27.] The method of any one of [1] to [21], wherein the cell culture has a volume of about 500 liters.
[28.] The method of any one of [1] to [21], wherein the cell culture has a volume of about 1,000 liters.
[29.] The method of any one of [1] to [21], wherein the cell culture has a volume of about 2,000 liters.
[30.] The method of any one of [1] to [29], wherein the population of cells comprises a population of mammalian cells or insect cells.
[31.] The method of any one of [1] to [29], wherein the population of cells comprises a population of mammalian cells.
[21.] The method of any one of [1] to [29], wherein the population of cells comprises a population of HEK293 cells, HEK derived cells, CHO cells, CHO derived cells, HeLa cells, SF-9 cells, BHK cells, Vero cells, and/or PerC6 cells.
[33.] The method of any one of [1] to [29], wherein the population of cells comprises a population of HEK293 cells.
[34.] The method of any one of [1] to [33], wherein the suspension cell culture provided in step a) comprises between about 2×10E+6 and about 10E+7 viable cell/ml.
[35.] The method of any one of [1] to [34], wherein the cell culture is maintained under conditions that allow production of the recombinant virus particle for between about 2 days and about 10 days, between about 3 days and about 5 days, or between about 5 days and 14 days.
[36.] The method of any one of [1] to [34], wherein the cell culture is maintained for about 2 days, about 3 days, about 4 days, about 5 days, about 6 days, or about 7 days.
[37.] The method of any one of [1] to [34], wherein the cell culture is maintained for at least about 3 days.
[38.] The method of any one of [1] to [34], wherein the cell culture is maintained for about 3 days.
[39.] The method of any one of [1] to [34], wherein the cell culture is maintained for about 4 days.
[40.] The method of any one of [1] to [39], wherein the recombinant virus particle is a recombinant adeno-associated virus (rAAV) particle or a recombinant lentivirus particle.
[41.] The method of any one of [1] to [39], wherein the recombinant virus particle is an rAAV particle.
[42.] The method of [41], wherein the rAAV particle comprises a capsid protein of the AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 serotype.
[43.] The method of [41], wherein the rAAV particle comprises a capsid protein of the AAV8, AAV9, AAV.rh10, AAV.rh20, AAV.rh39, AAV.Rh74, AAV.RHM4-1, or AAV.hu37 serotype.
[44.] The method of [41], wherein the rAAV particles comprise a capsid protein of the AAV8 or AAV9 serotype.
[45.] The method of any one of [41] to [44], wherein the rAAV particle comprises a genome comprising a transgene.
[46.] The method of [45], wherein the transgene comprises a regulatory element operatively connected to a polynucleotide encoding a polypeptide.
[47.] The method of [46], wherein the regulatory element comprises one or more of an enhancer, promoter, and polyA region.
[48.] The method of [45] or [46], wherein the regulatory element and polynucleotide encoding a polypeptide are heterologous.
[49.] The method of any one of [45] to [48], wherein the transgene encodes an antibody or antigen-binding fragment thereof, fusion protein, Fc-fusion polypeptide, immunoadhesin, immunoglobulin, engineered protein, protein fragment or enzyme.
[50.] The method of any one of [45] to [48], wherein the transgene encodes an anti-VEGF Fab, iduronidase (IDUA), iduronate 2-sulfatase (IDS), low-density lipoprotein receptor (LDLR), tripeptidyl peptidase 1 (TPP1), or non-membrane associated splice variant of VEGF receptor 1 (sFlt-1).
[51.] The method of any one of [45] to [48], wherein the transgene encodes an gamma-sarcoglycan, Rab Escort Protein 1 (REP1/CHM), retinoid isomerohydrolase (RPE65), cyclic nucleotide gated channel alpha 3 (CNGA3), cyclic nucleotide gated channel beta 3 (CNGB3), aromatic L-amino acid decarboxylase (AADC), lysosome-associated membrane protein 2 isoform B (LAMP2B), Factor VIII, Factor IX, retinitis pigmentosa GTPase regulator (RPGR), retinoschisin (RS1), sarcoplasmic reticulum calcium ATPase (SERCA2a), aflibercept, battenin (CLN3), transmembrane ER protein (CLN6), glutamic acid decarboxylase (GAD), Glial cell line-derived neurotrophic factor (GDNF), aquaporin 1 (AQP1), dystrophin, microdystrophin, myotubularin 1 (MTM1), follistatin (FST), glucose-6-phosphatase (G6Pase), apolipoprotein A2 (APOA2), uridine diphosphate glucuronosyl transferase 1A1 (UGT1A1), arylsulfatase B (ARSB), N-acetyl-alpha-glucosaminidase (NAGLU), alpha-glucosidase (GAA), alpha-galactosidase (GLA), beta-galactosidase (GLB1), lipoprotein lipase (LPL), alpha 1-antitrypsin (AAT), phosphodiesterase 6B (PDE6B), ornithine carbamoyltransferase 90TC), survival motor neuron (SMN1), survival motor neuron (SMN2), neurturin (NRTN), Neurotrophin-3 (NT-3/NTF3), porphobilinogen deaminase (PBGD), nerve growth factor (NGF), mitochondrially encoded NADH:ubiquinone oxidoreductase core subunit 4 (MT-ND4), protective protein cathepsin A (PPCA), dysferlin, MER proto-oncogene, tyrosine kinase (MERTK), cystic fibrosis transmembrane conductance regulator (CFTR), or tumor necrosis factor receptor (TNFR)-immunoglobulin (IgG1) Fc fusion.
[52.] The method of any one of [41] to [51], wherein the one or more polynucleotide
   encode
   a) an rAAV genome to be packaged,
   b) adenovirus helper functions necessary for packaging,
   c) an AAV rep protein sufficient for packaging, and
   d) an AAV cap proteins sufficient for packaging.
[53.] The method of [52], wherein the one or more polynucleotide comprises a polynucleotide encoding the rAAV genome, a polynucleotide encoding the AAV rep protein and the AAV cap proteins, and a polynucleotide encoding the adenovirus helper functions.
[54.] The method of [52] or [53], wherein the adenovirus helper functions comprise at least one of an adenovirus E1a gene, E1b gene, E4 gene, E2a gene, and VA gene.
[55.] The method of any one of [412] to [54], further comprising recovering the rAAV particles.
[56.] The method of any one of [41] to [55], wherein the cell culture produces between about 5×10e+10 GC/ml and about 1×10e+12 GC/ml rAAV particles.
[57.] The method of any one of [41] to [56], wherein the cell culture produces at least about twice as many rAAV particles measured as GC/ml than a reference method comprising a single step of admixing, incubating and transferring the same volume of polynucleotide:transfection reagent complexes.
[58.] A composition comprising isolated rAAV particles that were produced by the method of any one of [41] to [57].

Still other features and advantages of the compositions and methods described herein will become more apparent from the following detailed description when read in conjunction with the accompanying drawings.

### BRIEF DESCRIPTION OF THE DRAWINGS

FIG. 1. Complex preparation process flow diagram for a single dose 50 L transient transfection.

FIG. 2. Complex preparation process flow diagram for a single dose 200 L transient transfection.

FIG. 3. Complex preparation process flow diagram for a single dose 500 L transient transfection.

FIG. 4. Bioreactor productivity of 50 L, 200 L, and 500 L reactors using single dose transfection.

FIG. 5. Increased complexing time results in larger transfection complexes.

FIG. 6. Complex preparation process flow diagram to test the effect of complexing time on transient transfection efficiency.

FIG. 7. Effect of complexing time on transient transfection efficiency.

FIG. 8. Complex preparation process flow diagram for 200 L split transient transfection.

FIG. 9. Productivity comparison between single dose and split transfections in 200 l bioreactor.

FIG. 10. Split transfection robustness.

FIG. 11. Complex preparation process flow diagram for 500 L split transient transfection.

FIG. 12. Bioreactor productivity of 500 L reactor using split transfection.

FIG. 13. Split transfection process flow diagram for 200 L scale down of 2,000 L process.

### DETAILED DESCRIPTION

Provided herein are methods for producing recombinant virus particles in a large scale culture, for example, suspension culture, comprising transferring more than one volumes of separately produced compositions comprising polynucleotide:transfection reagent complexes to the culture to transfect the cells, wherein the transferring of the more than one volumes of compositions is performed over a time period that is no longer than about 6 hours, and wherein the transferring of the more than one volumes of compositions are performed simultaneously or consecutively. In some embodiments, the transfection reagent comprises a stable cationic polymer, for example, PEI. In some embodiments, the large scale cell culture is between about 200 liters and about 20,000 liters, and the combined volume of the separately produced compositions comprising polynucleotide:transfection reagent complexes transferred to the culture is between about 5% and about 20% of the volume of the cell culture. In some embodiments, each volume of the separately produced compositions comprising polynucleotide:transfection reagent complexes is produced and transferred to the cell culture in no more than about 60 minutes, for example, in no more than about 30 minutes. In some embodiments, the combined volume of the separately produced compositions comprising polynucleotide:transfection reagent complexes is larger than the volume that can be produced in a single batch and transferred to the large scale culture in no more than about 60 minutes, for example, in no more than about 30 minutes. Without being bound by any particular theory, the methods disclosed herein provide increased productivity by allowing the transfer of a large total volume of compositions comprising polynucleotide:transfection reagent complexes to the cell culture wherein each component volume of compositions was produced and transferred to the cell culture in no more than about 60 minutes, for example, in no more than about 30 minutes. In some embodiments, each volume of the separately produced compositions comprising polynucleotide:transfection reagent complexes is produced and transferred to the cell culture in no more than 60 minutes, no more than 50 minutes, no more than 40 minutes, no more than 35 minutes, no more than 30 minutes, no more than 25 minutes or no more than 20 minutes. In some embodiments, each volume of the separately produced compositions comprising polynucleotide:transfection reagent complexes is produced and transferred to the cell culture in no more than 30 minutes. In some embodiments, the productivity of the method disclosed herein is at least about twice the productivity of a reference method comprising transferring the same total volume of compositions comprising polynucleotide:transfection reagent complexes produced in a single batch. In some embodiments, productivity is determined as viral particles per ml of culture at the time of harvest. In some embodiments, productivity is determined as the number of viral particles recovered from a unit volume, for example, 1 ml of the culture. In some embodiment the cell culture is a suspension cell culture. In some embodiments, the cell culture comprises adherent cells growing attached to microcarriers or macrocarriers in stirred bioreactors. In some embodiments, the cell culture is a suspension cell culture comprising HEK293 cells.

In some embodiments, the recombinant virus particles are recombinant adeno-associated virus (rAAV) particles. In some embodiments, the rAAV comprises a capsid protein of the AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 serotype. In some embodiments, the rAAV comprises a capsid protein of the AAV8 or AAV9 serotype.

Given the very high number of rAAV particles needed to prepare a single therapeutic unit dose, a two-fold increase in rAAV yield provides a significant reduction in the cost of goods per unit dose. Increased virus yield allows a corresponding reduction not only in the cost of consumables needed to produce rAAV particles, but also in the cost of capital expenditure in connection with building industrial virus purification facilities.

### Definitions

Unless defined otherwise, all technical and scientific terms used herein have the same meaning as commonly understood by one of ordinary skill in the art to which this disclosure is related. To facilitate an understanding of the disclosed methods, a number of terms and phrases are defined below.

“About” modifying, for example, the quantity of an ingredient in the compositions, concentration of an ingredient in the compositions, flow rate, rAAV particle yield, feed volume, salt concentration, and like values, and ranges thereof, employed in the methods provided herein, refers to variation in the numerical quantity that can occur, for example, through typical measuring and handling procedures used for making concentrates or use solutions; through inadvertent error in these procedures; through differences in the manufacture, source, or purity of the ingredients employed to make the compositions or carry out the methods; and like considerations. The term “about” also encompasses amounts that differ due to aging of a composition with a particular initial concentration or mixture. The term “about” also encompasses amounts that differ due to mixing or processing a composition with a particular initial concentration or mixture. Whether or not modified by the term “about” the claims include equivalents to the quantities. In some embodiments, the term “about” refers to ranges of approximately 10-20% greater than or less than the indicated number or range. In further embodiments, “about” refers to plus or minus 10% of the indicated number or range. For example, “about 10%” indicates a range of 9% to 11%.

“AAV” is an abbreviation for adeno-associated virus, and may be used to refer to the virus itself or modifications, derivatives, or pseudotypes thereof. The term covers all subtypes and both naturally occurring and recombinant forms, except where required otherwise. The abbreviation “rAAV” refers to recombinant adeno-associated virus. The term “AAV” includes AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), avian AAV, bovine AAV, canine AAV, equine AAV, primate AAV, non-primate AAV, and ovine AAV, and modifications, derivatives, or pseudotypes thereof. “Primate AAV” refers to AAV that infect primates, “non-primate AAV” refers to AAV that infect non-primate mammals, “bovine AAV” refers to AAV that infect bovine mammals, etc.

“Recombinant”, as applied to an AAV particle means that the AAV particle is the product of one or more procedures that result in an AAV particle construct that is distinct from an AAV particle in nature.

A recombinant adeno-associated virus particle “rAAV particle” refers to a viral particle composed of at least one AAV capsid protein and an encapsidated polynucleotide rAAV vector genome comprising a heterologous polynucleotide (i.e. a polynucleotide other than a wild-type AAV genome such as a transgene to be delivered to a mammalian cell). The rAAV particle may be of any AAV serotype, including any modification, derivative or pseudotype (e.g., AAV1, AAV2, AAV3, AAV4, AAV5, AAV6, AAV7, AAV8, AAV9, or AAV10, or derivatives/modifications/pseudotypes thereof). Such AAV serotypes and derivatives/modifications/pseudotypes, and methods of producing such serotypes/derivatives/modifications/pseudotypes are known in the art (see, e.g., Asokan et al., Mol. Ther. 20(4):699-708 (2012).

The rAAV particles of the disclosure may be of any serotype, or any combination of serotypes, (e.g., a population of rAAV particles that comprises two or more serotypes, e.g., comprising two or more of rAAV2, rAAV8, and rAAV9 particles). In some embodiments, the rAAV particles are rAAV1, rAAV2, rAAV3, rAAV4, rAAV5, rAAV6, rAAV7, rAAV8, rAAV9, rAAV10, or other rAAV particles, or combinations of two or more thereof). In some embodiments, the rAAV particles are rAAV8 or rAAV9 particles.

In some embodiments, the rAAV particles have an AAV capsid protein of a serotype selected from the group consisting of AAV1, AAV2, AAV3, AAV4, AAV5, AAV6, AAV7, AAV8, AAV9, AAV10, AAV11, AAV12, AAV13, AAV14, AAV15, AAV16 or a derivative, modification, or pseudotype thereof. In some embodiments, the rAAV particles have an AAV capsid protein of a serotype of AAV8, AAV9, or a derivative, modification, or pseudotype thereof.

The term “cell culture,” refers to cells grown in suspension or attached to microcarriers or macrocarriers, bioreactors, roller bottles, hyperstacks, microspheres, macrospheres, flasks and the like, as well as the components of the supernatant or suspension itself, including but not limited to rAAV particles, cells, cell debris, cellular contaminants, colloidal particles, biomolecules, host cell proteins, nucleic acids, and lipids, and flocculants. Large scale approaches, such as bioreactors, including suspension cultures and adherent cells growing attached to microcarriers or macrocarriers in stirred bioreactors, are encompassed by the term “cell culture.” Cell culture procedures for both large and small-scale production of virus particles or proteins are encompassed by the present disclosure. In some embodiments, the term “cell culture” refers to cells grown in suspension. In some embodiments, the term “cell culture” refers to adherent cells grown attached to microcarriers or macrocarriers in stirred bioreactors.

The terms “purifying”, “purification”, “separate”, “separating”, “separation”, “isolate”, “isolating”, or “isolation”, as used herein, refer to increasing the degree of purity of a target product, e.g., rAAV particles and rAAV genome from a sample comprising the target product and one or more impurities. Typically, the degree of purity of the target product is increased by removing (completely or partially) at least one impurity from the sample. In some embodiments, the degree of purity of the rAAV in a sample is increased by removing (completely or partially) one or more impurities from the sample by using a method described herein.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Where embodiments of the disclosure are described in terms of a Markush group or other grouping of alternatives, the disclosed method encompasses not only the entire group listed as a whole, but also each member of the group individually and all possible subgroups of the main group, and also the main group absent one or more of the group members. The disclosed methods also envisage the explicit exclusion of one or more of any of the group members in the disclosed methods.

Methods for Recombinant Virus Production

In some embodiments, the disclosure provides a method of producing a recombinant virus particle, comprising
a) providing a cell culture of between about 200 liters and about 20,000 liters comprising a population of cells capable of producing the recombinant virus particle;
b) admixing one or more polynucleotides with at least one transfection reagent to form a first mixture, incubating the mixture to form polynucleotide:transfection reagent complexes, and transferring the polynucleotide:transfection reagent complexes to the culture to transfect the cells;
c) admixing the one or more polynucleotides with the at least one transfection reagent to form a second mixture, incubating the mixture to form polynucleotide:transfection reagent complexes, and transferring the polynucleotide:transfection reagent complexes to the culture to transfect the cells; and
d) maintaining the cell culture comprising the transfected cells under conditions that allow the production of the recombinant virus particle,
   wherein the one or more polynucleotides contain genes necessary for producing the recombinant virus particle. In some embodiments, the one or more polynucleotides comprise one or more helper genes, rep genes, cap genes and transgenes (for example genes of interest or the rAAV genome to be packaged). In some embodiments, the combined volume of the polynucleotide:transfection reagent complexes transferred to the suspension culture is between about 5% and about 20% of the volume of the cell culture of step a). In some embodiments, the combined volume of the polynucleotide:transfection reagent complexes transferred to the suspension culture is between about 7% and about 15% of the volume of the cell culture of step a). In some embodiments, the combined volume of the polynucleotide:transfection reagent complexes transferred to the suspension culture is about 10% of the volume of the cell culture of step a). In some embodiments, the transferring of step c) starts before completing the transferring of step b). In some embodiments, the transferring of step c) starts immediately after completing the transferring of step b). In some embodiments, the transferring of step c) starts between about 5 minute and about 60 minutes after completing the transferring of step b). In some embodiments, the transferring of step c) starts no more than about 5 minutes after completing the transferring of step b). In some embodiments, the transferring of step c) starts no more than about 10 minutes after completing the transferring of step b). In some embodiments, the transferring of step c) starts no more than about 15 minutes after completing the transferring of step b). In some embodiments, the transferring of step c) starts no more than about 20 minutes after completing the transferring of step b). In some embodiments, the transferring of step c) starts no more than about 30 minutes after completing the transferring of step b). In some embodiments, the transferring of step c) starts no more than about 45 minutes after completing the transferring of step b). In some embodiments, the transferring of step c) starts no more than about 60 minutes after completing the transferring of step b). In some embodiments, the admixing, incubating and transferring of step b) and step c) are each completed in less than about 90 minutes, about 60 minutes, about 50 minutes, about 40 minutes, about 35 minutes, about 30 minutes, about 25 minutes, or about 20 minutes. In some embodiments, the admixing, incubating and transferring of step b) and step c) are each completed in less than about 60 minutes. In some embodiments, the admixing, incubating and transferring of step b) and step c) are each completed in less than about 50 minutes. In some embodiments, the admixing, incubating and transferring of step b) and step c) are each completed in less than about 40 minutes. In some embodiments, the admixing, incubating and transferring of step b) and step c) are each completed in less than about 35 minutes. In some embodiments, the admixing, incubating and transferring of step b) and step c) are each completed in less than about 30 minutes. In some embodiments, the admixing, incubating and transferring of step b) and step c) are each completed in less than about 25 minutes. In some embodiments, the admixing, incubating and transferring of step b) and step c) are each completed in less than about 20 minutes. In some embodiments, the incubating of step b) and step c) are for between about 5 minutes and about 20 minutes, between about 10 minutes and about 20 minutes, between about 5 minutes and about 15 minutes, between about 10 minutes and about 15 minutes, or between about 15 minutes and about 20 minutes. In some embodiments, the incubating of step b) and step c) are for between about 10 minutes and about 15 minutes. In some embodiments, the incubating of step b) and step c) are for about 5 minutes, about 10 minutes, about 12 minutes, about 13 minutes, about 14 minutes, about 15 minutes, about 16 minutes, about 17 minutes, about 18 minutes or about 20 minutes. In some embodiments, the incubating of step b) and step c) are for about 10 minutes. In some embodiments, the incubating of step b) and step c) are for about 12 minutes. In some embodiments, the incubating of step b) and step c) are for about 15 minutes. In some embodiments, the transferring in step b) and step c) is performed over a time period that is between about 1 hour and about 12 hours. In some embodiments, the transferring in step b) and step c) is performed over a time period that is between about 1 hour and about 8 hours. In some embodiments, the transferring in step b) and step c) is performed over a time period that is between about 1 hour and about 6 hours. In some embodiments, the transferring in step b) and step c) is performed over a time period that is between about 1 hour and about 5 hours. In some embodiments, the transferring in step b) and step c) is performed over a time period that is between about 1 hour and about 4 hours. In some embodiments, the transferring in step b) and step c) is performed over a time period that is between about 1 hour and about 3 hours. In some embodiments, the transferring in step b) and step c) is performed over a time period that is no longer than about 12 hours. In some embodiments, the transferring in step b) and step c) is performed over a time period that is no longer than about 9 hours. In some embodiments, the transferring in step b) and step c) is performed over a time period that is no longer than about 8 hours. In some embodiments, the transferring in step b) and step c) is performed over a time period that is no longer than about 7 hours. In some embodiments, the transferring in step b) and step c) is performed over a time period that is no longer than about 6 hours. In some embodiments, the transferring in step b) and step c) is performed over a time period that is no longer than about 5 hours. In some embodiments, the transferring in step b) and step c) is performed over a time period that is no longer than about 5 hours. In some embodiments, the transferring in step b) and step c) is performed over a time period that is no longer than about 3 hours. In some embodiments, the transferring in step b) and step c) is performed over a time period that is no longer than about 2 hours. In some embodiments, the transferring of step b) and step c) are performed simultaneously or consecutively in any order. In some embodiments, the transferring of step b) and step c) are performed consecutively in any order. In some embodiments, the admixing, incubating and transferring of step b) and step c) are performed in the same way. In some embodiments, the cell culture is a suspension culture. In some embodiments, the cell culture comprises HEK293 cells adapted for growth in suspension culture. In some embodiments, the cell culture has a volume of between about 400 liters and about 5,000 liters. In some embodiments, the cell culture has a volume of about 500 liters. In some embodiments, the cell culture has a volume of about 2,000 liters. In some embodiments, the transfection reagent comprises a cationic polymer. In some embodiments, the transfection reagent comprises PEI. In some embodiments, the recombinant virus particle is a recombinant adeno-associated virus (rAAV) particle. In some embodiments, the one or more polynucleotides comprise a mixture of three polynucleotides: one encoding the cap and rep genes, one encoding adenovirus helper functions necessary for packaging (e.g., adenovirus E1a gene, E1b gene, E4 gene, E2a gene, and VA gene), and one encoding the rAAV genome to be packaged. In some embodiments, the rAAV particles are AAV8 or AAV9 particles. In some embodiments, the rAAV particles have an AAV capsid protein of a serotype selected from the group consisting of AAV.rh8, AAV.rh10, AAV.rh20, AAV.rh39, AAV.Rh74, AAV.RHM4-1, AAV.hu37, AAV.PHB, and AAV.7m8. In some embodiments, the rAAV particles have an AAV capsid protein with high sequence homology to AAV8 or AAV9 such as, AAV.rh10, AAV.rh20, AAV.rh39, AAV.Rh74, AAV.RHM4-1, and AAV.hu37.

In some embodiments, the disclosure provides a method of producing a recombinant virus particle, comprising
a) providing a cell culture of between about 200 liters and about 20,000 liters comprising a population of cells capable of producing the recombinant virus particle;
b) admixing one or more polynucleotides with at least one transfection reagent to form a first mixture, incubating the mixture to form polynucleotide:transfection reagent complexes, and transferring the polynucleotide:transfection reagent complexes to the culture to transfect the cells;
c) admixing the one or more polynucleotides with the at least one transfection reagent to form a second mixture, incubating the mixture to form polynucleotide:transfection reagent complexes, and transferring the polynucleotide:transfection reagent complexes to the culture to transfect the cells; and
d) maintaining the cell culture comprising the transfected cells under conditions that allow the production of the recombinant virus particle,
   wherein the one or more polynucleotides contain genes necessary for producing the recombinant virus particle, and wherein step c) is repeated one or more times. In some embodiments, step c) is repeated one more time. In some embodiments, step c) is repeated 1, 2, 3, 4, 5, 6, 7, 8, 9 or 10 times. In some embodiments, step c) is repeated 2 times. In some embodiments, step c) is repeated 3 times. In some embodiments, step c) is repeated 4 times. In some embodiments, step c) is repeated 5 times. In some embodiments, step c) is repeated 6 times. In some embodiments, step c) is repeated 7 times. In some embodiments, step c) is repeated 8 times. In some embodiments, step c) is repeated 7 times. In some embodiments, step c) is repeated 9 times. In some embodiments, step c) is repeated 7 times. In some embodiments, step c) is repeated 10 times. In some embodiments, the combined volume of the polynucleotide:transfection reagent complexes transferred to the suspension culture is between about 5% and about 20% of the volume of the cell culture of step a). In some embodiments, the combined volume of the polynucleotide:transfection reagent complexes transferred to the suspension culture is between about 7% and about 15% of the volume of the cell culture of step a). In some embodiments, the combined volume of the polynucleotide:transfection reagent complexes transferred to the suspension culture is about 10% of the volume of the cell culture of step a). In some embodiments, the transferring of steps c) starts before completing the transferring of the preceding step. In some embodiments, the transferring of steps c) starts immediately after completing the transferring of the preceding step. In some embodiments, the transferring of steps c) starts between about 5 minute and about 60 minutes after completing the transferring of the preceding step. In some embodiments, the transferring of steps c) starts no more than about 5 minutes after completing the transferring of the preceding step. In some embodiments, the transferring of steps c) starts no more than about 10 minutes after completing the transferring of the preceding step. In some embodiments, the transferring of steps c) starts no more than about 15 minutes after completing the transferring of the preceding step. In some embodiments, the transferring of steps c) starts no more than about 20 minutes after completing the transferring of the preceding step. In some embodiments, the transferring of steps c) starts no more than about 30 minutes after completing the transferring of the preceding step. In some embodiments, the transferring of steps c) starts no more than about 45 minutes after completing the transferring of the preceding step. In some embodiments, the transferring of steps c) starts no more than about 60 minutes after completing the transferring of the preceding step. In some embodiments, the admixing, incubating and transferring of step b) and steps c) are each completed in less than about 90 minutes, about 60 minutes, about 50 minutes, about 40 minutes, about 35 minutes, about 30 minutes, about 25 minutes, or about 20 minutes. In some embodiments, the admixing, incubating and transferring of step b) and steps c) are each completed in less than about 60 minutes. In some embodiments, the admixing, incubating and transferring of step b) and steps c) are each completed in less than about 50 minutes. In some embodiments, the admixing, incubating and transferring of step b) and steps c) are each completed in less than about 40 minutes. In some embodiments, the admixing, incubating and transferring of step b) and steps c) are each completed in less than about 35 minutes. In some embodiments, the admixing, incubating and transferring of step b) and steps c) are each completed in less than about 30 minutes. In some embodiments, the admixing, incubating and transferring of step b) and steps c) are each completed in less than about 25 minutes. In some embodiments, the admixing, incubating and transferring of step b) and steps c) are each completed in less than about 20 minutes. In some embodiments, the incubating of step b) and step c) are for between about 5 minutes and about 20 minutes, between about 10 minutes and about 20 minutes, between about 5 minutes and about 15 minutes, between about 10 minutes and about 15 minutes, or between about 15 minutes and about 20 minutes. In some embodiments, the incubating of step b) and step c) are for between about 10 minutes and about 15 minutes. In some embodiments, the incubating of step b) and step c) are for about 5 minutes, about 10 minutes, about 12 minutes, about 13 minutes, about 14 minutes, about 15 minutes, about 16 minutes, about 17 minutes, about 18 minutes or about 20 minutes. In some embodiments, the incubating of step b) and step c) are for about 10 minutes. In some embodiments, the incubating of step b) and step c) are for about 12 minutes. In some embodiments, the incubating of step b) and step c) are for about 15 minutes. In some embodiments, the transferring in step b) and steps c) is performed over a time period that is between about 1 hour and about 12 hours. In some embodiments, the transferring in step b) and steps c) is performed over a time period that is between about 1 hour and about 8 hours. In some embodiments, the transferring in step b) and steps c) is performed over a time period that is between about 1 hour and about 6 hours. In some embodiments, the transferring in step b) and steps c) is performed over a time period that is between about 1 hour and about 5 hours. In some embodiments, the transferring in step b) and steps c) is performed over a time period that is between about 1 hour and about 4 hours. In some embodiments, the transferring in step b) and steps c) is performed over a time period that is between about 1 hour and about 3 hours. In some embodiments, the transferring in step b) and steps c) is performed over a time period that is no longer than about 12 hours. In some embodiments, the transferring in step b) and steps c) is performed over a time period that is no longer than about 9 hours. In some embodiments, the transferring in step b) and steps c) is performed over a time period that is no longer than about 8 hours. In some embodiments, the transferring in step b) and steps c) is performed over a time period that is no longer than about 7 hours. In some embodiments, the transferring in step b) and steps c) is performed over a time period that is no longer than about 6 hours. In some embodiments, the transferring in step b) and steps c) is performed over a time period that is no longer than about 5 hours. In some embodiments, the transferring in step b) and steps c) is performed over a time period that is no longer than about 5 hours. In some embodiments, the transferring in step b) and steps c) is performed over a time period that is no longer than about 3 hours. In some embodiments, the transferring in step b) and steps c) is performed over a time period that is no longer than about 2 hours. In some embodiments, the transferring of step b) and steps c) are performed simultaneously or consecutively in any order. In some embodiments, the transferring of step b) and steps c) are performed consecutively in any order. In some embodiments, the admixing, incubating and transferring of step b) and steps c) are performed in the same way. In some embodiments, the cell culture is a suspension culture. In some embodiments, the cell culture comprises HEK293 cells adapted for growth in suspension culture. In some embodiments, the cell culture has a volume of between about 400 liters and about 5,000 liters. In some embodiments, the cell culture has a volume of about 500 liters. In some embodiments, the cell culture has a volume of about 2,000 liters. In some embodiments, the transfection reagent comprises a cationic polymer. In some embodiments, the one or more polynucleotides comprise one or more helper genes, rep genes, cap genes and transgenes (for example genes of interest). In some embodiments, the transfection reagent comprises PEI. In some embodiments, the recombinant virus particle is a recombinant adeno-associated virus (rAAV) particle. In some embodiments, the one or more polynucleotides comprise a mixture of three polynucleotides: one encoding the cap and rep genes, one encoding adenovirus helper functions necessary for packaging (e.g., adenovirus E1a gene, E1b gene, E4 gene, E2a gene, and VA gene), and one encoding the rAAV genome to be packaged. In some embodiments, the rAAV particles are AAV8 or AAV9 particles. In some embodiments, the rAAV particles have an AAV capsid protein of a serotype selected from the group consisting of AAV.rh8, AAV.rh10, AAV.rh20, AAV.rh39, AAV.Rh74, AAV.RHM4-1, AAV.hu37, AAV.PHB, and AAV.7m8. In some embodiments, the rAAV particles have an AAV capsid protein with high sequence homology to AAV8 or AAV9 such as, AAV.rh10, AAV.rh20, AAV.rh39, AAV.Rh74, AAV.RHM4-1, and AAV.hu37.

In some embodiments, the disclosure provides a method of increasing the production of a recombinant virus particle, comprising
a) providing a cell culture of between about 200 liters and about 20,000 liters comprising a population of cells capable of producing the recombinant virus particle;
b) admixing one or more polynucleotides with at least one transfection reagent to form a first mixture, incubating the mixture to form polynucleotide:transfection reagent complexes, and transferring the polynucleotide:transfection reagent complexes to the culture to transfect the cells;
c) admixing the one or more polynucleotides with the at least one transfection reagent to form a second mixture, incubating the mixture to form polynucleotide:transfection reagent complexes, and transferring the polynucleotide:transfection reagent complexes to the culture to transfect the cells; and
d) maintaining the cell culture comprising the transfected cells under conditions that allow the production of the recombinant virus particle,
   wherein the one or more polynucleotides contain genes necessary for producing the recombinant virus particle. In some embodiments, the method produces at least about twice as many rAAV particles measured as GC/ml than a reference method comprising a single step of admixing, incubating and transferring the same volume of polynucleotide:transfection reagent complexes. In some embodiments, the method increases rAAV production by at least about 50%, at least about 75%, or at least about 100% compared to a reference method comprising a single step of admixing, incubating and transferring the same volume of polynucleotide:transfection reagent complexes. In some embodiments, a method disclosed herein increases rAAV production by at least about two-fold, at least about three-fold, or at least about five-fold compared to a reference method comprising a single step of admixing, incubating and transferring the same volume of polynucleotide:transfection reagent complexes. In some embodiments, the combined volume of the polynucleotide:transfection reagent complexes transferred to the suspension culture is between about 5% and about 20% of the volume of the cell culture of step a). In some embodiments, the combined volume of the polynucleotide:transfection reagent complexes transferred to the suspension culture is between about 7% and about 15% of the volume of the cell culture of step a). In some embodiments, the combined volume of the polynucleotide:transfection reagent complexes transferred to the suspension culture is about 10% of the volume of the cell culture of step a). In some embodiments, the transferring of step c) starts before completing the transferring of step b). In some embodiments, the transferring of step c) starts immediately after completing the transferring of step b). In some embodiments, the transferring of step c) starts between about 5 minute and about 60 minutes after completing the transferring of step b). In some embodiments, the transferring of step c) starts no more than about 5 minutes after completing the transferring of step b). In some embodiments, the transferring of step c) starts no more than about 10 minutes after completing the transferring of step b). In some embodiments, the transferring of step c) starts no more than about 15 minutes after completing the transferring of step b). In some embodiments, the transferring of step c) starts no more than about 20 minutes after completing the transferring of step b). In some embodiments, the transferring of step c) starts no more than about 30 minutes after completing the transferring of step b). In some embodiments, the transferring of step c) starts no more than about 45 minutes after completing the transferring of step b). In some embodiments, the transferring of step c) starts no more than about 60 minutes after completing the transferring of step b). In some embodiments, the admixing, incubating and transferring of step b) and step c) are each completed in less than about 90 minutes, about 60 minutes, about 50 minutes, about 40 minutes, about 35 minutes, about 30 minutes, about 25 minutes, or about 20 minutes. In some embodiments, the admixing, incubating and transferring of step b) and step c) are each completed in less than about 60 minutes. In some embodiments, the admixing, incubating and transferring of step b) and step c) are each completed in less than about 50 minutes. In some embodiments, the admixing, incubating and transferring of step b) and step c) are each completed in less than about 40 minutes. In some embodiments, the admixing, incubating and transferring of step b) and step c) are each completed in less than about 35 minutes. In some embodiments, the admixing, incubating and transferring of step b) and step c) are each completed in less than about 30 minutes. In some embodiments, the admixing, incubating and transferring of step b) and step c) are each completed in less than about 25 minutes. In some embodiments, the admixing, incubating and transferring of step b) and step c) are each completed in less than about 20 minutes. In some embodiments, the incubating of step b) and step c) are for between about 5 minutes and about 20 minutes, between about 10 minutes and about 20 minutes, between about 5 minutes and about 15 minutes, between about 10 minutes and about 15 minutes, or between about 15 minutes and about 20 minutes. In some embodiments, the incubating of step b) and step c) are for between about 10 minutes and about 15 minutes. In some embodiments, the incubating of step b) and step c) are for about 5 minutes, about 10 minutes, about 12 minutes, about 13 minutes, about 14 minutes, about 15 minutes, about 16 minutes, about 17 minutes, about 18 minutes or about 20 minutes. In some embodiments, the incubating of step b) and step c) are for about 10 minutes. In some embodiments, the incubating of step b) and step c) are for about 12 minutes. In some embodiments, the incubating of step b) and step c) are for about 15 minutes. In some embodiments, the transferring in step b) and step c) is performed over a time period that is between about 1 hour and about 12 hours. In some embodiments, the transferring in step b) and step c) is performed over a time period that is between about 1 hour and about 8 hours. In some embodiments, the transferring in step b) and step c) is performed over a time period that is between about 1 hour and about 6 hours. In some embodiments, the transferring in step b) and step c) is performed over a time period that is between about 1 hour and about 5 hours. In some embodiments, the transferring in step b) and step c) is performed over a time period that is between about 1 hour and about 4 hours. In some embodiments, the transferring in step b) and step c) is performed over a time period that is between about 1 hour and about 3 hours. In some embodiments, the transferring in step b) and step c) is performed over a time period that is no longer than about 12 hours. In some embodiments, the transferring in step b) and step c) is performed over a time period that is no longer than about 9 hours. In some embodiments, the transferring in step b) and step c) is performed over a time period that is no longer than about 8 hours. In some embodiments, the transferring in step b) and step c) is performed over a time period that is no longer than about 7 hours. In some embodiments, the transferring in step b) and step c) is performed over a time period that is no longer than about 6 hours. In some embodiments, the transferring in step b) and step c) is performed over a time period that is no longer than about 5 hours. In some embodiments, the transferring in step b) and step c) is performed over a time period that is no longer than about 5 hours. In some embodiments, the transferring in step b) and step c) is performed over a time period that is no longer than about 3 hours. In some embodiments, the transferring in step b) and step c) is performed over a time period that is no longer than about 2 hours. In some embodiments, the transferring of step b) and step c) are performed simultaneously or consecutively in any order. In some embodiments, the transferring of step b) and step c) are performed consecutively in any order. In some embodiments, the admixing, incubating and transferring of step b) and step c) are performed in the same way. In some embodiments, the cell culture is a suspension culture. In some embodiments, the cell culture comprises HEK293 cells adapted for growth in suspension culture. In some embodiments, the cell culture has a volume of between about 400 liters and about 5,000 liters. In some embodiments, the cell culture has a volume of about 500 liters. In some embodiments, the cell culture has a volume of about 2,000 liters. In some embodiments, the one or more polynucleotides comprise one or more helper genes, rep genes, cap genes and transgenes (for example genes of interest). In some embodiments, the transfection reagent comprises a cationic polymer. In some embodiments, the transfection reagent comprises PEI. In some embodiments, the recombinant virus particle is a recombinant adeno-associated virus (rAAV) particle. In some embodiments, the one or more polynucleotides comprise a mixture of three polynucleotides: one encoding the cap and rep genes, one encoding adenovirus helper functions necessary for packaging (e.g., adenovirus E1a gene, E1b gene, E4 gene, E2a gene, and VA gene), and one encoding the rAAV genome to be packaged. In some embodiments, the rAAV particles are AAV8 or AAV9 particles. In some embodiments, the rAAV particles have an AAV capsid protein of a serotype selected from the group consisting of AAV.rh8, AAV.rh10, AAV.rh20, AAV.rh39, AAV.Rh74, AAV.RHM4-1, AAV.hu37, AAV.PHB, and AAV.7m8. In some embodiments, the rAAV particles have an AAV capsid protein with high sequence homology to AAV8 or AAV9 such as, AAV.rh10, AAV.rh20, AAV.rh39, AAV.Rh74, AAV.RHM4-1, and AAV.hu37.

In some embodiments, the disclosure provides a method of increasing the production of a recombinant virus particle, comprising
a) providing a cell culture of between about 200 liters and about 20,000 liters comprising a population of cells capable of producing the recombinant virus particle;
b) admixing one or more polynucleotides with at least one transfection reagent to form a first mixture, incubating the mixture to form polynucleotide:transfection reagent complexes, and transferring the polynucleotide:transfection reagent complexes to the culture to transfect the cells;
c) admixing the one or more polynucleotides with the at least one transfection reagent to form a second mixture, incubating the mixture to form polynucleotide:transfection reagent complexes, and transferring the polynucleotide:transfection reagent complexes to the culture to transfect the cells; and
d) maintaining the cell culture comprising the transfected cells under conditions that allow the production of the recombinant virus particle,
   wherein the one or more polynucleotides contain genes necessary for producing the recombinant virus particle, and wherein step c) is repeated one or more times. In some embodiments, the method produces at least about twice as many rAAV particles measured as GC/ml than a reference method comprising a single step of admixing, incubating and transferring the same volume of polynucleotide:transfection reagent complexes. In some embodiments, the method increases rAAV production by at least about 50%, at least about 75%, or at least about 100% compared to a reference method comprising a single step of admixing, incubating and transferring the same volume of polynucleotide:transfection reagent complexes. In some embodiments, a method disclosed herein increases rAAV production by at least about two-fold, at least about three-fold, or at least about five-fold compared to a reference method comprising a single step of admixing, incubating and transferring the same volume of polynucleotide:transfection reagent complexes. In some embodiments, step c) is repeated one more time. In some embodiments, step c) is repeated 1, 2, 3, 4, 5, 6, 7, 8, 9 or 10 times. In some embodiments, step c) is repeated 2 times. In some embodiments, step c) is repeated 3 times. In some embodiments, step c) is repeated 4 times. In some embodiments, step c) is repeated 5 times. In some embodiments, step c) is repeated 6 times. In some embodiments, step c) is repeated 7 times. In some embodiments, step c) is repeated 8 times. In some embodiments, step c) is repeated 7 times. In some embodiments, step c) is repeated 9 times. In some embodiments, step c) is repeated 7 times. In some embodiments, step c) is repeated 10 times. In some embodiments, the combined volume of the polynucleotide:transfection reagent complexes transferred to the suspension culture is between about 5% and about 20% of the volume of the cell culture of step a). In some embodiments, the combined volume of the polynucleotide:transfection reagent complexes transferred to the suspension culture is between about 7% and about 15% of the volume of the cell culture of step a). In some embodiments, the combined volume of the polynucleotide:transfection reagent complexes transferred to the suspension culture is about 10% of the volume of the cell culture of step a). In some embodiments, the transferring of steps c) starts before completing the transferring of the preceding step. In some embodiments, the transferring of steps c) starts immediately after completing the transferring of the preceding step. In some embodiments, the transferring of steps c) starts between about 5 minute and about 60 minutes after completing the transferring of the preceding step. In some embodiments, the transferring of steps c) starts no more than about 5 minutes after completing the transferring of the preceding step. In some embodiments, the transferring of steps c) starts no more than about 10 minutes after completing the transferring of the preceding step. In some embodiments, the transferring of steps c) starts no more than about 15 minutes after completing the transferring of the preceding step. In some embodiments, the transferring of steps c) starts no more than about 20 minutes after completing the transferring of the preceding step. In some embodiments, the transferring of steps c) starts no more than about 30 minutes after completing the transferring of the preceding step. In some embodiments, the transferring of steps c) starts no more than about 45 minutes after completing the transferring of the preceding step. In some embodiments, the transferring of steps c) starts no more than about 60 minutes after completing the transferring of the preceding step. In some embodiments, the admixing, incubating and transferring of step b) and steps c) are each completed in less than about 90 minutes, about 60 minutes, about 50 minutes, about 40 minutes, about 35 minutes, about 30 minutes, about 25 minutes, or about 20 minutes. In some embodiments, the admixing, incubating and transferring of step b) and steps c) are each completed in less than about 60 minutes. In some embodiments, the admixing, incubating and transferring of step b) and steps c) are each completed in less than about 50 minutes. In some embodiments, the admixing, incubating and transferring of step b) and steps c) are each completed in less than about 40 minutes. In some embodiments, the admixing, incubating and transferring of step b) and steps c) are each completed in less than about 35 minutes. In some embodiments, the admixing, incubating and transferring of step b) and steps c) are each completed in less than about 30 minutes. In some embodiments, the admixing, incubating and transferring of step b) and steps c) are each completed in less than about 25 minutes. In some embodiments, the admixing, incubating and transferring of step b) and steps c) are each completed in less than about 20 minutes. In some embodiments, the incubating of step b) and step c) are for between about 5 minutes and about 20 minutes, between about 10 minutes and about 20 minutes, between about 5 minutes and about 15 minutes, between about 10 minutes and about 15 minutes, or between about 15 minutes and about 20 minutes. In some embodiments, the incubating of step b) and step c) are for between about 10 minutes and about 15 minutes. In some embodiments, the incubating of step b) and step c) are for about 5 minutes, about 10 minutes, about 12 minutes, about 13 minutes, about 14 minutes, about 15 minutes, about 16 minutes, about 17 minutes, about 18 minutes or about 20 minutes. In some embodiments, the incubating of step b) and step c) are for about 10 minutes. In some embodiments, the incubating of step b) and step c) are for about 12 minutes. In some embodiments, the incubating of step b) and step c) are for about 15 minutes. In some embodiments, the transferring in step b) and steps c) is performed over a time period that is between about 1 hour and about 12 hours. In some embodiments, the transferring in step b) and steps c) is performed over a time period that is between about 1 hour and about 8 hours. In some embodiments, the transferring in step b) and steps c) is performed over a time period that is between about 1 hour and about 6 hours. In some embodiments, the transferring in step b) and steps c) is performed over a time period that is between about 1 hour and about 5 hours. In some embodiments, the transferring in step b) and steps c) is performed over a time period that is between about 1 hour and about 4 hours. In some embodiments, the transferring in step b) and steps c) is performed over a time period that is between about 1 hour and about 3 hours. In some embodiments, the transferring in step b) and steps c) is performed over a time period that is no longer than about 12 hours. In some embodiments, the transferring in step b) and steps c) is performed over a time period that is no longer than about 9 hours. In some embodiments, the transferring in step b) and steps c) is performed over a time period that is no longer than about 8 hours. In some embodiments, the transferring in step b) and steps c) is performed over a time period that is no longer than about 7 hours. In some embodiments, the transferring in step b) and steps c) is performed over a time period that is no longer than about 6 hours. In some embodiments, the transferring in step b) and steps c) is performed over a time period that is no longer than about 5 hours. In some embodiments, the transferring in step b) and steps c) is performed over a time period that is no longer than about 5 hours. In some embodiments, the transferring in step b) and steps c) is performed over a time period that is no longer than about 3 hours. In some embodiments, the transferring in step b) and steps c) is performed over a time period that is no longer than about 2 hours. In some embodiments, the transferring of step b) and steps c) are performed simultaneously or consecutively in any order. In some embodiments, the transferring of step b) and steps c) are performed consecutively in any order. In some embodiments, the admixing, incubating and transferring of step b) and steps c) are performed in the same way. In some embodiments, the cell culture is a suspension culture. In some embodiments, the cell culture comprises HEK293 cells adapted for growth in suspension culture. In some embodiments, the cell culture has a volume of between about 400 liters and about 5,000 liters. In some embodiments, the cell culture has a volume of about 500 liters. In some embodiments, the cell culture has a volume of about 2,000 liters. In some embodiments, the transfection reagent comprises a cationic polymer. In some embodiments, the transfection reagent comprises PEI. In some embodiments, the recombinant virus particle is a recombinant adeno-associated virus (rAAV) particle. In some embodiments, the one or more polynucleotides comprise one or more helper genes, rep genes, cap genes and transgenes (for example genes of interest or the rAAV genome to be packaged). In some embodiments, the one or more polynucleotides comprise a mixture of three polynucleotides: one encoding the cap and rep genes, one encoding adenovirus helper functions necessary for packaging (e.g., adenovirus E1a gene, E1b gene, E4 gene, E2a gene, and VA gene), and one encoding the rAAV genome to be packaged. In some embodiments, the rAAV particles are AAV8 or AAV9 particles. In some embodiments, the rAAV particles have an AAV capsid protein of a serotype selected from the group consisting of AAV.rh8, AAV.rh10, AAV.rh20, AAV.rh39, AAV.Rh74, AAV.RHM4-1, AAV.hu37, AAV.PHB, and AAV.7m8. In some embodiments, the rAAV particles have an AAV capsid protein with high sequence homology to AAV8 or AAV9 such as, AAV.rh10, AAV.rh20, AAV.rh39, AAV.Rh74, AAV.RHM4-1, and AAV.hu37.

In some embodiments, the disclosure provides a method of producing a recombinant adeno-associated virus (rAAV) particle, comprising
a) providing a suspension cell culture of between about 200 liters and about 20,000 liters comprising a population of cells capable of producing rAAV;
b) admixing one or more polynucleotides with at least one transfection reagent to form a first mixture, incubating the mixture to form polynucleotide:transfection reagent complexes, and transferring the polynucleotide:transfection reagent complexes to the suspension cell culture to transfect the cells;
c) admixing the one or more polynucleotides with the at least one transfection reagent to form a second mixture, incubating the mixture to form polynucleotide:transfection reagent complexes, and transferring the polynucleotide:transfection reagent complexes to the suspension cell culture to transfect the cells; and
d) maintaining the suspension cell culture comprising the transfected cells under conditions that allow the production of the rAAV particle,
   wherein the transfection reagent comprises PEI, wherein the one or more polynucleotides contain genes necessary for producing the rAAV particle. In some embodiments, the disclosure provides a method of increasing the production of a recombinant adeno-associated virus (rAAV) particle, comprising
a) providing a suspension cell culture of between about 200 liters and about 20,000 liters comprising a population of cells capable of producing the rAAV particle;
b) admixing one or more polynucleotides with at least one transfection reagent to form a first mixture, incubating the mixture to form polynucleotide:transfection reagent complexes, and transferring the polynucleotide:transfection reagent complexes to the culture to transfect the cells;
c) admixing the one or more polynucleotides with the at least one transfection reagent to form a second mixture, incubating the mixture to form polynucleotide:transfection reagent complexes, and transferring the polynucleotide:transfection reagent complexes to the culture to transfect the cells; and
d) maintaining the suspension cell culture comprising the transfected cells under conditions that allow the production of the rAAV particle, wherein the transfection reagent comprises PEI, wherein the one or more polynucleotides contain genes necessary for producing the rAAV particle. In some embodiments, the method produces at least about twice as many rAAV particles measured as GC/ml than a reference method comprising a single step of admixing, incubating and transferring the same volume of polynucleotide:transfection reagent complexes. In some embodiments, the method increases rAAV production by at least about 50%, at least about 75%, or at least about 100% compared to a reference method comprising a single step of admixing, incubating and transferring the same volume of polynucleotide:transfection reagent complexes. In some embodiments, a method disclosed herein increases rAAV production by at least about two-fold, at least about three-fold, or at least about five-fold compared to a reference method comprising a single step of admixing, incubating and transferring the same volume of polynucleotide:transfection reagent complexes. In some embodiments, the disclosure provides a method of producing a recombinant adeno-associated virus (rAAV) particle, comprising a) providing a suspension cell culture of between about 200 liters and about 20,000 liters comprising a population of cells capable of producing the rAAV particle; b) admixing one or more polynucleotides with at least one transfection reagent to form a first mixture, incubating the mixture to form polynucleotide:transfection reagent complexes, and transferring the polynucleotide:transfection reagent complexes to the culture to transfect the cells; c) admixing the one or more polynucleotides with the at least one transfection reagent to form a second mixture, incubating the mixture to form polynucleotide:transfection reagent complexes, and transferring the polynucleotide:transfection reagent complexes to the culture to transfect the cells; and d) maintaining the suspension cell culture comprising the transfected cells under conditions that allow the production of the rAAV particle, wherein the transfection reagent comprises PEI, wherein the one or more polynucleotides contain genes necessary for producing the rAAV particle, and wherein step c) is repeated one or more times. In some embodiments, step c) is repeated one more time. In some embodiments, step c) is repeated 1, 2, 3, 4, 5, 6, 7, 8, 9 or 10 times. In some embodiments, step c) is repeated 2 times. In some embodiments, step c) is repeated 3 times. In some embodiments, step c) is repeated 4 times. In some embodiments, step c) is repeated 5 times. In some embodiments, step c) is repeated 6 times. In some embodiments, step c) is repeated 7 times. In some embodiments, step c) is repeated 8 times. In some embodiments, step c) is repeated 7 times. In some embodiments, step c) is repeated 9 times. In some embodiments, step c) is repeated 7 times. In some embodiments, step c) is repeated 10 times. In some embodiments, the disclosure provides a method of increasing the production of a recombinant adeno-associated virus (rAAV) particle, comprising a) providing a suspension cell culture of between about 200 liters and about 20,000 liters comprising a population of cells capable of producing the rAAV particle; b) admixing one or more polynucleotides with at least one transfection reagent to form a first mixture, incubating the mixture to form polynucleotide:transfection reagent complexes, and transferring the polynucleotide:transfection reagent complexes to the culture to transfect the cells; c) admixing the one or more polynucleotides with the at least one transfection reagent to form a second mixture, incubating the mixture to form polynucleotide:transfection reagent complexes, and transferring the polynucleotide:transfection reagent complexes to the culture to transfect the cells; and d) maintaining the suspension cell culture comprising the transfected cells under conditions that allow the production of the rAAV particle, wherein the transfection reagent comprises PEI, wherein the one or more polynucleotides contain genes necessary for producing the rAAV particle. In some embodiments, the method produces at least about twice as many rAAV particles measured as GC/ml than a reference method comprising a single step of admixing, incubating and transferring the same volume of polynucleotide:transfection reagent complexes. In some embodiments, the method increases rAAV production by at least about 50%, at least about 75%, or at least about 100% compared to a reference method comprising a single step of admixing, incubating and transferring the same volume of polynucleotide:transfection reagent complexes. In some embodiments, a method disclosed herein increases rAAV production by at least about two-fold, at least about three-fold, or at least about five-fold compared to a reference method comprising a single step of admixing, incubating and transferring the same volume of polynucleotide:transfection reagent complexes. In some embodiments, the combined volume of the polynucleotide:transfection reagent complexes transferred to the suspension cell culture is between about 5% and about 20% of the volume of the suspension cell culture of step a). In some embodiments, the combined volume of the polynucleotide:transfection reagent complexes transferred to the suspension cell culture is between about 5% and about 20% of the volume of the suspension cell culture of step a). In some embodiments, the combined volume of the polynucleotide:transfection reagent complexes transferred to the suspension cell culture is between about 7% and about 15% of the volume of the suspension cell culture of step a). In some embodiments, the combined volume of the polynucleotide:transfection reagent complexes transferred to the suspension cell culture is about 10% of the volume of the suspension cell culture of step a). In some embodiments, the transferring of step c) starts before completing the transferring of the preceding step. In some embodiments, the transferring of step c) starts immediately after completing the transferring of the preceding step. In some embodiments, the transferring of step c) starts between about 5 minute and about 60 minutes after completing the transferring of the preceding step. In some embodiments, the transferring of step c) starts no more than about 5 minutes after completing the transferring of the preceding step. In some embodiments, the transferring of step c) starts no more than about 10 minutes after completing the transferring of the preceding step. In some embodiments, the transferring of step c) starts no more than about 15 minutes after completing the transferring of the preceding step. In some embodiments, the transferring of step c) starts no more than about 20 minutes after completing the transferring of the preceding step. In some embodiments, the transferring of step c) starts no more than about 30 minutes after completing the transferring of the preceding step. In some embodiments, the transferring of step c) starts no more than about 45 minutes after completing the transferring of the preceding step. In some embodiments, the transferring of step c) starts no more than about 60 minutes after completing the transferring of step b). In some embodiments, the admixing, incubating and transferring of step b) and step c) are each completed in less than about 90 minutes, about 60 minutes, about 50 minutes, about 40 minutes, about 35 minutes, about 30 minutes, about 25 minutes, or about 20 minutes. In some embodiments, the admixing, incubating and transferring of step b) and step c) are each completed in less than about 60 minutes. In some embodiments, the admixing, incubating and transferring of step b) and step c) are each completed in less than about 50 minutes. In some embodiments, the admixing, incubating and transferring of step b) and step c) are each completed in less than about 40 minutes. In some embodiments, the admixing, incubating and transferring of step b) and step c) are each completed in less than about 35 minutes. In some embodiments, the admixing, incubating and transferring of step b) and step c) are each completed in less than about 30 minutes. In some embodiments, the admixing, incubating and transferring of step b) and step c) are each completed in less than about 25 minutes. In some embodiments, the admixing, incubating and transferring of step b) and step c) are each completed in less than about 20 minutes. In some embodiments, the incubating of step b) and step c) are for between about 5 minutes and about 20 minutes, between about 10 minutes and about 20 minutes, between about 5 minutes and about 15 minutes, between about 10 minutes and about 15 minutes, or between about 15 minutes and about 20 minutes. In some embodiments, the incubating of step b) and step c) are for between about 10 minutes and about 15 minutes. In some embodiments, the incubating of step b) and step c) are for about 5 minutes, about 10 minutes, about 12 minutes, about 13 minutes, about 14 minutes, about 15 minutes, about 16 minutes, about 17 minutes, about 18 minutes or about 20 minutes. In some embodiments, the incubating of step b) and step c) are for about 10 minutes. In some embodiments, the incubating of step b) and step c) are for about 12 minutes. In some embodiments, the incubating of step b) and step c) are for about 15 minutes. In some embodiments, the transferring in step b) and step c) is performed over a time period that is between about 1 hour and about 12 hours. In some embodiments, the transferring in step b) and step c) is performed over a time period that is between about 1 hour and about 8 hours. In some embodiments, the transferring in step b) and step c) is performed over a time period that is between about 1 hour and about 6 hours. In some embodiments, the transferring in step b) and step c) is performed over a time period that is between about 1 hour and about 5 hours. In some embodiments, the transferring in step b) and step c) is performed over a time period that is between about 1 hour and about 4 hours. In some embodiments, the transferring in step b) and step c) is performed over a time period that is between about 1 hour and about 3 hours. In some embodiments, the transferring in step b) and step c) is performed over a time period that is no longer than about 12 hours. In some embodiments, the transferring in step b) and step c) is performed over a time period that is no longer than about 9 hours. In some embodiments, the transferring in step b) and step c) is performed over a time period that is no longer than about 8 hours. In some embodiments, the transferring in step b) and step c) is performed over a time period that is no longer than about 7 hours. In some embodiments, the transferring in step b) and step c) is performed over a time period that is no longer than about 6 hours. In some embodiments, the transferring in step b) and step c) is performed over a time period that is no longer than about 5 hours. In some embodiments, the transferring in step b) and step c) is performed over a time period that is no longer than about 5 hours. In some embodiments, the transferring in step b) and step c) is performed over a time period that is no longer than about 3 hours. In some embodiments, the transferring in step b) and step c) is performed over a time period that is no longer than about 2 hours. In some embodiments, the transferring of step b) and step c) are performed simultaneously or consecutively in any order. In some embodiments, the transferring of step b) and step c) are performed consecutively in any order. In some embodiments, the admixing, incubating and transferring of step b) and step c) are performed in the same way. In some embodiments, the suspension cell culture comprises HEK293 cells adapted for growth in suspension cell culture. In some embodiments, the suspension cell culture has a volume of between about 400 liters and about 10,000 liters. In some embodiments, the cell culture has a volume of about 500 liters. In some embodiments, the cell culture has a volume of about 2,000 liters. In some embodiments, the one or more polynucleotides comprise one or more helper genes, rep genes, cap genes and transgenes (for example genes of interest or the rAAV genome to be packaged). In some embodiments, the one or more polynucleotides comprise a mixture of three polynucleotides: one encoding the cap and rep genes, one encoding adenovirus helper functions necessary for packaging (e.g., adenovirus E1a gene, E1b gene, E4 gene, E2a gene, and VA gene), and one encoding the rAAV genome to be packaged. In some embodiments, the rAAV particles are AAV8 or AAV9 particles. In some embodiments, the rAAV particles have an AAV capsid protein of a serotype selected from the group consisting of AAV.rh8, AAV.rh10, AAV.rh20, AAV.rh39, AAV.Rh74, AAV.RHM4-1, AAV.hu37, AAV.PHB, and AAV.7m8. In some embodiments, the rAAV particles have an AAV capsid protein with high sequence homology to AAV8 or AAV9 such as, AAV.rh10, AAV.rh20, AAV.rh39, AAV.Rh74, AAV.RHM4-1, and AAV.hu37.

Transfection based recombinant virus particle production systems are known to the skilled artisan. See, e.g., Reiser et al., Gene Ther 7(11):910-3 (2000); Dull et al., J Virol. 72(11): 8463-8471 (1998); Hoffmann et al., PNAS 97 (11) 6108-6113 (2000); Milian et al., Vaccine 35(26): 3423-3430 (2017), each of which is incorporated herein by reference in its entirety. A method disclosed herein can be used to produce a recombinant virus particle in a transfection based production system. In some embodiments, the recombinant viral particle is a recombinant Dengue virus, a recombinant Ebola virus, a recombinant human papillomavirus (HPV), a recombinant human immunodeficiency virus (HIV), a recombinant adeno-associated virus (AAV), a recombinant lentivirus, a recombinant influenza virus, a recombinant vesicular stomatitis virus (VSV), a recombinant poliovirus, a recombinant adenovirus, a recombinant retrovirus, a recombinant vaccinia, a recombinant reovirus, a recombinant measles, a recombinant Newcastle disease virus (NDV), a recombinant herpes zoster virus (HZV), a recombinant herpes simplex virus (HSV), or a recombinant baculovirus. In some embodiments, the recombinant viral particle is a recombinant adeno-associated virus (AAV), a recombinant lentivirus, or a recombinant influenza virus. In some embodiments, the recombinant viral particle is a recombinant lentivirus. In some embodiments, the recombinant viral particle is a recombinant influenza virus. In some embodiments, the recombinant viral particle is a recombinant baculovirus. In some embodiments, the recombinant viral particle is a recombinant adeno-associated virus (AAV). In some embodiments, the rAAV particles are AAV8 or AAV9 particles. In some embodiments, the rAAV particles have an AAV capsid protein of a serotype selected from the group consisting of AAV.rh8, AAV.rh10, AAV.rh20, AAV.rh39, AAV.Rh74, AAV.RHM4-1, AAV.hu37, AAV.PHB, and AAV.7m8. In some embodiments, the rAAV particles have an AAV capsid protein with high sequence homology to AAV8 or AAV9 such as, AAV.rh10, AAV.rh20, AAV.rh39, AAV.Rh74, AAV.RHM4-1, and AAV.hu37.

Any suitable transfection reagent known in the art for transfecting a cell may be used for the production of recombinant virus particles (e.g., rAAV particles) according to a method disclosed herein. In some embodiments, the cell is a HEK293 cell, such as a HEK293 cell adapted for suspension culture. In some embodiments, a method disclosed herein comprises transfecting a cell using a chemical based transfection method. In some embodiments, a method disclosed herein comprises transfecting a cell using a cationic organic carrier. See, e.g., Gigante et al., MedChemComm 10(10): 1692-1718 (2019); Damen et al. MedChemComm 9(9): 1404-1425 (2018), each of which is incorporated herein by reference in its entirety. In some embodiments, the cationic organic carrier comprises a lipid, for example, DOTMA, DOTAP, helper lipids (Dope, cholesterol), and combinations thereof. In some embodiments, the cationic organic carrier comprises a multivalent cationic lipid, for example, DOSPA, DOGS, and mixtures thereof. In some embodiments, the cationic organic carrier comprises bipolar lipids, or bolaamphiphiles (bolas). In some embodiments, the cationic organic carrier comprises bioreducible and/or dimerizable lipids. In some embodiments, the cationic organic carrier comprises gemini surfactants. In some embodiments, the cationic organic carrier comprises Lipofectin™ Transfectam™, Lipofectamine™, Lipofectamine 2000™, or Lipofectamin PLUS 2000™ In some embodiments, the cationic organic carrier comprises a polymer, for example, poly(L-Lysine) (PLL), polyethylenimine (PEI), polysaccharides (chitosan, dextran, cyclodextrine (CD)), Poly[2-(dimethylamino) ethyl methacrylate] (PDMAEMA), and dendrimers (polyamidoamine (PAMAM), poly(propylene imine) (PPI)). In some embodiments, the cationic organic carrier comprises a peptide, for example, peptides rich in basic amino-acids (CWLis), cell penetrating peptides (CPPs) (Arg-rich peptides (octaarginine, TAT)), nuclear localization signals (NLS) (SV40) and targeting (RGD). In some embodiments, the cationic organic carrier comprises a polymers (e.g., PEI) combined with a cationic liposome. Paris et al., Molecules 25(14): 3277 (2020), which is incorporated herein by reference in its entirety. In some embodiments, the transfection reagent comprises calcium phosphate, highly branched organic compounds (dendrimers), cationic polymers (e.g., DEAE dextran or polyethylenimine (PEI)), lipofection.

In some embodiments, the transfection reagent comprises poly(L-Lysine) (PLL), polyethylenimine (PEI), linear PEI, branched PEI, dextran, cyclodextrine (CD), Poly[2-(dimethylamino) ethyl methacrylate] (PDMAEMA), polyamidoamine (PAMAM), poly(propylene imine) (PPI)), or mixtures thereof. In some embodiments, the transfection reagent comprises polyethylenimine (PEI), linear PEI, branched PEI, or mixtures thereof. In some embodiments, the transfection reagent comprises polyethylenimine (PEI). In some embodiments, the transfection reagent comprises linear PEI. In some embodiments, the transfection reagent comprises branched PEI. In some embodiments, the transfection reagent comprises polyethylenimine (PEI) having a molecular weight between about 5 and about 25 kDa. In some embodiments, the transfection reagent comprises PEGylated polyethylenimine (PEI). In some embodiments, the transfection reagent comprises modified polyethylenimine (PEI) to which hydrophobic moieties such cholesterol, choline, alkyl groups and some amino acids were attached.

The composition polynucleotide:transfection reagent complexes can be prepared by any method known to one of skill in the art. In some embodiments, admixing one or more polynucleotides with at least one transfection reagent comprises diluting each of the transfection reagent and the one or more polynucleotides into a sterile liquid, for example, tissue culture media, and mixing the diluted transfection reagent and diluted one or more polynucleotides. In some embodiments, the mixing comprises transferring the diluted one or more polynucleotides and the diluted at least one transfection reagent from two separate containers into a new container. In some embodiments, transferring the diluted one or more polynucleotides and the diluted at least one transfection reagent into a new container is performed at a rate of about 500 ml/min, about 1 liter/min, about 2 liters/min, about 3 liters/min, about 4 liters/min, about 5 liters/min, about 6 liters/min, about 7 liters/min, about 8 liters/min, about 9 liters/min, or about 10 liters/min. In some embodiments, the transferring is performed at a rate of about 3 liters/min. In some embodiments, the transferring is performed at a rate of about 4 liters/min. In some embodiments, the transferring is performed at a rate of about 5 liters/min. In some embodiments, the transferring is performed at a rate of about 6 liters/min. In some embodiments, mixing the diluted one or more polynucleotides with the diluted at least one transfection reagent is performed by an inline mixer. In some embodiments, the inline mixer is a low shear inline mixer. In some embodiments, the inline mixer is a static inline mixer. Inline mixers suitable for mixing polynucleotides with transfection reagent are known in the art and can be obtained, for example, from Sartorius (Flexsafe® Pro Mixer), Analytical Scientific Instruments US, Inc. (HYPERSHEAR™), Fluitec or STRIKO. One of skill understands that the dilution and mixing is conducted so as to produce a composition comprising the transfection reagent and polynucleotides at a desired ratio and concentration. In some embodiments, the dilution and mixing of the at least one transfection reagent and one or more polynucleotides produces a composition comprising the transfection reagent and the polynucleotide at a weight ratio between about 1:5 and 5:1. In some embodiments, the weight ratio of the transfection reagent and polynucleotide is between about 1:3 and 3:1. In some embodiments, the weight ratio of the transfection reagent and polynucleotide is between about 1:3 and 1:1. In some embodiments, the weight ratio of the transfection reagent and polynucleotide is between about 1:2 and 1:1.5. In some embodiments, the weight ratio of the transfection reagent and polynucleotide is about 1:5, 1:4, 1:3, 1:2.5, 1:2, 1:1.75, 1:1.5, 1:1.25, 1:1, 1.25:1, 1.5:1, 1.75:1, 2:1, 2.5:1, 3:1, 4:1, or 5:1. In some embodiments, the weight ratio of the transfection reagent and polynucleotide is about 1:2. In some embodiments, the weight ratio of the transfection reagent and polynucleotide is about 1:1.75. In some embodiments, the weight ratio of the transfection reagent and polynucleotide is about 1:1.5. In some embodiments, the weight ratio of the transfection reagent and polynucleotide is about 1:1.25. In some embodiments, the weight ratio of the transfection reagent and polynucleotide is about 1:1. In some embodiments, the weight ratio of the transfection reagent and polynucleotide is about 1.25:1. In some embodiments, the weight ratio of the transfection reagent and polynucleotide is about 1.5:1. In some embodiments, the weight ratio of the transfection reagent and polynucleotide is about 1.75:1. In some embodiments, the weight ratio of the transfection reagent and polynucleotide is about 2:1. In some embodiments, the composition comprises between about 1 μg and about 20 μg of the one or more polynucleotides. In some embodiments, the one or more polynucleotides comprise 3 plasmids. In some embodiments, the one or more polynucleotides comprise 2 plasmids. In some embodiments, the one or more polynucleotides comprise 1 plasmid. In some embodiments, the recombinant virus is a recombinant AAV and the one or more polynucleotides comprise a mixture of three polynucleotides: one encoding the cap and rep genes, one encoding adenovirus helper functions necessary for packaging (e.g., adenovirus E1a gene, E1b gene, E4 gene, E2a gene, and VA gene), and one encoding the rAAV genome to be packaged. In some embodiments, the rAAV particles are AAV8 or AAV9 particles. In some embodiments, the rAAV particles have an AAV capsid protein of a serotype selected from the group consisting of AAV.rh8, AAV.rh10, AAV.rh20, AAV.rh39, AAV.Rh74, AAV.RHM4-1, AAV.hu37, AAV.PHB, and AAV.7m8. In some embodiments, the rAAV particles have an AAV capsid protein with high sequence homology to AAV8 or AAV9 such as, AAV.rh10, AAV.rh20, AAV.rh39, AAV.Rh74, AAV.RHM4-1, and AAV.hu37. In some embodiments, the transfection reagent is PEI.

In some embodiments, the composition comprising the transfection reagent and one or more polynucleotides is incubated to allow the formation of polynucleotide:transfection reagent complexes. In some embodiments, the incubation is at room temperature. In some embodiments, the incubation comprises shaking the composition, for example, on a shaker at between about 100 and about 200 rpm. In some embodiments, the incubation is for between about 5 minutes and about 20 minutes, between about 10 minutes and about 20 minutes, between about 5 minutes and about 15 minutes, between about 10 minutes and about 15 minutes, or between about 15 minutes and about 20 minutes. In some embodiments, the incubation is for between about 5 minutes and about 20 minutes. In some embodiments, the incubation is for about 10 to about 15 minutes. In some embodiments, the incubation is for about 5 minutes, about 10 minutes, about 12 minutes, about 13 minutes, about 14 minutes, about 15 minutes, about 16 minutes, about 17 minutes, about 18 minutes or about 20 minutes. In some embodiments, the incubation is for about 11 minutes. In some embodiments, the incubation is for about 12 minutes. In some embodiments, the incubation is for about 13 minutes. In some embodiments, the incubation is for about 14 minutes. In some embodiments, the incubation is for about 15 minutes. In some embodiments, the incubation is for no longer than 15 minutes. In some embodiments, the incubation is for no longer than 10 minutes. In some embodiments, the incubation is for about 5 minutes, about 10 minutes, or about 15 minutes. In some embodiments, the incubation is for about 10 minutes. In some embodiments, the length of the incubation is such that the admixing, incubating and transferring is completed in in less than about 90 minutes, about 60 minutes, about 50 minutes, about 40 minutes, about 35 minutes, about 30 minutes, about 25 minutes, or about 20 minutes. In some embodiments, the admixing, incubating and transferring is completed in less than about 30 minutes. In some embodiments, the one or more polynucleotides contain genes necessary for producing of recombinant AAV particles. In some embodiments, the transfection reagent comprises PEI.

Methods for transferring the polynucleotide:transfection reagent complexes to the suspension culture are known to the skilled artisan. In some embodiments, the transferring is performed using a peristaltic pump. In some embodiments, the transferring is performed at a rate of between about 100 ml/min and about 10 liters/minute. In some embodiments, the transferring is performed at a rate of about 500 ml/min, about 1 liter/min, about 2 liters/min, about 3 liters/min, about 4 liters/min, about 5 liters/min, about 6 liters/min, about 7 liters/min, about 8 liters/min, about 9 liters/min, or about 10 liters/min. In some embodiments, the transferring is performed at a rate of about 3 liters/min. In some embodiments, the transferring is performed at a rate of about 4 liters/min. In some embodiments, the transferring is performed at a rate of about 5 liters/min. In some embodiments, the transferring is performed at a rate of about 6 liters/min. In some embodiments, the transferring is performed at a rate of about 7 liters/min. In some embodiments, the transferring is performed at a rate of about 8 liters/min. In some embodiments, the transferring is performed at a rate of about 9 liters/min. In some embodiments, the transferring is performed at a rate of about 10 liters/min. In some embodiments, the rate of transfer is set such that the admixing, incubating and transferring is completed in in less than about 90 minutes, about 60 minutes, about 50 minutes, about 40 minutes, about 35 minutes, about 30 minutes, about 25 minutes, or about 20 minutes. In some embodiments, the admixing, incubating and transferring is completed in less than about 30 minutes. In some embodiments, the admixing, incubating and transferring is completed in less than about 35 minutes. In some embodiments, the polynucleotides contain genes necessary for producing of recombinant AAV particles. In some embodiments, the transfection reagent comprises PEI. In some embodiments, the culture comprises HEK293 cells, such as HEK293 cells adapted for suspension culture.

In some embodiments, the separately produced polynucleotide:transfection reagent complexes are produced using the same process. In some embodiments, the separate admixing one or more polynucleotides with at least one transfection reagent to form a first mixture, incubating the mixture to form polynucleotide:transfection reagent complexes, and transferring the polynucleotide:transfection reagent complexes to the suspension culture to transfect the cells uses the same process. In some embodiments, the separate transfers of polynucleotide:transfection reagent complexes to the suspension culture comprise the transfer of the same volume of polynucleotide:transfection reagent complexes. In some embodiments, the separate transfers of polynucleotide:transfection reagent complexes to the suspension culture comprise the transfer of different volumes of polynucleotide:transfection reagent complexes. In some embodiments, the one or more polynucleotides contain genes necessary for producing of recombinant AAV particles. In some embodiments, the transfection reagent comprises PEI. In some embodiments, the culture comprises HEK293 cells, such as HEK293 cells adapted for suspension culture.

In some embodiments, the combined volume of the polynucleotide:transfection reagent complexes transferred to the cell culture is between about 5% and about 20% of the volume of the cell culture comprising a population of cells capable of producing the recombinant virus particle (e.g., rAAV particle). In some embodiments, the combined volume of the polynucleotide:transfection reagent complexes transferred is between about 7% and about 15% of the volume of the cell culture. In some embodiments, the combined volume of the polynucleotide:transfection reagent complexes transferred is about 10% of the volume of the cell culture. In some embodiments, the one or more polynucleotides contain genes necessary for producing of recombinant AAV particles. In some embodiments, the transfection reagent comprises PEI. In some embodiments, the culture comprises HEK293 cells, such as HEK293 cells adapted for suspension culture.

In some embodiments, the combined volume of the polynucleotide:transfection reagent complexes transferred to the cell culture comprise between about 0.1p g of the one or more polynucleotides/10E+6 viable cell/ml and about 5 μg of the one or more polynucleotides/10E+6 viable cell/ml. In some embodiments, the combined volume of the polynucleotide:transfection reagent complexes transferred to the cell culture comprise between about 0.2 μg of the one or more polynucleotides/10E+6 viable cell/ml and about 2 μg of the one or more polynucleotides/10E+6 viable cell/ml. In some embodiments, the combined volume of the polynucleotide:transfection reagent complexes transferred to the cell culture comprise about 0.2, 0.3, 0.4, 0.5, 0.6, 0.7, 0.8, 0.9 or 1 μg of the one or more polynucleotides/10E+6 viable cell/ml. In some embodiments, the one or more polynucleotides contain genes necessary for producing of recombinant AAV particles. In some embodiments, the transfection reagent comprises PEI. In some embodiments, the culture comprises HEK293 cells, such as HEK293 cells adapted for suspension culture.

In some embodiments, the cell culture has a volume of between about 400 liters and about 20,000 liters. In some embodiments, the cell culture has a volume of between about 500 liters and about 20,000 liters. In some embodiments, the cell culture has a volume of between about 700 liters and about 20,000 liters. In some embodiments, the cell culture has a volume of between about 1,000 liters and about 20,000 liters. In some embodiments, the cell culture has a volume of between about 400 liters and about 10,000 liters. In some embodiments, the cell culture has a volume of between about 500 liters and about 10,000 liters. In some embodiments, the cell culture has a volume of between about 700 liters and about 10,000 liters. In some embodiments, the cell culture has a volume of between about 1,000 liters and about 10,000 liters. In some embodiments, the cell culture has a volume of between about 400 liters and about 5,000 liters. In some embodiments, the cell culture has a volume of between about 500 liters and about 5,000 liters. In some embodiments, the cell culture has a volume of between about 700 liters and about 5,000 liters. In some embodiments, the cell culture has a volume of between about 1,000 liters and about 5,000 liters. In some embodiments, the cell culture volume referenced herein is the final bioreactor/vessel capacity as described in Table 1. In some embodiments, the culture comprises HEK293 cells, such as HEK293 cells adapted for suspension culture.

In some embodiments, the cell culture has a volume of between about 200 liters and about 5,000 liters. In some embodiments, the cell culture has a volume of between about 200 liters and about 2,000 liters. In some embodiments, the cell culture has a volume of between about 200 liters and about 1,000 liters. In some embodiments, the cell culture has a volume of between about 200 liters and about 500 liters. In some embodiments, the cell culture volume referenced herein is the final bioreactor/vessel capacity as described in Table 1. In some embodiments, the culture comprises HEK293 cells, such as HEK293 cells adapted for suspension culture.

In some embodiments, the cell culture has a volume of about 200 liters. In some embodiments, the cell culture has a volume of about 300 liters. In some embodiments, the cell culture has a volume of about 400 liters. In some embodiments, the cell culture has a volume of about 500 liters. In some embodiments, the cell culture has a volume of about 750 liters. In some embodiments, the cell culture has a volume of about 1,000 liters. In some embodiments, the cell culture has a volume of about 2,000 liters. In some embodiments, the cell culture has a volume of about 3,000 liters. In some embodiments, the cell culture has a volume of about 5,000 liters. In some embodiments, the culture comprises HEK293 cells, such as HEK293 cells adapted for suspension culture.

In some embodiments, a method described herein comprises transferring 2 separately produced about 20-liter volumes, such as about 21 liters of polynucleotide:transfection reagent complexes to a cell culture of about 400 liters. In some embodiments, a method described herein comprises transferring 3 separately produced about 20-liter volumes of polynucleotide:transfection reagent complexes to a cell culture of about 600 liters. In some embodiments, a method described herein comprises transferring 4 separately produced about 20-liter volumes of polynucleotide:transfection reagent complexes to a cell culture of about 800 liters. In some embodiments, a method described herein comprises transferring 5 separately produced about 20-liter volumes of polynucleotide:transfection reagent complexes to a cell culture of about 1,000 liters. In some embodiments, a method described herein comprises transferring 6 separately produced about 20-liter volumes of polynucleotide:transfection reagent complexes to a cell culture of about 1,200 liters. In some embodiments, a method described herein comprises transferring 7 separately produced about 20-liter volumes of polynucleotide:transfection reagent complexes to a cell culture of about 1,400 liters. In some embodiments, a method described herein comprises transferring 8 separately produced about 20-liter volumes of polynucleotide:transfection reagent complexes to a cell culture of about 1,600 liters. In some embodiments, a method described herein comprises transferring 9 separately produced about 20-liter volumes of polynucleotide:transfection reagent complexes to a cell culture of about 1,800 liters. In some embodiments, a method described herein comprises transferring 10 separately produced about 20-liter volumes of polynucleotide:transfection reagent complexes to a cell culture of about 2,000 liters. For reference, non-limiting examples of the volumes of separately produced transfection complex mixtures are provided in Table 1.

In some embodiments, a method described herein comprises transferring 1 about 40-liter volumes, such as about 42 liters of polynucleotide:transfection reagent complexes to a cell culture of about 400 liters. In some embodiments, a method described herein comprises transferring 2 separately produced about 40-liter volumes of polynucleotide:transfection reagent complexes to a cell culture of about 800 liters. In some embodiments, a method described herein comprises transferring 4 separately produced about 40-liter volumes of polynucleotide:transfection reagent complexes to a cell culture of about 1600 liters. For reference, non-limiting examples of the volumes of separately produced transfection complex mixtures are provided in Table 1.

TABLE 1

| No. of | | | | | | | |
|---|---|---|---|---|---|---|---|
| separately | | | | | | | |
| produced | | | | | | | |
| Transfection | | Total | | Cell Culture | | | |
| Complex | | Transfection | | volume prior to | | | |
| mixtures, | | Complex | | Transfection | | Final Bioreactor/ | |
| and volume | | volume | | Complex addition | | Vessel capacity | |
| 1 × 4 | L | 4 | L | 40 | L | 50 | L |
| 4 × 4 | L | 16 | L | 160 | L | 200 | L |
| 2 × 8 | L | 16 | L | 160 | L | 200 | L |
| 1 × 16 | L | 16 | L | 160 | L | 200 | L |
| 2 × 21 | L | 42 | L | 420 | L | 500 | L |
| 1 × 42 | L | 42 | L | 420 | L | 500 | L |
| 4 × 20 | L | 80 | L | 800 | L | 1,000 | L |
| 2 × 40 | L | 80 | L | 800 | L | 1,000 | L |
| 8 × 20 | L | 160 | L | 1,600 | L | 2,000 | L |
| 4 × 40 | L | 160 | L | 1,600 | L | 2,000 | L |
| 2 × 80 | L | 160 | L | 1,600 | L | 2,000 | L |
| 8 × 100 | L | 800 | L | 8,000 | L | 10,000 | L |
| 2 × 400 | L | 800 | L | 8,000 | L | 10,000 | L |
| 8 × 200 | L | 1,600 | L | 16,000 | L | 20,000 | L |
| 2 × 800 | L | 1,600 | L | 16,000 | L | 20,000 | L |

In some embodiments, the rate of transfer is set such that the admixing, incubating and transferring is completed in in less than about 90 minutes, about 60 minutes, about 50 minutes, about 40 minutes, about 35 minutes, about 30 minutes, about 25 minutes, or about 20 minutes. In some embodiments, the admixing, incubating and transferring is completed in less than about 30 minutes. In some embodiments, the one or more polynucleotides contain genes necessary for producing of recombinant AAV particles. In some embodiments, the transfection reagent comprises PEI. In some embodiments, the one or more polynucleotides comprise one or more helper genes, rep genes, cap genes and transgenes (for example genes of interest or the rAAV genome to be packaged). In some embodiments, the culture comprises HEK293 cells, such as HEK293 cells adapted for suspension culture.

In some embodiments, a method described herein comprises transferring 2 separately produced about 30-liter volumes of polynucleotide:transfection reagent complexes to a cell culture of about 600 liters. In some embodiments, a method described herein comprises transferring 3 separately produced about 30-liter volumes of polynucleotide:transfection reagent complexes to a cell culture of about 900 liters. In some embodiments, a method described herein comprises transferring 4 separately produced about 30-liter volumes of polynucleotide:transfection reagent complexes to a cell culture of about 1,200 liters. In some embodiments, a method described herein comprises transferring 5 separately produced about 30-liter volumes of polynucleotide:transfection reagent complexes to a cell culture of about 1,500 liters. In some embodiments, a method described herein comprises transferring 6 separately produced about 30-liter volumes of polynucleotide:transfection reagent complexes to a cell culture of about 1,800 liters. In some embodiments, a method described herein comprises transferring 7 separately produced about 30-liter volumes of polynucleotide:transfection reagent complexes to a cell culture of about 2,100 liters. In some embodiments, a method described herein comprises transferring 8 separately produced about 30-liter volumes of polynucleotide:transfection reagent complexes to a cell culture of about 2,400 liters. In some embodiments, a method described herein comprises transferring 9 separately produced about 30-liter volumes of polynucleotide:transfection reagent complexes to a cell culture of about 2,700 liters. In some embodiments, a method described herein comprises transferring 10 separately produced about 30-liter volumes of polynucleotide:transfection reagent complexes to a cell culture of about 3,000 liters. In some embodiments, the rate of transfer is set such that the admixing, incubating and transferring is completed in in less than about 90 minutes, about 60 minutes, about 50 minutes, about 40 minutes, about 35 minutes, about 30 minutes, about 25 minutes, or about 20 minutes. In some embodiments, the admixing, incubating and transferring is completed in less than about 30 minutes. In some embodiments, the one or more polynucleotides contain the genes necessary for producing of recombinant AAV particles. In some embodiments, the transfection reagent comprises PEI. In some embodiments, the culture comprises HEK293 cells, such as HEK293 cells adapted for suspension culture.

In some embodiments, the admixing, incubating and transferring is completed in less than about 90 minutes, about 60 minutes, about 50 minutes, about 40 minutes, about 35 minutes, about 30 minutes, about 25 minutes, or about 20 minutes. In some embodiments, the admixing, incubating and transferring is completed in less than about 60 minutes. In some embodiments, the admixing, incubating and transferring is completed in less than about 50 minutes. In some embodiments, the admixing, incubating and transferring is completed in less than about 40 minutes. In some embodiments, the admixing, incubating and transferring is completed in less than about 35 minutes. In some embodiments, the admixing, incubating and transferring is completed in less than about 30 minutes. In some embodiments, the admixing, incubating and transferring is completed in less than about 25 minutes. In some embodiments, the admixing, incubating and transferring is completed in less than about 20 minutes.

Transferring of the separately produced polynucleotide:transfection reagent complexes to the cell culture can be performed simultaneously or consecutively. Simultaneous transfer comprises overlapping transfer. In some embodiments, the separately produced polynucleotide:transfection reagent complexes are transferred to the cell culture simultaneously. In some embodiments, the separately produced polynucleotide:transfection reagent complexes are transferred to the cell culture consecutively. In some embodiments, the transferring of a volume of separately produced polynucleotide:transfection reagent complexes starts before completing the preceding transfer of a separately produced volume. In some embodiments, the transferring of a volume of separately produced polynucleotide:transfection reagent complexes starts immediately after completing the preceding transfer of a separately produced volume. In some embodiments, the transferring of a volume of separately produced polynucleotide:transfection reagent complexes starts between about 5 minute and about 60 minutes after completing the preceding transfer of a separately produced volume. In some embodiments, the transferring of a volume of separately produced polynucleotide:transfection reagent complexes starts no more than about 5 minutes after completing the preceding transfer of a separately produced volume. In some embodiments, the transferring of a volume of separately produced polynucleotide:transfection reagent complexes starts no more than about 10 minutes after completing the preceding transfer of a separately produced volume. In some embodiments, the transferring of a volume of separately produced polynucleotide:transfection reagent complexes starts no more than about 15 minutes after completing the preceding transfer of a separately produced volume. In some embodiments, the transferring of a volume of separately produced polynucleotide:transfection reagent complexes starts no more than about 20 minutes after completing the preceding transfer of a separately produced volume. In some embodiments, the transferring of a volume of separately produced polynucleotide:transfection reagent complexes starts no more than about 30 minutes after completing the preceding transfer of a separately produced volume. In some embodiments, the transferring of a volume of separately produced polynucleotide:transfection reagent complexes starts no more than about 45 minutes after completing the preceding transfer of a separately produced volume. In some embodiments, the transferring of a volume of separately produced polynucleotide:transfection reagent complexes starts no more than about 60 minutes after completing the preceding transfer of a separately produced volume. In some embodiments, the one or more polynucleotides contain genes necessary for producing of recombinant AAV particles. In some embodiments, the transfection reagent comprises PEI. In some embodiments, the culture comprises HEK293 cells, such as HEK293 cells adapted for suspension culture.

In some embodiments, the separately produced volumes of polynucleotide:transfection reagent complexes are transferred to the cell culture over a time period that is between about 1 hour and about 12 hours. In some embodiments, the separately produced volumes of polynucleotide:transfection reagent complexes are transferred to the cell culture over a time period that is between about 1 hour and about 8 hours. In some embodiments, the separately produced volumes of polynucleotide:transfection reagent complexes are transferred to the cell culture over a time period that is between about 1 hour and about 6 hours. In some embodiments, the separately produced volumes of polynucleotide:transfection reagent complexes are transferred to the cell culture over a time period that is between about 1 hour and about 5 hours. In some embodiments, the separately produced volumes of polynucleotide:transfection reagent complexes are transferred to the cell culture over a time period that is between about 1 hour and about 4 hours. In some embodiments, the separately produced volumes of polynucleotide:transfection reagent complexes are transferred to the cell culture over a time period that is between about 1 hour and about 3 hours. In some embodiments, the separately produced volumes of polynucleotide:transfection reagent complexes are transferred to the cell culture over a time period that is no longer than about 12 hours. In some embodiments, the separately produced volumes of polynucleotide:transfection reagent complexes are transferred to the cell culture over a time period that is no longer than about 9 hours. In some embodiments, the separately produced volumes of polynucleotide:transfection reagent complexes are transferred to the cell culture over a time period that is no longer than about 8 hours. In some embodiments, the separately produced volumes of polynucleotide:transfection reagent complexes are transferred to the cell culture over a time period that is no longer than about 7 hours. In some embodiments, the separately produced volumes of polynucleotide:transfection reagent complexes are transferred to the cell culture over a time period that is no longer than about 6 hours. In some embodiments, the separately produced volumes of polynucleotide:transfection reagent complexes are transferred to the cell culture over a time period that is no longer than about 5 hours. In some embodiments, the separately produced volumes of polynucleotide:transfection reagent complexes are transferred to the cell culture over a time period that is no longer than about 5 hours. In some embodiments, the separately produced volumes of polynucleotide:transfection reagent complexes are transferred to the cell culture over a time period that is no longer than about 3 hours. In some embodiments, the separately produced volumes of polynucleotide:transfection reagent complexes are transferred to the cell culture over a time period that is no longer than about 2 hours. In some embodiments, the one or more polynucleotides encode the genetic information (contain genes) necessary for producing of recombinant AAV particles. In some embodiments, the one or more polynucleotides comprise one or more helper genes, rep genes, cap genes and transgenes (for example genes of interest or the rAAV genome to be packaged). In some embodiments, the transfection reagent comprises PEI. In some embodiments, the culture comprises HEK293 cells, such as HEK293 cells adapted for suspension culture.

In some embodiments, the cell culture comprises between about 2×10E+6 and about 10E+7 viable cell/ml. In some embodiments, the cell culture comprises between about 3×10E+6 and about 8×10E+6 viable cell/ml. In some embodiments, the cell culture comprises about 3×10E+6 viable cell/ml. In some embodiments, the cell culture comprises about 4×10E+6 viable cell/ml. In some embodiments, the cell culture comprises about 5×10E+6 viable cell/ml. In some embodiments, the cell culture comprises about 6×10E+6 viable cell/ml. In some embodiments, the cell culture comprises about 7×10E+6 viable cell/ml. In some embodiments, the cell culture comprises about 8×10E+6 viable cell/ml. In some embodiments, the culture comprises HEK293 cells, such as HEK293 cells adapted for suspension culture.

In some embodiments, the population of cells comprises a population of mammalian cells or insect cells. In some embodiments, the population of cells comprises a population of mammalian cells. In some embodiments, the population of cells comprises a population of HEK293 cells, HEK derived cells, CHO cells, CHO derived cells, HeLa cells, SF-9 cells, BHK cells, Vero cells, and/or PerC6 cells. In some embodiments, the population of cells comprises a population of HEK293 cells.

In some embodiments, the cell culture is maintained for between about 2 days and about 10 days after transferring the polynucleotide:transfection reagent complexes. In some embodiments, the cell culture is maintained for between about 3 days and about 5 days after transferring the polynucleotide:transfection reagent complexes. In some embodiments, the cell culture is maintained for between about 5 days and about 14 days or more after transferring the polynucleotide:transfection reagent complexes. In some embodiments, the cell culture is maintained for between about 2 days and about 7 days after transferring the polynucleotide:transfection reagent complexes. In some embodiments, the cell culture is maintained for between about 3 days and about 5 days after transferring the polynucleotide:transfection reagent complexes. In some embodiments, the cell culture is maintained for about 2 days, about 3 days, about 4 days, about 5 days, about 6 days, or about 7 days after transferring the polynucleotide:transfection reagent complexes. In some embodiments, the cell culture is maintained for at least about 3 days after transferring the polynucleotide:transfection reagent complexes. In some embodiments, the cell culture is maintained for about 5 days after transferring the polynucleotide:transfection reagent complexes. In some embodiments, the cell culture is maintained for about 6 days after transferring the polynucleotide:transfection reagent complexes. In some embodiments, the cell culture is maintained under conditions that allow production of the rAAV particles for continuous harvest. In some embodiments, the culture comprises HEK293 cells, such as HEK293 cells adapted for suspension culture.

In some embodiments, a method disclosed herein increases production of recombinant viral particles (e.g., rAAV particles) relative to a reference method comprising a single step of admixing, incubating and transferring the same volume of polynucleotide:transfection reagent complexes. In some embodiments, a method disclosed herein increases recombinant virus production by at least about 50%, at least about 75%, or at least about 100%. In some embodiments, a method disclosed herein increases recombinant virus production by at least about two-fold, at least about three-fold, or at least about five-fold. In some embodiments, a method disclosed herein increases rAAV production by at least about two-fold. In some embodiments, the increase in production is determined by comparing the recombinant virus (e.g., rAAV) titer in the production culture. In some embodiments, recombinant virus (e.g., rAAV) titer is measured as genome copy (GC) per milliliter of the production culture. In some embodiments, the recombinant virus is rAAV. In some embodiments, the rAAV particles comprise a capsid protein from an AAV capsid serotype selected from AAV8 and AAV9. In some embodiments, the rAAV particles have an AAV capsid serotype of AAV8. In some embodiments, the rAAV particles have an AAV capsid serotype of AAV9. In some embodiments, the rAAV particles have a capsid serotype selected from the group consisting of AAV.rh8, AAV.rh10, AAV.rh20, AAV.rh39, AAV.Rh74, AAV.RHM4-1, AAV.hu37, AAV.PHB, and AAV.7m8. In some embodiments, the rAAV particles have a capsid protein with high sequence homology to AAV8 or AAV9 such as, AAV.rh10, AAV.rh20, AAV.rh39, AAV.Rh74, AAV.RHM4-1, and AAV.hu37.

In some embodiments, a method disclosed herein increases production of rAAV particles while maintaining or improving the quality attributes of the rAAV particles and compositions comprising thereof. In some embodiments, the quality of rAAV particles and compositions comprising thereof is assessed by determining the concentration of rAAV particles (e.g., GC/ml), the percentage of particles comprising a copy of the rAAV genome; the ratio of particles without a genome, infectivity of the rAAV particles, stability of rAAV particles, concentration of residual host cell proteins, or concentration of residual host cell nucleic acids (e.g., host cell genomic DNA, plasmid encoding rep and cap genes, plasmid encoding helper functions, plasmid encoding rAAV genome). In some embodiments, the quality of rAAV particles produced by a method disclosed herein or compositions comprising thereof is the same as that of rAAV particles or compositions produced by a reference method comprising a single step of admixing, incubating and transferring the same volume of polynucleotide:transfection reagent complexes. In some embodiments, the quality of rAAV particles produced by a method disclosed herein or compositions comprising thereof is better than the quality of rAAV particles or compositions produced by a reference method comprising a single step of admixing, incubating and transferring the same volume of polynucleotide:transfection reagent complexes.

In some embodiments, a method disclosed herein produces between about 1×10e+10 GC/ml and about 1×10e+13 GC/ml rAAV particles. In some embodiments, a method disclosed herein produces between about 1×10e+10 GC/ml and about 1×10e+11 GC/ml rAAV particles. In some embodiments, a method disclosed herein produces between about 5×10e+10 GC/ml and about 1×10e+12 GC/ml rAAV particles. In some embodiments, a method disclosed herein produces between about 5×10e+10 GC/ml and about 1×10e+13 GC/ml rAAV particles. In some embodiments, a method disclosed herein produces between about 1×10e+11 GC/ml and about 1×10e+13 GC/ml rAAV particles. In some embodiments, a method disclosed herein produces between about 5×10e+10 GC/ml and about 5×10e+12 GC/ml rAAV particles. In some embodiments, a method disclosed herein produces between about 1×10e+11 GC/ml and about 5×10e+12 GC/ml rAAV particles. In some embodiments, a method disclosed herein produces more than about 1×10e+11 GC/ml rAAV particles. In some embodiments, a method disclosed herein produces more than about 5×10e+11 GC/ml rAAV particles. In some embodiments, a method disclosed herein produces more than about 1×10e+12 GC/ml rAAV particles. In some embodiments, the rAAV particles comprise a capsid protein from an AAV capsid serotype selected from AAV8 and AAV9. In some embodiments, the rAAV particles have an AAV capsid serotype of AAV8. In some embodiments, the rAAV particles have an AAV capsid serotype of AAV9. In some embodiments, the rAAV particles comprise a capsid protein from an AAV capsid serotype selected from the group consisting of AAV.rh8, AAV.rh10, AAV.rh20, AAV.rh39, AAV.Rh74, AAV.RHM4-1, AAV.hu37, AAV.PHB, and AAV.7m8. In some embodiments, the rAAV particles comprise a capsid protein with high sequence homology to AAV8 or AAV9 such as, AAV.rh10, AAV.rh20, AAV.rh39, AAV.Rh74, AAV.RHM4-1, and AAV.hu37.

In some embodiments, a method disclosed herein produces at least about 5×10e+10 GC/ml rAAV particles. In some embodiments, a method disclosed herein produces at least about 1×10e+11 GC/ml rAAV particles. In some embodiments, a method disclosed herein produces at least about 5×10e+11 GC/ml rAAV particles. In some embodiments, a method disclosed herein produces at least about 1×10e+12 GC/ml rAAV particles. In some embodiments, a method disclosed herein produces at least about 5×10e+12 GC/ml rAAV particles. In some embodiments, a method disclosed herein produces at least about 1×10e+13 GC/ml rAAV particles. In some embodiments, a method disclosed herein produces at least about 5×10e+13 GC/ml rAAV particles. In some embodiments, the rAAV particles comprise a capsid protein from an AAV capsid serotype selected from AAV8 and AAV9. In some embodiments, the rAAV particles have an AAV capsid serotype of AAV8. In some embodiments, the rAAV particles have an AAV capsid serotype of AAV9. In some embodiments, the rAAV particles comprise a capsid protein from an AAV capsid serotype selected from the group consisting of AAV.rh8, AAV.rh10, AAV.rh20, AAV.rh39, AAV.Rh74, AAV.RHM4-1, AAV.hu37, AAV.PHB, and AAV.7m8. In some embodiments, the rAAV particles comprise a capsid protein with high sequence homology to AAV8 or AAV9 such as, AAV.rh10, AAV.rh20, AAV.rh39, AAV.Rh74, AAV.RHM4-1, and AAV.hu37.

Numerous cell culture based systems are known in the art for production of rAAV particles, any of which can be used to practice a method disclosed herein. rAAV production cultures for the production of rAAV virus particles require; (1) suitable host cells, including, for example, human-derived cell lines such as HeLa, A549, or HEK293 cells and their derivatives (HEK293T cells, HEK293F cells), or mammalian cell lines such as Vero, CHO cells or CHO-derived cells; (2) suitable helper virus function, provided by wild type or mutant adenovirus (such as temperature sensitive adenovirus), herpes virus, baculovirus, or a plasmid construct providing helper functions; (3) AAV rep and cap genes and gene products; (4) a transgene (such as a therapeutic transgene) flanked by AAV ITR sequences; and (5) suitable media and media components to support rAAV production.

A skilled artisan is aware of the numerous methods by which AAV rep and cap genes, AAV helper genes (e.g., adenovirus E1a gene, E1b gene, E4 gene, E2a gene, and VA gene), and rAAV genomes (comprising one or more genes of interest flanked by inverted terminal repeats (ITRs)) can be introduced into cells to produce or package rAAV. The phrase “adenovirus helper functions” refers to a number of viral helper genes expressed in a cell (as RNA or protein) such that the AAV grows efficiently in the cell. The skilled artisan understands that helper viruses, including adenovirus and herpes simplex virus (HSV), promote AAV replication and certain genes have been identified that provide the essential functions, e.g. the helper may induce changes to the cellular environment that facilitate such AAV gene expression and replication. In some embodiments of a method disclosed herein, AAV rep and cap genes, helper genes, and rAAV genomes are introduced into cells by transfection of one or more plasmid vectors encoding the AAV rep and cap genes, helper genes, and rAAV genome.

Molecular biology techniques to develop plasmid or viral vectors encoding the AAV rep and cap genes, helper genes, and/or rAAV genome are commonly known in the art. In some embodiments, AAV rep and cap genes are encoded by one plasmid vector. In some embodiments, AAV helper genes (e.g., adenovirus E1a gene, E1b gene, E4 gene, E2a gene, and VA gene) are encoded by one plasmid vector. In some embodiments, the E1a gene or E1b gene is stably expressed by the host cell, and the remaining AAV helper genes are introduced into the cell by transfection by one viral vector. In some embodiments, the E1a gene and E1b gene are stably expressed by the host cell, and the E4 gene, E2a gene, and VA gene are introduced into the cell by transfection by one plasmid vector. In some embodiments, one or more helper genes are stably expressed by the host cell, and one or more helper genes are introduced into the cell by transfection by one plasmid vector. In some embodiments, the helper genes are stably expressed by the host cell. In some embodiments, AAV rep and cap genes are encoded by one viral vector. In some embodiments, AAV helper genes (e.g., adenovirus E1a gene, E1b gene, E4 gene, E2a gene, and VA gene) are encoded by one viral vector. In some embodiments, the E1a gene or E1b gene is stably expressed by the host cell, and the remaining AAV helper genes are introduced into the cell by transfection by one viral vector. In some embodiments, the E1a gene and E1b gene are stably expressed by the host cell, and the E4 gene, E2a gene, and VA gene are introduced into the cell by transfection by one viral vector. In some embodiments, one or more helper genes are stably expressed by the host cell, and one or more helper genes are introduced into the cell by transfection by one viral vector. In some embodiments, the AAV rep and cap genes, the adenovirus helper functions necessary for packaging, and the rAAV genome to be packaged are introduced to the cells by transfection with one or more polynucleotides, e.g., vectors. In some embodiments, a method disclosed herein comprises transfecting the cells with a mixture of three polynucleotides: one encoding the cap and rep genes, one encoding adenovirus helper functions necessary for packaging (e.g., adenovirus E1a gene, E1b gene, E4 gene, E2a gene, and VA gene), and one encoding the rAAV genome to be packaged. In some embodiments, the AAV cap gene is an AAV8 or AAV9 cap gene. In some embodiments, the AAV cap gene is an AAV.rh8, AAV.rh10, AAV.rh20, AAV.rh39, AAV.Rh74, AAV.RHM4-1, AAV.hu37, AAV.PHB, or AAV.7m8 cap gene. In some embodiments, the AAV cap gene encodes a capsid protein with high sequence homology to AAV8 or AAV9 such as, AAV.rh10, AAV.rh20, AAV.rh39, AAV.Rh74, AAV.RHM4-1, and AAV.hu37. In some embodiments, the vector encoding the rAAV genome to be packaged comprises a gene of interest flanked by AAV ITRs. In some embodiments, the AAV ITRs are from AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other AAV serotype.

Any combination of vectors can be used to introduce AAV rep and cap genes, AAV helper genes, and rAAV genome to a cell in which rAAV particles are to be produced or packaged. In some embodiments of a method disclosed herein, a first plasmid vector encoding an rAAV genome comprising a gene of interest flanked by AAV inverted terminal repeats (ITRs), a second vector encoding AAV rep and cap genes, and a third vector encoding helper genes can be used. In some embodiments, a mixture of the three vectors is co-transfected into a cell.

In some embodiments, a combination of transfection and infection is used by using both plasmid vectors as well as viral vectors.

In some embodiments, one or more of rep and cap genes, and AAV helper genes are constitutively expressed by the cells and does not need to be transfected or transduced into the cells. In some embodiments, the cell constitutively expresses rep and/or cap genes. In some embodiments, the cell constitutively expresses one or more AAV helper genes. In some embodiments, the cell constitutively expresses E1a. In some embodiments, the cell comprises a stable transgene encoding the rAAV genome.

In some embodiments, AAV rep, cap, and helper genes (e.g., E1a gene, E1b gene, E4 gene, E2a gene, or VA gene) can be of any AAV serotype. Similarly, AAV ITRs can also be of any AAV serotype. For example, in some embodiments, AAV ITRs are from AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other AAV serotypes (e.g., a hybrid serotype harboring sequences from more than one serotype). In some embodiments, AAV cap gene is from AAV9 or AAV8 cap gene. In some embodiments, an AAV cap gene is from AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other AAV serotypes (e.g., a hybrid serotype harboring sequences from more than one serotype). In some embodiments, AAV rep and cap genes for the production of a rAAV particle is from different serotypes. For example, the rep gene is from AAV2 whereas the cap gene is from AAV9.

Any suitable media known in the art can be used for the production of recombinant virus particles (e.g., rAAV particles) according to a method disclosed herein. These media include, without limitation, media produced by Hyclone Laboratories and JRH including Modified Eagle Medium (MEM), Dulbecco's Modified Eagle Medium (DMEM), and Sf-900 II SFM media as described in U.S. Pat. No. 6,723,551, which is incorporated herein by reference in its entirety. In some embodiments, the medium comprises Dynamis™ Medium, FreeStyle™ 293 Expression Medium, or Expi293™ Expression Medium from Invitrogen/ThermoFisher. In some embodiments, the medium comprises Dynamis™ Medium. In some embodiments, a method disclosed herein uses a cell culture comprising a serum-free medium, an animal-component free medium, or a chemically defined medium. In some embodiments, the medium is an animal-component free medium. In some embodiments, the medium comprises serum. In some embodiments, the medium comprises fetal bovine serum. In some embodiments, the medium is a glutamine-free medium. In some embodiments, the medium comprises glutamine. In some embodiments, the medium is supplemented with one or more of nutrients, salts, buffering agents, and additives (e.g., antifoam agent). In some embodiments, the medium is supplemented with glutamine. In some embodiments, the medium is supplemented with serum. In some embodiments, the medium is supplemented with fetal bovine serum. In some embodiments, the medium is supplemented with poloxamer, e.g., Kolliphor® P 188 Bio. In some embodiments, a medium is a base medium. In some embodiments, the medium is a feed medium.

Recombinant virus (e.g., rAAV) production cultures can routinely be grown under a variety of conditions (over a wide temperature range, for varying lengths of time, and the like) suitable to the particular host cell being utilized. As is known in the art, virus production cultures include suspension-adapted host cells such as HeLa cells, HEK293 cells, HEK293 derived cells (e.g., HEK293T cells, HEK293F cells), Vero cells, CHO cells, CHO-K1 cells, CHO derived cells, EB66 cells, BSC cells, HepG2 cells, LLC-MK cells, CV-1 cells, COS cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, NS-1 cells, MRC-5 cells, WI-38 cells, BHK cells, 3T3 cells, 293 cells, RK cells, Per.C6 cells, chicken embryo cells and SF-9 cells which can be cultured in a variety of ways including, for example, spinner flasks, stirred tank bioreactors, and disposable systems such as the Wave bag system. Numerous suspension cultures are known in the art for production of rAAV particles, including for example, the cultures disclosed in U.S. Pat. Nos. 6,995,006, 9,783,826, and in U.S. Pat. Appl. Pub. No. 20120122155, each of which is incorporated herein by reference in its entirety. In some embodiments, the recombinant virus is recombinant AAV.

Any cell or cell line that is known in the art to produce a recombinant virus particles (e.g., rAAV particles) can be used in any one of the methods disclosed herein. In some embodiments, a method of producing recombinant virus particles (e.g., rAAV particles) or increasing the production of recombinant virus particles (e.g., a rAAV particles) disclosed herein uses HeLa cells, HEK293 cells, HEK293 derived cells (e.g., HEK293T cells, HEK293F cells), Vero cells, CHO cells, CHO-K1 cells, CHO derived cells, EB66 cells, LLC-MK cells, MDCK cells, RAF cells, RK cells, TCMK-1 cells, PK15 cells, BHK cells, BHK-21 cells, NS-1 cells, BHK cells, 293 cells, RK cells, Per.C6 cells, chicken embryo cells or SF-9 cells. In some embodiments, a method disclosed herein uses mammalian cells. In some embodiments, a method disclosed herein uses insect cells, e.g., SF-9 cells. In some embodiments, a method disclosed herein uses cells adapted for growth in suspension culture. In some embodiments, a method disclosed herein uses HEK293 cells adapted for growth in suspension culture. In some embodiments, the recombinant virus particles are recombinant AAV particles.

In some embodiments, a cell culture disclosed herein is a suspension culture. In some embodiments, a large scale suspension cell culture disclosed herein comprises HEK293 cells adapted for growth in suspension culture. In some embodiments, a cell culture disclosed herein comprises a serum-free medium, an animal-component free medium, or a chemically defined medium. In some embodiments, a cell culture disclosed herein comprises a serum-free medium. In some embodiments, suspension-adapted cells are cultured in a shaker flask, a spinner flask, a cellbag, or a bioreactor.

In some embodiments, a cell culture disclosed herein comprises a serum-free medium, an animal-component free medium, or a chemically defined medium. In some embodiments, a cell culture disclosed herein comprises a serum-free medium.

In some embodiments, a cell culture disclosed herein comprises an anti-clumping agent. In some embodiments, a cell culture disclosed herein comprises dextran sulfate. In some embodiments, a cell culture disclosed herein comprises dextran sulfate between about 0.1 mg/L and about 10 mg/L dextran sulfate. Methods for transfecting a host cell in a culture medium comprising dextran sulfate are disclosed in U.S. Provisional Application No. 63/139,992, filed Jan. 21, 2021, titled “Improved production of recombinant polypeptides and viruses,” which is incorporated by reference in its entirety.

In some embodiments, a large scale suspension culture cell culture disclosed herein comprises a high density cell culture. In some embodiments, the culture has a total cell density of between about 1×10E+06 cells/ml and about 30×10E+06 cells/ml. In some embodiments, more than about 50% of the cells are viable cells. In some embodiments, the cells are HeLa cells, HEK293 cells, HEK293 derived cells (e.g., HEK293T cells, HEK293F cells), Vero cells, or SF-9 cells. In further embodiments, the cells are HEK293 cells.

Methods disclosed herein can be used in the production of rAAV particles comprising a capsid protein from any AAV capsid serotype. In some embodiments, the rAAV particles comprise a capsid protein from an AAV capsid serotype selected from AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise a capsid protein that is a derivative, modification, or pseudotype of AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 capsid protein.

In some embodiments, the rAAV particles comprise a capsid protein from an AAV capsid serotype selected from AAV8 and AAV9. In some embodiments, the rAAV particles have an AAV capsid serotype of AAV8. In some embodiments, the rAAV particles have an AAV capsid serotype of AAV9.

In some embodiments, the rAAV particles comprise a capsid protein from an AAV capsid serotype selected from the group consisting of AAV.rh8, AAV.rh10, AAV.rh20, AAV.rh39, AAV.Rh74, AAV.RHM4-1, AAV.hu37, AAV.PHB, and AAV.7m8. In some embodiments, the rAAV particles comprise a capsid protein with high sequence homology to AAV8 or AAV9 such as, AAV.rh10, AAV.rh20, AAV.rh39, AAV.Rh74, AAV.RHM4-1, and AAV.hu37.

In some embodiments, the rAAV particles comprise a capsid protein that is a derivative, modification, or pseudotype of AAV8 or AAV9 capsid protein. In some embodiments, the rAAV particles comprise a capsid protein that has an AAV8 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV8 capsid protein.

In some embodiments, the rAAV particles comprise a capsid protein that is a derivative, modification, or pseudotype of AAV9 capsid protein. In some embodiments, rAAV particles comprise a capsid protein that has an AAV9 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV9 capsid protein.

In some embodiments, the rAAV particles comprise a capsid protein that has at least 80% or more identity, e.g., 85%, 85%, 87%, 88%, 89%, 90%, 91%, 92%, 93%, 94%, 95%, 96%, 97%, 98%, 99%, 99.5%, etc., i.e. up to 100% identity, to the VP1, VP2 and/or VP3 sequence of AAV.rh8, AAV.rh10, AAV.rh20, AAV.rh39, AAV.Rh74, AAV.RHM4-1, AAV.hu37, AAV.PHB, or AAV.7m8 capsid protein. In some embodiments, the rAAV particles comprise a capsid protein that has at least 80% or more identity, e.g., 85%, 85%, 87%, 88%, 89%, 90%, 91%, 92%, 93%, 94%, 95%, 96%, 97%, 98%, 99%, 99.5%, etc., i.e. up to 100% identity, to the VP1, VP2 and/or VP3 sequence of an AAV capsid protein with high sequence homology to AAV8 or AAV9 such as, AAV.rh10, AAV.rh20, AAV.rh39, AAV.Rh74, AAV.RHM4-1, and AAV.hu37.

In additional embodiments, the rAAV particles comprise a mosaic capsid. In additional embodiments, the rAAV particles comprise a pseudotyped rAAV particle. In additional embodiments, the rAAV particles comprise a capsid containing a capsid protein chimera of two or more AAV capsid serotypes.

rAAV Particles

The provided methods are suitable for use in the production of any isolated recombinant AAV particles. As such, the rAAV can be of any serotype, modification, or derivative, known in the art, or any combination thereof (e.g., a population of rAAV particles that comprises two or more serotypes, e.g., comprising two or more of rAAV2, rAAV8, and rAAV9 particles) known in the art. In some embodiments, the rAAV particles are AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof.

In some embodiments, rAAV particles have a capsid protein from an AAV serotype selected from AAV1, AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or a derivative, modification, or pseudotype thereof. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, rAAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In some embodiments, rAAV particles comprise a capsid protein from an AAV capsid serotype selected from AAV1, AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In some embodiments, rAAV particles comprise the capsid of Anc80 or Anc80L65, as described in Zinn et al., 2015, Cell Rep. 12(6): 1056-1068, which is incorporated by reference in its entirety. In certain embodiments, the rAAV particles comprise the capsid with one of the following amino acid insertions: LGETTRP or LALGETTRP, as described in U.S. Pat. Nos. 9,193,956; 9,458,517; and 9,587,282 and US patent application publication no. 2016/0376323, each of which is incorporated herein by reference in its entirety. In some embodiments, rAAV particles comprise the capsid of AAV.7m8, as described in U.S. Pat. Nos. 9,193,956; 9,458,517; and 9,587,282 and US patent application publication no. 2016/0376323, each of which is incorporated herein by reference in its entirety. In some embodiments, rAAV particles comprise any AAV capsid disclosed in U.S. Pat. No. 9,585,971, such as AAVPHP.B. In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis et al., 2016, Gene Therapy 23: 857-862 and Georgiadis et al., 2018, Gene Therapy 25: 450, each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise the capsids of AAVLK03 or AAV3B, as described in Puzzo et al., 2017, Sci. Transl. Med. 29(9): 418, which is incorporated by reference in its entirety. In some embodiments, rAAV particles comprise any AAV capsid disclosed in U.S. Pat. Nos. 8,628,966; 8,927,514; 9,923,120 and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

In some embodiments, rAAV particles comprise an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, rAAV particles have a capsid protein disclosed in Intl. Appl. Publ. No. WO 2003/052051 (see, e.g., SEQ ID NO: 2), WO 2005/033321 (see, e.g., SEQ ID NOs: 123 and 88), WO 03/042397 (see, e.g., SEQ ID NOs: 2, 81, 85, and 97), WO 2006/068888 (see, e.g., SEQ ID NOs: 1 and 3-6), WO 2006/110689, (see, e.g., SEQ ID NOs: 5-38) WO2009/104964 (see, e.g., SEQ ID NOs: 1-5, 7, 9, 20, 22, 24 and 31), WO 2010/127097 (see, e.g., SEQ ID NOs: 5-38), and WO 2015/191508 (see, e.g., SEQ ID NOs: 80-294), and U.S. Appl. Publ. No. 20150023924 (see, e.g., SEQ ID NOs: 1, 5-10), the contents of each of which is herein incorporated by reference in its entirety. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2), WO 2005/033321 (see, e.g., SEQ ID NOs: 123 and 88), WO 03/042397 (see, e.g., SEQ ID NOs: 2, 81, 85, and 97), WO 2006/068888 (see, e.g., SEQ ID NOs: 1 and 3-6), WO 2006/110689 (see, e.g., SEQ ID NOs: 5-38) WO2009/104964 (see, e.g., SEQ ID NOs: 1-5, 7, 9, 20, 22, 24 and 31), WO 2010/127097 (see, e.g., SEQ ID NOs: 5-38), and WO 2015/191508 (see, e.g., SEQ ID NOs: 80-294), and U.S. Appl. Publ. No. 20150023924 (see, e.g., SEQ ID NOs: 1, 5-10).

Nucleic acid sequences of AAV based viral vectors and methods of making recombinant AAV and AAV capsids are taught, for example, in U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; International Patent Application Nos. PCT/US2015/034799; PCT/EP2015/053335; WO 2003/052051, WO 2005/033321, WO 03/042397, WO 2006/068888, WO 2006/110689, WO2009/104964, WO 2010/127097, and WO 2015/191508, and U.S. Appl. Publ. No. 20150023924.

The provided methods are suitable for use in the production of recombinant AAV encoding a transgene. In certain embodiments, the transgene is selected from Tables 2A-2C. In some embodiments, the rAAV genome comprises a vector comprising the following components: (1) AAV inverted terminal repeats that flank an expression cassette; (2) regulatory control elements, such as a) promoter/enhancers, b) a poly A signal, and c) optionally an intron; and (3) nucleic acid sequences coding for a transgene. In other embodiments for expressing an intact or substantially intact monoclonal antibody (mAb), the rAAV genome comprises a vector comprising the following components: (1) AAV inverted terminal repeats that flank an expression cassette; (2) regulatory control elements, such as a) promoter/enhancers, b) a poly A signal, and c) optionally an intron; and (3) nucleic acid sequences coding for the light chain Fab and heavy chain Fab of the antibody, or at least the heavy chain or light chain Fab, and optionally a heavy chain Fc region. In still other embodiments for expressing an intact or substantially intact mAb, the rAAV genome comprises a vector comprising the following components: (1) AAV inverted terminal repeats that flank an expression cassette; (2) regulatory control elements, such as a) promoter/enhancers, b) a poly A signal, and c) optionally an intron; and (3) nucleic acid sequences coding for the heavy chain Fab of an anti-VEGF (e.g., sevacizumab, ranibizumab, bevacizumab, and brolucizumab), anti-EpoR (e.g., LKA-651,), anti-ALK1 (e.g., ascrinvacumab), anti-C5 (e.g., tesidolumab and eculizumab), anti-CD105 (e.g., carotuximab), anti-CC1Q (e.g., ANX-007), anti-TNFα (e.g., adalimumab, infliximab, and golimumab), anti-RGMa (e.g., elezanumab), anti-TTR (e.g., NI-301 and PRX-004), anti-CTGF (e.g., pamrevlumab), anti-IL6R (e.g., satralizumab and sarilumab), anti-TL4R (e.g., dupilumab), anti-IL17A (e.g., ixekizumab and secukinumab), anti-IL-5 (e.g., mepolizumab), anti-IL12/IL23 (e.g., ustekinumab), anti-CD19 (e.g., inebilizumab), anti-ITGF7 mAb (e.g., etrolizumab), anti-SOST mAb (e.g., romosozumab), anti-pKal mAb (e.g., lanadelumab), anti-ITGA4 (e.g., natalizumab), anti-ITGA4B7 (e.g., vedolizumab), anti-BLyS (e.g., belimumab), anti-PD-1 (e.g., nivolumab and pembrolizumab), anti-RANKL (e.g., densomab), anti-PCSK9 (e.g., alirocumab and evolocumab), anti-ANGPTL3 (e.g., evinacumab*), anti-OxPL (e.g., E06), anti-fD (e.g., lampalizumab), or anti-MMP9 (e.g., andecaliximab); optionally an Fc polypeptide of the same isotype as the native form of the therapeutic antibody, such as an IgG isotype amino acid sequence IgG1, IgG2 or IgG4 or modified Fc thereof, and the light chain of an anti-VEGF (e.g., sevacizumab, ranibizumab, bevacizumab, and brolucizumab), anti-EpoR (e.g., LKA-651,), anti-ALK1 (e.g., ascrinvacumab), anti-C5 (e.g., tesidolumab and eculizumab), anti-CD105 or anti-ENG (e.g., carotuximab), anti-CC1Q (e.g., ANX-007), anti-TNFα (e.g., adalimumab, infliximab, and golimumab), anti-RGMa (e.g., elezanumab), anti-TTR (e.g., NI-301 and PRX-004), anti-CTGF (e.g., pamrevlumab), anti-IL6R (e.g., satralizumab and sarilumab), anti-IL4R (e.g., dupilumab), anti-IL17A (e.g., ixekizumab and secukinumab), anti-IL-5 (e.g., mepolizumab), anti-IL12/IL23 (e.g., ustekinumab), anti-CD19 (e.g., inebilizumab), anti-ITGF7 mAb (e.g., etrolizumab), anti-SOST mAb (e.g., romosozumab), anti-pKal mAb (e.g., lanadelumab), anti-ITGA4 (e.g., natalizumab), anti-ITGA4B7 (e.g., vedolizumab), anti-BLyS (e.g., belimumab), anti-PD-1 (e.g., nivolumab and pembrolizumab), anti-RANKL (e.g., densomab), anti-PCSK9 (e.g., alirocumab and evolocumab), anti-ANGPTL3 (e.g., evinacumab), anti-OxPL (e.g., E06), anti-fD (e.g., lampalizumab), or anti-MMP9 (e.g., andecaliximab); wherein the heavy chain (Fab and optionally Fc region) and the light chain are separated by a self-cleaving furin (F)/F2A or furin (F)/F2A or flexible linker, ensuring expression of equal amounts of the heavy and the light chain polypeptides.

TABLE 2A

| Disease | Transgene |
|---|---|
| MPS I | alpha-L-iduronidase (IDUA) |
| MPS II (Hunter Syndrome) | iduronate-2-sulfatase (IDS) |
| ceroid lipofuscinosis (Batten disease) | (CLN1, CLN2, CLN10, CLN13), a soluble |
| | lysosomal protein (CLN5), a protein in the |
| | secretory pathway (CLN11), two cytoplasmic |
| | proteins that also peripherally associate with |
| | membranes (CLN4, CLN14), and many |
| | transmembrane proteins with different |
| | subcellular locations (CLN3, CLN6, CLN7, |
| | CLN8, CLN12) |
| MPS IIIa (Sanfilippo type A | heparan sulfate sulfatase (also called N- |
| Syndrome) | sulfoglucosamine sulfohydrolase (SGSH)) |
| MPS IIIB (Sanfilippo type B | N-acetyl-alpha-D-glucosaminidase |
| Syndrome) | (NAGLU) |
| MPS VI (Maroteaux-Lamy Syndrome) | arylsulfatase B |
| Disease | Transgene |
| Gaucher disease (type I, II and III) | Glucocerebrosidase, GBA1 |
| Parkinson's Disease | Glucocerebrosidase; GBA1 |
| Parkinson's Disease | dopamine decarboxylase |
| Pompe | acid maltase; GAA |
| Metachromatic leukodystrophy | Aryl sulfatase A |
| MPS VII (Sly syndrome) | beta-glucuronidase |
| MPS VIII | glucosamine-6-sulfate sulfatase |
| MPS IX | Hyaluronidase |
| Niemann-Pick disease | Sphingomyelinase |
| Niemann-Pick disease without | a npc1 gene encoding a |
| sphingomyelinase deficiency | cholesterol metabolizing enzyme |
| Tay-Sachs disease | Alpha subunit of beta-hexosaminidase |
| Sandhoff disease | both alpha and beta subunit of beta- |
| | hexosaminidase |
| Fabry Disease | alpha-galactosidase |
| Fucosidosis | Fucosidase (FUCAl gene) |
| Alpha-mannosidosis | alpha-mannosidase |
| Beta-mannosidosis | Beta-mannosidase |
| Wolman disease | cholesterol ester hydrolase |
| Parkinson's disease | Neurturin |
| Parkinson's disease | glial derived growth factor (GDGF) |
| Parkinson's disease | tyrosine hydroxylase |
| Disease | Transgene |
| Parkinson's disease | glutamic acid decarboxylase. |
| Parkinson's disease | fibroblast growth factor-2 (FGF-2) |
| Parkinson's disease | brain derived growth factor (BDGF) |
| No disease listed (Galactosialidosis | neuraminidase deficiency with |
| (Goldberg syndrome)) | betagalactosidase |
| | deficiency |
| Spinal Muscular Atrophy (SMA) | SMN |
| Friedreich's ataxia | Frataxin |
| Amyotrophic lateral sclerosis (ALS) | SOD1 |
| Glycogen Storage Disease la | Glucose-6-phosphatase |
| XLMTM | MTM1 |
| Crigler Najjar | UGT1A1 |
| CPVT | CASQ2 |
| Rett syndrome | MECP2 |
| Achromatopsia | CNGB3, CNGA3, GNAT2, PDE6C |
| Choroidermia | CDM |
| Danon Disease | LAMP2 |
| Cystic Fibrosis | CFTR |
| Duchenne Muscular Dystrophy | Mini-Dystrophin or Micro-Dystrophin Gene |
| Limb Girdle Muscular Dystrophy Type | human-alpha-sarcoglycan |
| 2C\|Gamma-sarcoglycanopathy | |
| Advanced Heart Failure | SERCA2a |
| Rheumatoid Arthritis | TNFR:Fc Fusion Gene |
| Leber Congenital Amaurosis | GAA |
| Limb Girdle Muscular Dystrophy Type | gamma-sarcoglycan |
| 2C\|Gamma-sarcoglycanopathy | |
| Retinitis Pigmentosa | hMERTK |
| Age-Related Macular Degeneration | sFLT01 |
| Becker Muscular Dystrophy and | huFollistatin344 |
| Sporadic Inclusion Body Myositis | |
| Parkinson's Disease | GDNF |
| Disease | Transgene |
| Metachromatic Leukodystrophy | cuARSA |
| (MLD) | |
| Hepatitis C | anti-HCV shRNA |
| Limb Girdle Muscular Dystrophy Type | hSGCA |
| 2D | |
| Human Immunodeficiency Virus | PG9DP |
| Infections; HIV Infections (HIV-1) | |
| Acute Intermittant Porphyria | PBGD |
| Leber's Hereditary Optical Neuropathy | PIND4v2 |
| Alpha-1 Antitrypsin Deficiency | alpha1AT |
| Pompe Disease | hGAA |
| X-linked Retinoschisis | RS1 |
| Choroideremia | hCHM |
| Giant Axonal Neuropathy | JeT-GAN |
| X-linked Retinoschisis | hRS1 |
| Squamous Cell Head and Neck Cancer; | hAQP1 |
| Radiation Induced Xerostomia | |
| Hemophilia B | Factor IX |
| Homozygous FH | hLDLR |
| Dysferlinopathies | dysferlin transgene (e.g. |
| | rAAVrh74.MHCK7.DYSF.DV) |
| Hemophilia B | AAV6 ZFP nuclease |
| MPS I | AAV6 ZFP nuclease |
| Rheumatoid Arthritis | NF-KB.IFN-β |
| Batten/CLN6 | CLN6 |
| Sanfilippo Disease Type A | hSGSH |
| Osteoarthritis | 5IL-1Ra |
| Achromatopsia | CNGA3 |
| Achromatopsia | CNGB3 |
| Ornithine Transcarbamylase (OTC) | OTC |
| Deficiency | |
| Hemophilia A | Factor VIII |
| Mucopolysaccharidosis II | ZFP nuclease |
| Hemophilia A | ZFP nuclease |
| Wet AMD | anti-VEGF |
| X-Linked Retinitis Pigmentosa | RPGR |
| Mucopolysaccharidosis Type VI | hARSB |
| Leber Hereditary Optic Neuropathy | ND4 |
| X-Linked Myotubular Myopathy | MTM1 |
| Crigler-Najjar Syndrome | UGT1A1 |
| Disease | Transgene |
| Achromatopsia | CNGB3 |
| Retinitis Pigmentosa | hPDE6B |
| X-Linked Retinitis Pigmentosa | RPGR |
| Mucopolysaccharidosis Type 3 B | hNAGLU |
| Duchenne Muscular Dystrophy | GALGT2 |
| Arthritis, Rheumatoid; Arthritis, | TNFR:Fc Fusion Gene |
| Psoriatic; Ankylosing Spondylitis | |
| Idiopathic Parkinson's Disease | Neurturin |
| Alzheimer's Disease | NGF |
| Human Immunodeficiency Virus | tgAAC09 |
| Infections; HIV Infections (HIV-1) | |
| Familial Lipoprotein Lipase Deficiency | LPL |
| Idiopathic Parkinson's Disease | Neurturin |
| Alpha-1 Antitrypsin Deficiency | hAAT |
| Leber Congenital Amaurosis (LCA) 2 | hRPE65v2 |
| Batten Disease; Late Infantile | CLN2 |
| Neuronal Lipofuscinosis | |
| Parkinson's Disease | GAD |
| Sanfilippo Disease Type A/ | N-sulfoglucosamine sulfohydrolase (SGSH) |
| Mucopolysaccharidosis Type IIIA | gene |
| Congestive Heart Failure | SERC2a |
| Becker Muscular Dystrophy and | Follistatin (e.g. |
| Sporadic Inclusion Body Myositis | rAAV.CMV.huFollistatin344) |
| Parkinson's Disease | hAADC-2 |
| Choroideremia | REP1 |
| CEA Specific AAV-DC-CTL | CEA |
| Treatment in Stage IV Gastric Cancer | |
| Gastric Cancer | MUC1-peptide-DC-CTL |
| Leber's Hereditary Optical Neuropathy | scAAV2-PIND4v2 |
| Aromatic Amino Acid Decarboxylase | hAADC |
| Deficiency | |
| Hemophilia B | Factor IX |
| Parkinson's Disease | AADC |
| Leber Hereditary Optic Neuropathy | Genetic: GS010\|Drug: Placebo |
| SMA—Spinal Muscular Atrophy\|Gene | SMN |
| Therapy | |
| Hemophilia A | B-Domain Deleted Factor VIII |
| MPS I | IDUA |
| MPS II | IDS |
| Disease | Transgene |
| CLN3-Related Neuronal Ceroid- | CLN3 |
| Lipofuscinosis (Batten) | |
| Limb-Girdle Muscular Dystrophy, | hSGCB |
| Type 2E | |
| Alzheimer Disease | APOE2 |
| Retinitis Pigmentosa | hMERKTK |
| Retinitis Pigmentosa | RLBP1 |
| Wet AMD or diabetic retinopathy | Anti-VEGF antibody or Anti-VEGF trap |
| | (e.g. one or more extracellular domains of |
| | VEGFR-1 and/or VEGFR-2; e.g. aflibercept) |

TABLE 2B

| | | ANTIBODIES | |
|---|---|---|---|
| ANTIGENS | | (TRANSGENE) | INDICATIONS |
| Nervous | Amyloid beta | Solanezumab | Alzheimer's Disease |
| System | (Aβ or Abeta) | GSK933776 | |
| Targets | peptides | | |
| | derived from | | |
| | APP | | |
| | Sortilin | AL-001 | Frontotemporal dementia |
| | | | (FTD) |
| | Tau protein | ABBV-8E12 | Alzheimer's, Progressive |
| | | UCB-0107 | supranuclear palsy, |
| | | NI-105 (BIIB076) | frontotemporal |
| | | | demential, chronic |
| | | | traumatic |
| | | | encephalopathy, Pick's |
| | | | complex, primary age- |
| | | | related taupathy |
| | Semaphorin- | VX15/2503 | Huntington's disease, |
| | 4D (SEMA4D) | | juvenile Huntington's |
| | | | disease |
| | alpha- | Prasinezumab | Parkinson's disease, |
| | synuclein | NI-202 (BIIB054) | synucleinopathies |
| | | MED-1341 | |
| | superoxide | NI-204 | ALS, Alzheimer's |
| | dismutase-1 | | Disease |
| | (SOD-1) | | |
| | CGRP | eptinezumab, | Migraines, Cluster |
| | Receptor | fremanezumab | headaches |
| | | galcanezumab | |
| Ocular Anti- | VEGF | Sevacizumab | diabetic retinopathy |
| Angiogenic | | | (DR), myopic choroidal |
| Targets | | | neovascularization |
| | | | (mCNV), age-related |
| | | | macular degeneration |
| | | | (AMD), macular edema |
| | VEGF | ranibizumab | Wet AMD |
| | | (LUCENTIS ®) | |
| | | bevacizumab | |
| | | (AVASTIN ®) | |
| | | brolucizumab | |
| | erythropoietin | LKA-651 | retinal vein occlusion |
| | receptor | | (RVO), wet AMD, |
| | | | macular edema |
| | Amyloid beta | Solanezumab | Dry AMD |
| | (Aβ or Abeta) | GSK933776 | |
| | peptides | | |
| | derived from | | |
| | APP | | |
| | activin | ascrinvacumab | neovascular age-related |
| | receptor like | | macular degeneration |
| | kinase 1 | | |
| | (ALK1) | | |
| | complement | tesidolumab | dry AMD, uveitis |
| | component 5 | | |
| | (C5) | | |
| | endoglin (END | carotuximab | wet AMD and other |
| | or CD105) | | retinal disorders caused |
| | | | by increased |
| | | | vascularization |
| | complement | ANX-007 | glaucoma |
| | component 1Q | | |
| | (C1Q) | | |
| TNF-alpha | | adalimumab | uveitis |
| | | (HUMIRA ®) | |
| | | infliximab | |
| | | (REMICADE ®) | |
| | | golimumab | |
| Repulsive guidance molecule-A | | elezanumab | multiple sclerosis |
| Transthyretin (TTR) | | NI-301 | amyloidosis |
| | | PRX-004 | |
| Connective tissue growth factor | | pamrevlumab | fibrotic diseases, e.g. |
| (CTGF) | | | diabetic nephropathy, |
| | | | liver fibrosis, idiopathic |
| | | | pulmonary fibrosis |
| Neuromyelitis | interleukin | Satralizumab | NMO, DR, DME, uveitis |
| optica | receptor 6 | sarilumab | |
| (NMO)/Uveitis | (IL6R) | | |
| targets | CD19 | inebilizumab | NMO |

| | | |
|---|---|---|
| Integrin beta 7 | etrolizumab | ulcerative colitis, |
| | | Crohn's disease |
| Sclerostin | romosozumab | Osteoporosis, abnormal |
| | (EVENITY ®) | bone loss or weakness |

TABLE 2C

| | | ANTIBODIES | |
|---|---|---|---|
| ANTIGENS | | (TRANSGENE) | INDICATIONS |
| Nervous | Amyloid beta (Aβ | Aducanumab | Alzheimer's Disease |
| System | or Abeta) | crenezumab | |
| Targets | peptides | gantenerumab | |
| | Tau protein | anti-TAU | Alzheimer's, Progressive |
| | | | supranuclear palsy, |
| | | | frontotemporal |
| | | | demential, chronic |
| | | | traumatic |
| | | | encephalopathy, Pick's |
| | | | complex, primary age- |
| | | | related taupathy |
| | CGRP Receptor | erenumab | Migraine |
| | | (AIMOVIG ™) | |
| Interleukins or | IL-17A | ixekizumab | Plaque psoriasis, |
| interleukin | | (TALTZ ®) | psoriatic arthritis, |
| receptors | | secukinumab | ankylosing sponylitis |
| | | (COSENTYX ®) | |
| | IL-5 | mepolizumab | Asthma |
| | | (NUCALA ®) | |
| | IL-12/IL-23 | ustekinumab | Psoriasis & Crohn's |
| | | (STELARA ®) | disease |
| | IL-4R | dupilumab | Atopic dermatitis |
| Integrin | | vedolizumab | Ulcerative colitis & |
| | | (ENTYVIO ®) | Crohn's disease |
| | | Natalizumab (anti- | Multiple sclerosis & |
| | | integrin alpha 4) | Crohn's disease |
| Cardiovascular | PCSK9 | alirocumab | HeFH & HoFH |
| Targets | | (PRALUENT ®) | |
| | | evolucomab | |
| | | (REPATHA ®) | |
| | ANGPTL3 | evinacumab | HoFH & severe forms of |
| | | | dyslipidema |
| | Proinflammatory/ | E06-scFv | Cardiovascular diseases |
| | proatherogenic | | such as atherosclerosis |
| | phospholipids | | |
| RANKL | | denosumab | Osteoporosis, increasing |
| | | (XGEVA ® and | bone mass in breast and |
| | | PROLIA ®) | prostate cancer patients, |
| | | | & preventing skeletal- |
| | | | related events due to |
| | | | bone metastasis |
| PD-1, or PD-LI or PD-L2 | | nivolumab | Metastatic melanoma, |
| | | (OPDIVO ®) | lymphoma, non-small |
| | | pembrolizumab | cell lung carcinoma |
| | | (KEYTRUDA ®) | |
| BLyS (B-lymphocyte stimulator, | | belimumab | Systemic lupus |
| also known as B-cell activating | | (BENLYSTA ®) | erythromatosis |
| factor (BAFF)) | | | |
| Ocular | Factor D | lampalizumab | Dry AMD |
| Targets | MMP9 | andecaliximab | Dry AMD |
| TNF-alpha | | adalimumab | Rheumatoid arthritis, |
| | | (HUMIRA ®) and | psoriatic arthritis, |
| | | | askylosing spondylitis, |
| | | | Crohn's disease, plaque |
| | | infliximab | psoriasis, ulcerative |
| | | (REMICADE ®) | colitis |
| Plasma | C5, C5a | eculizumab | Paroxysmal nocturnal |
| Protein | | (SOLIRIS ®) | hemoglobinuria, atypical |
| targets | | | hemolytic uremic |
| | | | syndrome, complement- |
| | | | mediated thrombotic |
| | | | microangiopathy |
| | Plasma kallikrein | lanadelumab | Hereditary angioedema |
| | | | (HAE) |

In some embodiments, provided herein are rAAV viral vectors encoding an anti-VEGF Fab. In specific embodiments, provided herein are rAAV8-based viral vectors encoding an anti-VEGF Fab. In more specific embodiments, provided herein are rAAV8-based viral vectors encoding ranibizumab. In some embodiments, provided herein are rAAV viral vectors encoding iduronidase (IDUA). In specific embodiments, provided herein are rAAV9-based viral vectors encoding IDUA. In some embodiments, provided herein are rAAV viral vectors encoding iduronate 2-sulfatase (IDS). In specific embodiments, provided herein are rAAV9-based viral vectors encoding IDS. In some embodiments, provided herein are rAAV viral vectors encoding a low-density lipoprotein receptor (LDLR). In specific embodiments, provided herein are rAAV8-based viral vectors encoding LDLR. In some embodiments, provided herein are rAAV viral vectors encoding tripeptidyl peptidase 1 (TPP1) protein. In specific embodiments, provided herein are rAAV9-based viral vectors encoding TPP1. In some embodiments, provided herein are rAAV viral vectors encoding non-membrane associated splice variant of VEGF receptor 1 (sFlt-1). In some embodiments, provided herein are rAAV viral vectors encoding gamma-sarcoglycan, Rab Escort Protein 1 (REP1/CHM), retinoid isomerohydrolase (RPE65), cyclic nucleotide gated channel alpha 3 (CNGA3), cyclic nucleotide gated channel beta 3 (CNGB3), aromatic L-amino acid decarboxylase (AADC), lysosome-associated membrane protein 2 isoform B (LAMP2B), Factor VIII, Factor IX, retinitis pigmentosa GTPase regulator (RPGR), retinoschisin (RS1), sarcoplasmic reticulum calcium ATPase (SERCA2a), aflibercept, battenin (CLN3), transmembrane ER protein (CLN6), glutamic acid decarboxylase (GAD), Glial cell line-derived neurotrophic factor (GDNF), aquaporin 1 (AQP1), dystrophin, microdystrophin, myotubularin 1 (MTM1), follistatin (FST), glucose-6-phosphatase (G6Pase), apolipoprotein A2 (APOA2), uridine diphosphate glucuronosyl transferase 1A1 (UGT1A1), arylsulfatase B (ARSB), N-acetyl-alpha-glucosaminidase (NAGLU), alpha-glucosidase (GAA), alpha-galactosidase (GLA), beta-galactosidase (GLB1), lipoprotein lipase (LPL), alpha 1-antitrypsin (AAT), phosphodiesterase 6B (PDE6B), ornithine carbamoyltransferase 90TC), survival motor neuron (SMN1), survival motor neuron (SMN2), neurturin (NRTN), Neurotrophin-3 (NT-3/NTF3), porphobilinogen deaminase (PBGD), nerve growth factor (NGF), mitochondrially encoded NADH:ubiquinone oxidoreductase core subunit 4 (MT-ND4), protective protein cathepsin A (PPCA), dysferlin, MER proto-oncogene, tyrosine kinase (MERTK), cystic fibrosis transmembrane conductance regulator (CFTR), or tumor necrosis factor receptor (TNFR)-immunoglobulin (IgG1) Fc fusion.

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In additional embodiments, rAAV particles comprise a capsid containing a capsid protein chimeric of two or more AAV capsid serotypes. In some embodiments, the capsid protein is a chimeric of 2 or more AAV capsid proteins from AAV serotypes selected from AAV1, AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In certain embodiments, a single-stranded AAV (ssAAV) can be used. In certain embodiments, a self-complementary vector, e.g., scAAV, can be used (see, e.g., Wu, 2007, Human Gene Therapy, 18(2):171-82, McCarty et al, 2001, Gene Therapy, Vol. 8, Number 16, Pages 1248-1254; and U.S. Pat. Nos. 6,596,535; 7,125,717; and 7,456,683, each of which is incorporated herein by reference in its entirety).

In some embodiments, the rAAV particles comprise a capsid protein from an AAV capsid serotype selected from AAV8 or AAV9. In some embodiments, the rAAV particles have an AAV capsid serotype of AAV8. In some embodiments, the rAAV particles have an AAV capsid serotype of AAV9.

In some embodiments, the rAAV particles comprise a capsid protein that is a derivative, modification, or pseudotype of AAV8 or AAV9 capsid protein. In some embodiments, the rAAV particles comprise a capsid protein that has an AAV8 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV8 capsid protein.

In some embodiments, the rAAV particles comprise a capsid protein that is a derivative, modification, or pseudotype of AAV9 capsid protein. In some embodiments, the rAAV particles comprise a capsid protein that has an AAV9 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV9 capsid protein.

In additional embodiments, the rAAV particles comprise a mosaic capsid. Mosaic AAV particles are composed of a mixture of viral capsid proteins from different serotypes of AAV. In some embodiments, the rAAV particles comprise a mosaic capsid containing capsid proteins of a serotype selected from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise a mosaic capsid containing capsid proteins of a serotype selected from AAV1, AAV2, AAV5, AAV6, AAV7, AAV8, AAV9, AAV10, AAVrh.8, AAVrh.10, AAVhu.37, AAVrh.20, and AAVrh.74.

In additional embodiments, the rAAV particles comprise a pseudotyped rAAV particle. In some embodiments, the pseudotyped rAAV particle comprises (a) a nucleic acid vector comprising AAV ITRs and (b) a capsid comprised of capsid proteins derived from AAVx (e.g., AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16). In additional embodiments, the rAAV particles comprise a pseudotyped rAAV particle comprised of a capsid protein of an AAV serotype selected from AAV1, AAV2, AAV5, AAV6, AAV7, AAV8, AAV9, AAV10, AAVrh.8, AAVrh.10, AAVhu.37, AAVrh.20, and AAVrh.74. In additional embodiments, the rAAV particles comprise a pseudotyped rAAV particle containing AAV8 capsid protein. In additional embodiments, the rAAV particles comprise a pseudotyped rAAV particle is comprised of AAV9 capsid protein. In some embodiments, the pseudotyped rAAV8 or rAAV9 particles are rAAV2/8 or rAAV2/9 pseudotyped particles. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In additional embodiments, the rAAV particles comprise a capsid containing a capsid protein chimeric of two or more AAV capsid serotypes. In some embodiments, the rAAV particles comprise an AAV capsid protein chimeric of AAV8 capsid protein and one or more AAV capsid proteins from an AAV serotype selected from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise an AAV capsid protein chimeric of AAV8 capsid protein and one or more AAV capsid proteins from an AAV serotype selected from AAV1, AAV2, AAV5, AAV6, AAV7, AAV9, AAV10, rAAVrh10, AAVrh.8, AAVrh.10, AAVhu.37, AAVrh.20, and AAVrh.74. In some embodiments, the rAAV particles comprise an AAV capsid protein chimeric of AAV9 capsid protein the capsid protein of one or more AAV capsid serotypes selected from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise an AAV capsid protein chimeric of AAV9 capsid protein the capsid protein of one or more AAV capsid serotypes selected from AAV1, AAV2, AAV3, AAV4, AAV5, AA6, AAV7, AAV8, AAV9, AAVrh.8, AAVrh.10, AAVhu.37, AAVrh.20, and AAVrh.74.

Methods for Isolating rAAV Particles

In some embodiments, the disclosure provides methods for producing a composition comprising isolated recombinant adeno-associated virus (rAAV) particles, comprising isolating rAAV particles from a feed comprising an impurity (for example, rAAV production culture). In some embodiments, a method for producing a formulation comprising isolated recombinant adeno-associated virus (rAAV) particles disclosed herein comprises (a) isolating rAAV particles from a feed comprising an impurity (for example, rAAV production culture), and (b) formulating the isolated rAAV particles to produce the formulation.

In some embodiments, the disclosure further provides methods for producing a pharmaceutical unit dosage of a formulation comprising isolated recombinant adeno-associated virus (rAAV) particles, comprising isolating rAAV particles from a feed comprising an impurity (for example, rAAV production culture), and formulating the isolated rAAV particles.

Isolated rAAV particles can be isolated using methods known in the art. In some embodiments, methods of isolating rAAV particles comprises downstream processing such as, for example, harvest of a cell culture, clarification of the harvested cell culture (e.g., by centrifugation or depth filtration), tangential flow filtration, affinity chromatography, anion exchange chromatography, cation exchange chromatography, size exclusion chromatography, hydrophobic interaction chromatography, hydroxylapatite chromatography, sterile filtration, or any combination(s) thereof. In some embodiments, downstream processing includes at least 2, at least 3, at least 4, at least 5 or at least 6 of: harvest of a cell culture, clarification of the harvested cell culture (e.g., by centrifugation or depth filtration), tangential flow filtration, affinity chromatography, anion exchange chromatography, cation exchange chromatography, size exclusion chromatography, hydrophobic interaction chromatography, hydroxylapatite chromatography, and sterile filtration. In some embodiments, downstream processing comprises harvest of a cell culture, clarification of the harvested cell culture (e.g., by depth filtration), sterile filtration, tangential flow filtration, affinity chromatography, and anion exchange chromatography. In some embodiments, downstream processing comprises clarification of a harvested cell culture, sterile filtration, tangential flow filtration, affinity chromatography, and anion exchange chromatography. In some embodiments, downstream processing comprises clarification of a harvested cell culture by depth filtration, sterile filtration, tangential flow filtration, affinity chromatography, and anion exchange chromatography. In some embodiments, clarification of the harvested cell culture comprises sterile filtration. In some embodiments, downstream processing does not include centrifugation. In some embodiments, the rAAV particles comprise a capsid protein of the AAV8 serotype. In some embodiments, the rAAV particles comprise a capsid protein of the AAV9 serotype.

In some embodiments, a method of isolating rAAV particles produced according to a method disclosed herein comprises harvest of a cell culture, clarification of the harvested cell culture (e.g., by depth filtration), a first sterile filtration, a first tangential flow filtration, affinity chromatography, anion exchange chromatography (e.g., monolith anion exchange chromatography or AEX chromatography using a quaternary amine ligand), a second tangential flow filtration, and a second sterile filtration. In some embodiments, a method of isolating rAAV particles disclosed herein comprises harvest of a cell culture, clarification of the harvested cell culture (e.g., by depth filtration), a first sterile filtration, affinity chromatography, anion exchange chromatography (e.g., monolith anion exchange chromatography or AEX chromatography using a quaternary amine ligand), a tangential flow filtration, and a second sterile filtration. In some embodiments, a method of isolating rAAV particles produced according to a method disclosed herein comprises clarification of a harvested cell culture, a first sterile filtration, a first tangential flow filtration, affinity chromatography, anion exchange chromatography (e.g., monolith anion exchange chromatography or AEX chromatography using a quaternary amine ligand), a second tangential flow filtration, and a second sterile filtration. In some embodiments, a method of isolating rAAV particles disclosed herein comprises clarification of a harvested cell culture, a first sterile filtration, affinity chromatography, anion exchange chromatography (e.g., monolith anion exchange chromatography or AEX chromatography using a quaternary amine ligand), tangential flow filtration, and a second sterile filtration. In some embodiments, a method of isolating rAAV particles produced according to a method disclosed herein comprises clarification of a harvested cell culture by depth filtration, a first sterile filtration, a first tangential flow filtration, affinity chromatography, anion exchange chromatography (e.g., monolith anion exchange chromatography or AEX chromatography using a quaternary amine ligand), a second tangential flow filtration, and a second sterile filtration. In some embodiments, a method of isolating rAAV particles disclosed herein comprises clarification of a harvested cell culture by depth filtration, a first sterile filtration, affinity chromatography, anion exchange chromatography (e.g., monolith anion exchange chromatography or AEX chromatography using a quaternary amine ligand), tangential flow filtration, and a second sterile filtration. In some embodiments, the method does not include centrifugation. In some embodiments, clarification of the harvested cell culture comprises sterile filtration. In some embodiments, the rAAV particles comprise a capsid protein of the AAV8 serotype. In some embodiments, the rAAV particles comprise a capsid protein of the AAV9 serotype.

Numerous methods are known in the art for production of rAAV particles, including transfection, stable cell line production, and infectious hybrid virus production systems which include adenovirus-AAV hybrids, herpesvirus-AAV hybrids and baculovirus-AAV hybrids. rAAV production cultures for the production of rAAV virus particles all require; (1) suitable host cells, including, for example, human-derived cell lines such as HeLa, A549, or HEK293 cells and their derivatives (HEK293T cells, HEK293F cells), mammalian cell lines such as Vero, or insect-derived cell lines such as SF-9 in the case of baculovirus production systems; (2) suitable helper virus function, provided by wild type or mutant adenovirus (such as temperature sensitive adenovirus), herpes virus, baculovirus, or a plasmid construct providing helper functions; (3) AAV rep and cap genes and gene products; (4) a transgene (such as a therapeutic transgene) flanked by AAV ITR sequences; and (5) suitable media and media components to support rAAV production. Suitable media known in the art may be used for the production of rAAV vectors. These media include, without limitation, media produced by Hyclone Laboratories and JRH including Modified Eagle Medium (MEM), Dulbecco's Modified Eagle Medium (DMEM), and Sf-900 II SFM media as described in U.S. Pat. No. 6,723,551, which is incorporated herein by reference in its entirety.

rAAV production cultures can routinely be grown under a variety of conditions (over a wide temperature range, for varying lengths of time, and the like) suitable to the particular host cell being utilized. As is known in the art, rAAV production cultures include attachment-dependent cultures which can be cultured in suitable attachment-dependent vessels such as, for example, roller bottles, hollow fiber filters, microcarriers, and packed-bed or fluidized-bed bioreactors. rAAV vector production cultures may also include suspension-adapted host cells such as HeLa cells, HEK293 cells, HEK293 derived cells (e.g., HEK293T cells, HEK293F cells), Vero cells, CHO cells, CHO-K1 cells, CHO derived cells, EB66 cells, BSC cells, HepG2 cells, LLC-MK cells, CV-1 cells, COS cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, NS-1 cells, MRC-5 cells, WI-38 cells, BHK cells, 3T3 cells, 293 cells, RK cells, Per.C6 cells, chicken embryo cells or SF-9 cells which can be cultured in a variety of ways including, for example, spinner flasks, stirred tank bioreactors, and disposable systems such as the Wave bag system. In some embodiments, the cells are HEK293 cells. In some embodiments, the cells are HEK293 cells adapted for growth in suspension culture. Numerous suspension cultures are known in the art for production of rAAV particles, including for example, the cultures disclosed in U.S. Pat. Nos. 6,995,006, 9,783,826, and in U.S. Pat. Appl. Pub. No. 20120122155, each of which is incorporated herein by reference in its entirety.

In some embodiments, the rAAV production culture comprises a high density cell culture. In some embodiments, the culture has a total cell density of between about 1×10E+06 cells/ml and about 30×10E+06 cells/ml. In some embodiments, more than about 50% of the cells are viable cells. In some embodiments, the cells are HeLa cells, HEK293 cells, HEK293 derived cells (e.g., HEK293T cells, HEK293F cells), Vero cells, or SF-9 cells. In further embodiments, the cells are HEK293 cells. In further embodiments, the cells are HEK293 cells adapted for growth in suspension culture.

In additional embodiments of the provided method the rAAV production culture comprises a suspension culture comprising rAAV particles. Numerous suspension cultures are known in the art for production of rAAV particles, including for example, the cultures disclosed in U.S. Pat. Nos. 6,995,006, 9,783,826, and in U.S. Pat. Appl. Pub. No. 20120122155, each of which is incorporated herein by reference in its entirety. In some embodiments, the suspension culture comprises a culture of mammalian cells or insect cells. In some embodiments, the suspension culture comprises a culture of HeLa cells, HEK293 cells, HEK293 derived cells (e.g., HEK293T cells, HEK293F cells), Vero cells, CHO cells, CHO-K1 cells, CHO derived cells, EB66 cells, BSC cells, HepG2 cells, LLC-MK cells, CV-1 cells, COS cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, NS-1 cells, MRC-5 cells, WI-38 cells, BHK cells, 3T3 cells, 293 cells, RK cells, Per.C6 cells, chicken embryo cells or SF-9 cells. In some embodiments, the suspension culture comprises a culture of HEK293 cells.

In some embodiments, methods for the production of rAAV particles encompasses providing a cell culture comprising a cell capable of producing rAAV; adding to the cell culture a histone deacetylase (HDAC) inhibitor to a final concentration between about 0.1 mM and about 20 mM; and maintaining the cell culture under conditions that allows production of the rAAV particles. In some embodiments, the HDAC inhibitor comprises a short-chain fatty acid or salt thereof. In some embodiments, the HDAC inhibitor comprises butyrate (e.g., sodium butyrate), valproate (e.g., sodium valproate), propionate (e.g., sodium propionate), or a combination thereof.

In some embodiments, rAAV particles are produced as disclosed in WO 2020/033842, which is incorporated herein by reference in its entirety.

Recombinant AAV particles can be harvested from rAAV production cultures by harvest of the production culture comprising host cells or by harvest of the spent media from the production culture, provided the cells are cultured under conditions known in the art to cause release of rAAV particles into the media from intact host cells. Recombinant AAV particles can also be harvested from rAAV production cultures by lysis of the host cells of the production culture. Suitable methods of lysing cells are also known in the art and include for example multiple freeze/thaw cycles, sonication, microfluidization, and treatment with chemicals, such as detergents and/or proteases.

At harvest, rAAV production cultures can contain one or more of the following: (1) host cell proteins; (2) host cell DNA; (3) plasmid DNA; (4) helper virus; (5) helper virus proteins; (6) helper virus DNA; and (7) media components including, for example, serum proteins, amino acids, transferrins and other low molecular weight proteins. rAAV production cultures can further contain product-related impurities, for example, inactive vector forms, empty viral capsids, aggregated viral particles or capsids, mis-folded viral capsids, degraded viral particle.

In some embodiments, the rAAV production culture harvest is clarified to remove host cell debris. In some embodiments, the production culture harvest is clarified by filtration through a series of depth filters. Clarification can also be achieved by a variety of other standard techniques known in the art, such as, centrifugation or filtration through any cellulose acetate filter of 0.2 mm or greater pore size known in the art. In some embodiments, clarification of the harvested cell culture comprises sterile filtration. In some embodiments, the production culture harvest is clarified by centrifugation. In some embodiments, clarification of the production culture harvest does not included centrifugation.

In some embodiments, harvested cell culture is clarified using filtration. In some embodiments, clarification of the harvested cell culture comprises depth filtration. In some embodiments, clarification of the harvested cell culture further comprises depth filtration and sterile filtration. In some embodiments, harvested cell culture is clarified using a filter train comprising one or more different filtration media. In some embodiments, the filter train comprises a depth filtration media. In some embodiments, the filter train comprises one or more depth filtration media. In some embodiments, the filter train comprises two depth filtration media. In some embodiments, the filter train comprises a sterile filtration media. In some embodiments, the filter train comprises 2 depth filtration media and a sterile filtration media. In some embodiments, the depth filter media is a porous depth filter. In some embodiments, the filter train comprises Clarisolve® 20MS, Millistak+® C0HC, and a sterilizing grade filter media. In some embodiments, the filter train comprises Clarisolve® 20MS, Millistak+® C0HC, and Sartopore® 2 XLG 0.2 μm. In some embodiments, the harvested cell culture is pretreated before contacting it with the depth filter. In some embodiments, the pretreating comprises adding a salt to the harvested cell culture. In some embodiments, the pretreating comprises adding a chemical flocculent to the harvested cell culture. In some embodiments, the harvested cell culture is not pre-treated before contacting it with the depth filter.

In some embodiments, the production culture harvest is clarified by filtration are disclosed in WO 2019/212921, which is incorporated herein by reference in its entirety.

In some embodiments, the rAAV production culture harvest is treated with a nuclease (e.g., Benzonase®) or endonuclease (e.g., endonuclease from Serratia marcescens) to digest high molecular weight DNA present in the production culture. The nuclease or endonuclease digestion can routinely be performed under standard conditions known in the art. For example, nuclease digestion is performed at a final concentration of 1-2.5 units/ml of Benzonase® at a temperature ranging from ambient to 37° C. for a period of 30 minutes to several hours.

Sterile filtration encompasses filtration using a sterilizing grade filter media. In some embodiments, the sterilizing grade filter media is a 0.2 or 0.22 μm pore filter. In some embodiments, the sterilizing grade filter media comprises polyethersulfone (PES). In some embodiments, the sterilizing grade filter media comprises polyvinylidene fluoride (PVDF). In some embodiments, the sterilizing grade filter media has a hydrophilic heterogeneous double layer design. In some embodiments, the sterilizing grade filter media has a hydrophilic heterogeneous double layer design of a 0.8 μm pre-filter and 0.2 μm final filter membrane. In some embodiments, the sterilizing grade filter media has a hydrophilic heterogeneous double layer design of a 1.2 μm pre-filter and 0.2 μm final filter membrane. In some embodiments, the sterilizing grade filter media is a 0.2 or 0.22 μm pore filter. In further embodiments, the sterilizing grade filter media is a 0.2 μm pore filter. In some embodiments, the sterilizing grade filter media is a Sartopore® 2 XLG 0.2 μm, Durapore™ PVDF Membranes 0.45 μm, or Sartoguard® PES 1.2 m+0.2 m nominal pore size combination. In some embodiments, the sterilizing grade filter media is a Sartopore® 2 XLG 0.2 μm.

In some embodiments, the clarified feed is concentrated via tangential flow filtration (“TFF”) before being applied to a chromatographic medium, for example, affinity chromatography medium. Large scale concentration of viruses using TFF ultrafiltration has been described by Paul et al., Human Gene Therapy 4:609-615 (1993). TFF concentration of the clarified feed enables a technically manageable volume of clarified feed to be subjected to chromatography and allows for more reasonable sizing of columns without the need for lengthy recirculation times. In some embodiments, the clarified feed is concentrated between at least two-fold and at least ten-fold. In some embodiments, the clarified feed is concentrated between at least ten-fold and at least twenty-fold. In some embodiments, the clarified feed is concentrated between at least twenty-fold and at least fifty-fold. In some embodiments, the clarified feed is concentrated about twenty-fold. One of ordinary skill in the art will also recognize that TFF can also be used to remove small molecule impurities (e.g., cell culture contaminants comprising media components, serum albumin, or other serum proteins) form the clarified feed via diafiltration. In some embodiments, the clarified feed is subjected to diafiltration to remove small molecule impurities. In some embodiments, the diafiltration comprises the use of between about 3 and about 10 diafiltration volume of buffer. In some embodiments, the diafiltration comprises the use of about 5 diafiltration volume of buffer. One of ordinary skill in the art will also recognize that TFF can also be used at any step in the purification process where it is desirable to exchange buffers before performing the next step in the purification process. In some embodiments, the methods for isolating rAAV from the clarified feed disclosed herein comprise the use of TFF to exchange buffers.

Affinity chromatography can be used to isolate rAAV particles from a composition. In some embodiments, affinity chromatography is used to isolate rAAV particles from the clarified feed. In some embodiments, affinity chromatography is used to isolate rAAV particles from the clarified feed that has been subjected to tangential flow filtration. Suitable affinity chromatography media are known in the art and include without limitation, AVB Sepharose™, POROS™ CaptureSelect™ AAVX affinity resin, POROS™ CaptureSelect™ AAV9 affinity resin, and POROS™ CaptureSelect™ AAV8 affinity resin. In some embodiments, the affinity chromatography media is POROS™ CaptureSelect™ AAV9 affinity resin. In some embodiments, the affinity chromatography media is POROS™ CaptureSelect™ AAV8 affinity resin. In some embodiments, the affinity chromatography media is POROS™ CaptureSelect™ AAVX affinity resin.

Anion exchange chromatography can be used to isolate rAAV particles from a composition. In some embodiments, anion exchange chromatography is used after affinity chromatography as a final concentration and polish step. Suitable anion exchange chromatography media are known in the art and include without limitation, UNOsphere™ Q (Biorad, Hercules, Calif), and N-charged amino or imino resins such as e.g., POROS™ 50 PI, or any DEAE, TMAE, tertiary or quaternary amine, or PEI-based resins known in the art (U.S. Pat. No. 6,989,264; Brument et al., Mol. Therapy 6(5):678-686 (2002); Gao et al., Hum. Gene Therapy 11:2079-2091 (2000)). In some embodiments, the anion exchange chromatography media comprises a quaternary amine. In some embodiments, the anion exchange media is a monolith anion exchange chromatography resin. In some embodiments, the monolith anion exchange chromatography media comprises glycidylmethacrylate-ethylenedimethacrylate or styrene-divinylbenzene polymers. In some embodiments, the monolith anion exchange chromatography media is selected from the group consisting of CIMmultus™ QA-1 Advanced Composite Column (Quaternary amine), CIMmultus™ DEAE-1 Advanced Composite Column (Diethylamino), CIM® QA Disk (Quaternary amine), CIM® DEAE, and CIM® EDA Disk (Ethylene diamino). In some embodiments, the monolith anion exchange chromatography media is CIMmultus™ QA-1 Advanced Composite Column (Quaternary amine). In some embodiments, the monolith anion exchange chromatography media is CIM® QA Disk (Quaternary amine). In some embodiments, the anion exchange chromatography media is CIM QA (BIA Separations, Slovenia). In some embodiments, the anion exchange chromatography media is BIA CIM® QA-80 (Column volume is 80 mL). One of ordinary skill in the art can appreciate that wash buffers of suitable ionic strength can be identified such that the rAAV remains bound to the resin while impurities, including without limitation impurities which may be introduced by upstream purification steps are stripped away.

In some embodiments, anion exchange chromatography is performed according to a method disclosed in WO 2019/241535, which is incorporated herein by reference in its entirety.

In some embodiments, a method of isolating rAAV particles comprises determining the vector genome titer, capsid titer, and/or the ratio of full to empty capsids in a composition comprising the isolated rAAV particles. In some embodiments, the vector genome titer is determined by quantitative PCR (qPCR) or digital PCR (dPCR) or droplet digital PCR (ddPCR). In some embodiments, the capsid titer is determined by serotype-specific ELISA. In some embodiments, the ratio of full to empty capsids is determined by Analytical Ultracentrifugation (AUC) or Transmission Electron Microscopy (TEM).

In some embodiments, the vector genome titer, capsid titer, and/or the ratio of full to empty capsids is determined by spectrophotometry, for example, by measuring the absorbance of the composition at 260 nm; and measuring the absorbance of the composition at 280 nm. In some embodiments, the rAAV particles are not denatured prior to measuring the absorbance of the composition. In some embodiments, the rAAV particles are denatured prior to measuring the absorbance of the composition. In some embodiments, the absorbance of the composition at 260 nm and 280 nm is determined using a spectrophotometer. In some embodiments, the absorbance of the composition at 260 nm and 280 nm is determined using a IPLC. In some embodiments, the absorbance is peak absorbance. Several methods for measuring the absorbance of a composition at 260 nm and 280 nm are known in the art. Methods of determining vector genome titer and capsid titer of a composition comprising the isolated recombinant rAAV particles are disclosed in WO 2019/212922, which is incorporated herein by reference in its entirety.

In additional embodiments the disclosure provides compositions comprising isolated rAAV particles produced according to a method disclosed herein. In some embodiment, the composition is a pharmaceutical composition comprising a pharmaceutically acceptable carrier.

As used herein the term “pharmaceutically acceptable means a biologically acceptable formulation, gaseous, liquid or solid, or mixture thereof, which is suitable for one or more routes of administration, in vivo delivery or contact. A “pharmaceutically acceptable” composition is a material that is not biologically or otherwise undesirable, e.g., the material may be administered to a subject without causing substantial undesirable biological effects. Thus, such a pharmaceutical composition may be used, for example in administering rAAV isolated according to the disclosed methods to a subject. Such compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions. Pharmaceutical compositions can be formulated to be compatible with a particular route of administration or delivery, as set forth herein or known to one of skill in the art. Thus, pharmaceutical compositions include carriers, diluents, or excipients suitable for administration by various routes. Pharmaceutical compositions and delivery systems appropriate for rAAV particles and methods and uses of the invention are known in the art (see, e.g., Remington: The Science and Practice of Pharmacy (2003) 20th ed., Mack Publishing Co., Easton, Pa.; Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11th ed., Lippincott Williams & Wilkins, Baltimore, Md.; and Poznansky et al., Drug Delivery Systems (1980), R. L. Juliano, ed., Oxford, N.Y., pp. 253-315).

In some embodiments, the composition is a pharmaceutical unit dose. A “unit dose” refers to a physically discrete unit suited as a unitary dosage for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dose forms may be within, for example, ampules and vials, which may include a liquid composition, or a composition in a freeze-dried or lyophilized state; a sterile liquid carrier, for example, can be added prior to administration or delivery in vivo. Individual unit dose forms can be included in multi-dose kits or containers. Recombinant vector (e.g., AAV) sequences, plasmids, vector genomes, and recombinant virus particles, and pharmaceutical compositions thereof can be packaged in single or multiple unit dose form for ease of administration and uniformity of dosage. In some embodiments, the composition comprises rAAV particles comprising an AAV capsid protein from an AAV capsid serotype selected from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the AAV capsid serotype is AAV8. In some embodiments, the AAV capsid serotype is AAV9.

### EXAMPLES

### Example 1—AAV Productivity of a Single Dose Transient Transfection-Based Process in a 500 L Bioreactor was 48-60% of the Productivity Obtained Using 50 L and 200 L Bioreactors

Production of adeno-associated virus (AAV) from cell culture for the gene therapy field has grown from simple adherent flasks to complicated adherent systems to suspension-based stirred-tank bioreactor processes. Many AAV gene therapy production systems exist including transient transfection of mammalian cells, such as suspension adapted HEK293 cells. However, most of the commonly used equipment for modern suspension mammalian biotechnology processes have not been specifically designed for large-scale transient production of AAV.

The challenge of producing an AAV in a suspension mammalian cell transfection process is expanded when comparing bioreactor scale and design. Thus, there is a need to compare current bioreactor systems for production of AAV. The impact of bioreactor configuration and mixing on transfection and AAV production were compared. Bioreactors from 50 L to 500 L were optimized for scalability and process performance. The effect of bioreactor settings and typical scale-up factors on cell growth, viability, and harvest titers were compared for multiple types of bioreactors at the varying scales.

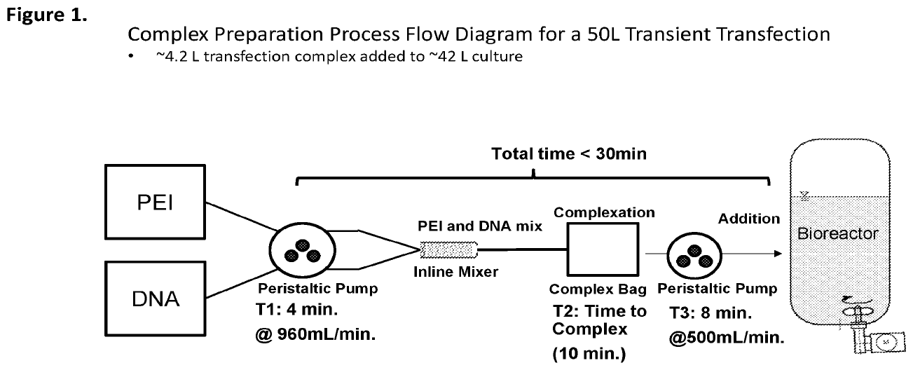

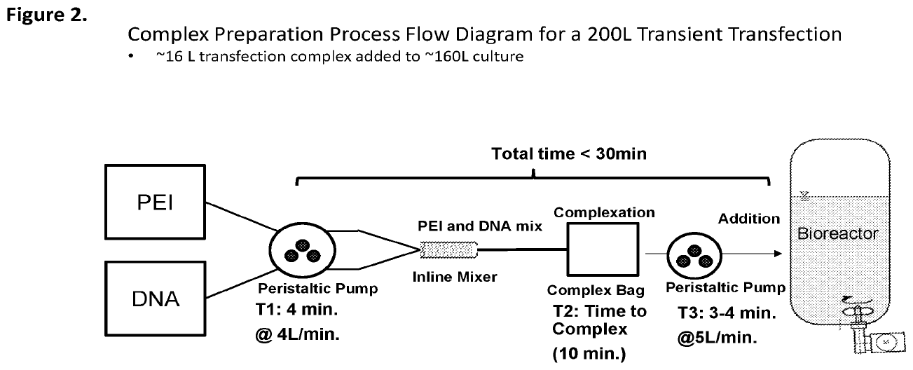

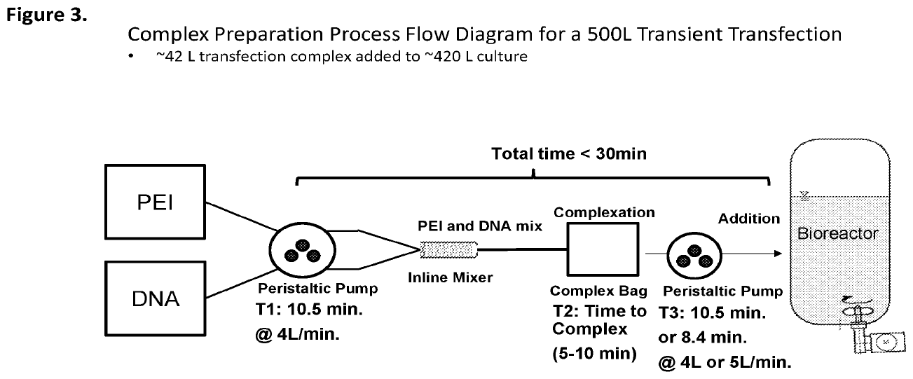

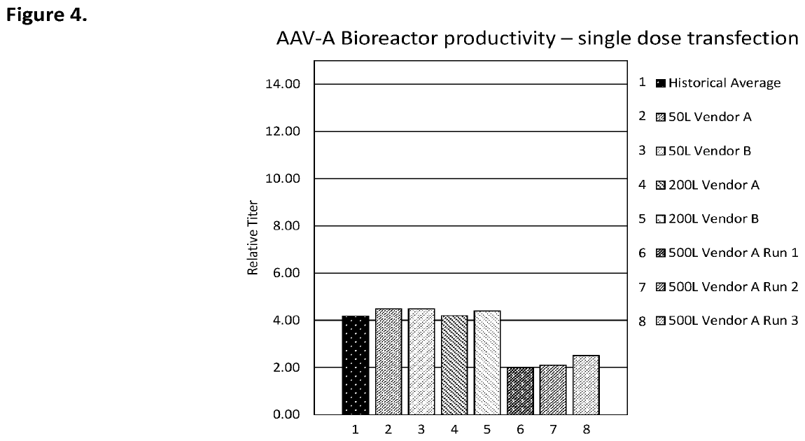

AAV-A vectors (comprising AAV9 capsid) were produced via transient transfection of suspension adapted HEK293 cells substantially as described herein. Briefly, suspension adapted HEK293 cells were thawed and expanded. Bioreactors were seeded with suspension adapted HEK293 cells at a density of about 1.0-1.2×10⁶ viable cells/mL. At 72 hrs. ECD (Elapsed Culture Duration), the cells were transfected with a mixture of polyethylenimine (PEI) and 3 plasmids encoding adeno-virus helper functions, AAV-A transgene and AAV9 Cap/Rep. Transfection was performed using a 1:1.75 DNA:PEI ratio. Transfection complex preparation process flow diagrams for 20 L, 200 L and 500 L bioreactors are shown in FIGS. 1-3. DNA and PEI were mixed using an inline mixer. The volume of the transfection complexes added was approximately 10% of the volume of the cell culture. The supernatant of the cultures was harvested at an ECD of 144-192 hours, e.g., 3-5 days post-transfection. AAV-A yields obtained are shown in FIG. 4. Relative titer was determined as is routine in the art.

The first generation process up to the 500 L scale across multiple vendors of bioreactors (vendor A or B) indicated that cell growth parameters up to the start of production (e.g. up to the transient transfection step) were linear (data not shown). This indicated that P/V and Tip Speed were the optimal scaling parameters for cell growth. During the production stage, cell growth and viability followed similar trends for the 50 L and 200 L bioreactors. Harvest VCD and viabilities for the 500 L were actually higher than the 50 L and 200 L, which points to a lower transfection efficiency. Considering the similar cell growth and viability across the several bioreactors tested in the hands of two different vendors, it was surprising that the 500 L bioreactor productivity of AAV-A was only 48-60% based on an average of four previous production runs, while the 50 L and 200 L bioreactor productivity was in line with average. FIG. 4.

### Example 2—Complexing Time Influences AAV Productivity

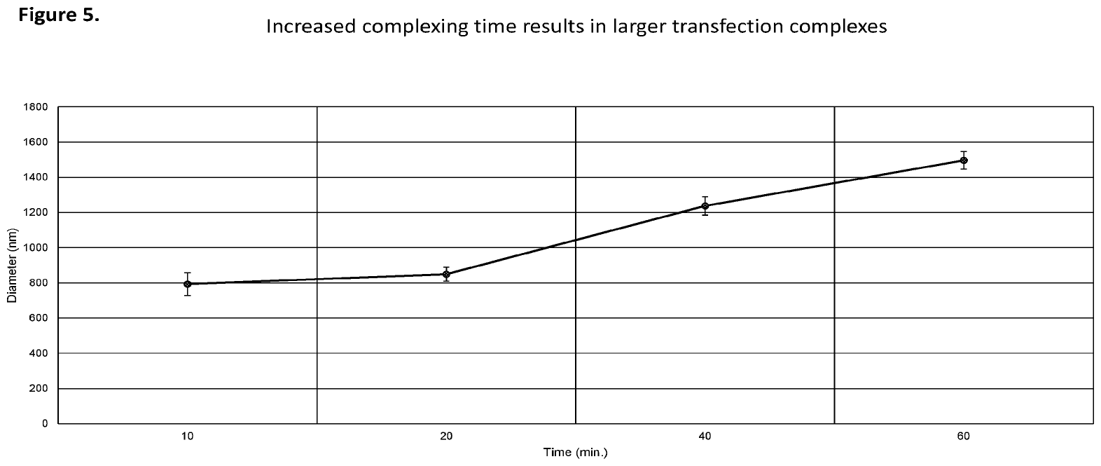

It has been reported that DNA:PEI complexing time influences the size of complexes formed, which in term may influence transfection efficiency. As shown in FIG. 5, increased complexing time resulted in the formation of larger DNA:PEI complexes. Complex size was measured using dynamic light scattering using known methods.

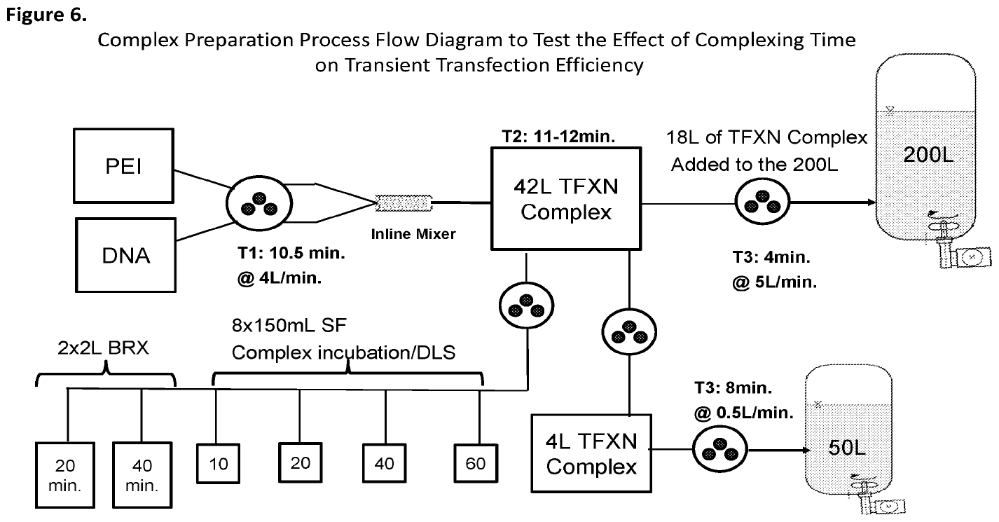

The effect of increased complexing time on AAV-A productivity was tested using the experiment outline in FIG. 6. Briefly, a 42 L transfection complex of PEI and (3) plasmids encoding adeno-virus helper functions, AAV-A transgene, AAV9 Cap/Rep was prepared by mixing the DNA and PEI together via an inline mixer. Once mixed, the transfection complexed was allowed to incubate. Fixed volumes of transfection complex were then removed at pre-determined time points (10, 20, 40, 60 min. after the start of the complex incubation) and used to transfect bioreactors and shake flasks.

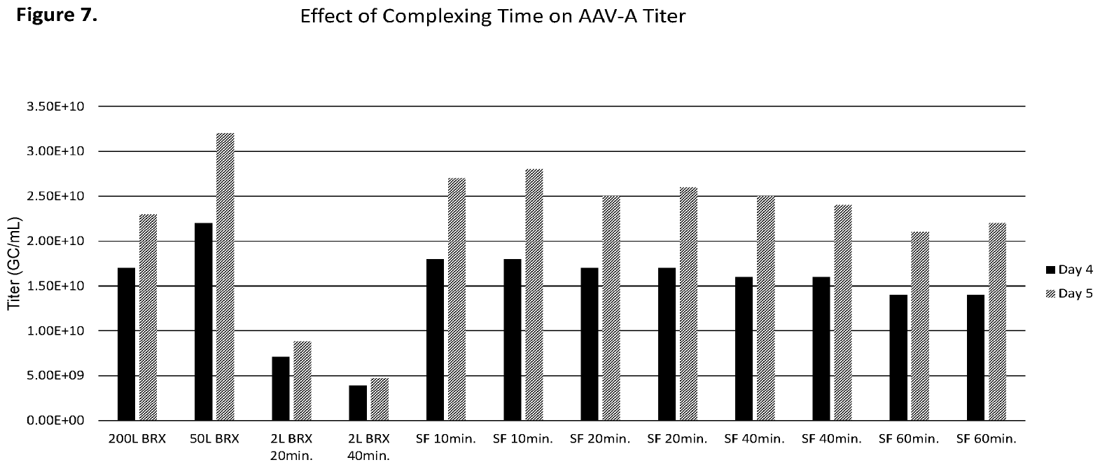

As shown in FIG. 7, increased complexing time resulted in lower AAV productivity. The results of this experiment show a steady decrease in Day 4 and Day 5 post-transfection GC titer with increased transfection complex time.

### Example 3—Single Dose and Split Transient Transfection-Based Processes had Comparable AAV Productivity

Without being bound by a particular theory, a possible explanation for the lower AAV-A productivity observed in Example 1 using a 500 L bioreactor was due to constraints around mixing, complexing, and adding the required volume of transfection complex (42 L for a 500 L bioreactor) during the narrow (30 minute) operating window. As shown in Example 2, the size of DNA:PEI complexes increases with time, and the use of DNA:PEI complexes above an optimal size results in lower AAV-A productivity. It was hypothesized that the combination of a narrow operating window of ˜30 minutes and large volume transfection complex (42 L) led to a low yield (50-60% of expected) due to the fact that the complex incubation and pumping times were potentially unsatisfactory for this large volume of transfection complex.

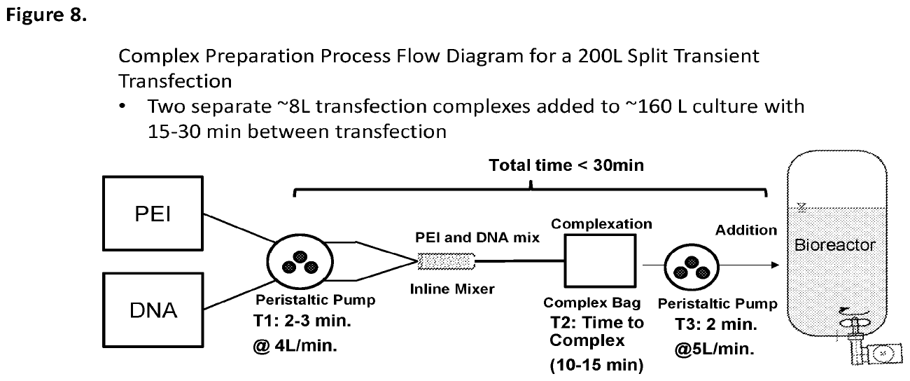

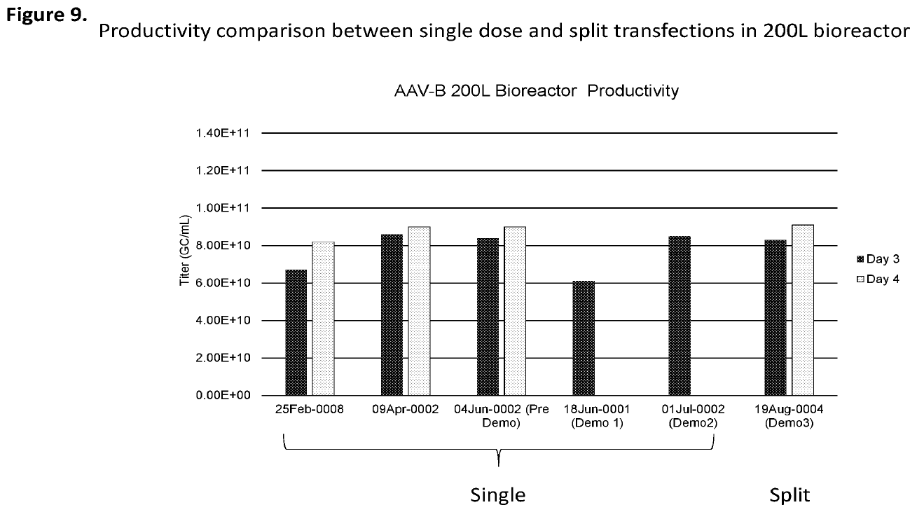

A split transient transfection-based process was tested to determine whether splitting up the transfection process into two or more steps of mixing, complexing, and adding lower volumes of transfection complex to the cell culture instead of a single large volume can be used to eliminate constraints around the transfection complex volume and narrow operating window. A 200 L bioreactor process was used to determine AAV-B (comprising AAV8 capsid) productivity of single dose and split transient transfection-based processes. Single dose transient transfection-based process was performed substantially as described in Example 1. Transfection complex preparation process flow diagrams for the split transfection based process is shown in FIG. 8. The total volume of the transfection complexes added was substantially identical (˜16 L) for the single dose and split transfection processes. While the single dose process involved mixing, complexing and adding the entire volume in one step, the split transfection process used two separate steps of mixing, complexing and adding an ˜8 L volume of transfection complexes, wherein the second adding step was started 15-30 min after completing the first adding step. The supernatant of the cultures was harvested at an ECD of 168 hours ECD, i.e., 4 days post-transfection. AAV-B yields obtained are shown in FIG. 9. The Split Transient Transfection process yielded similar titer to the runs using a single dose of transfection complex. This was an important finding as it addresses the narrow operating window (30 minutes) for preparation and addition of transfection complex volumes for large bioreactors, e.g., 200 L or larger. Splitting the transfection complex into smaller doses allows the use of more robust complex incubation time and pump flow rates within the 30 minute operating window for each dose.

### Example 4—Split Transfection Complex Doses can be Added Several Hours Apart without Significant Loss in AAV Productivity

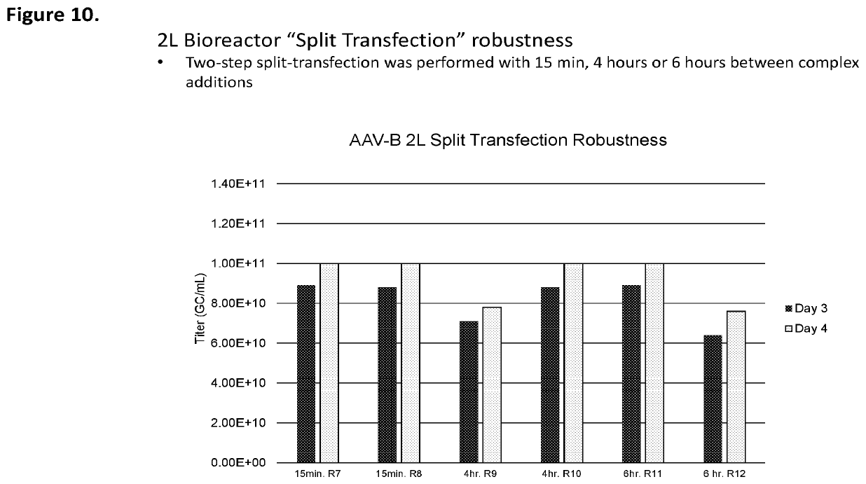

A 2 L bench scale experiment using AAV-B was performed to test the robustness of the Split Transient Transfection process. The purpose was to see how far apart the additions of the split transection complex could be and still get equivalent GC titer. The time interval between split additions of transfection complex varied from 15 minutes to 6 hours. Cell growth and viability trends were similar for the conditions tested (data not shown). Glucose trends were also similar between conditions (data not shown). L-Glutamine and NH4+ trends were similar for all conditions tested up until an ECD of 144 hours. After this point the bioreactors with 15 minutes between the split additions of transfection complex consumed more L-Glutamine and produced more NH4+. This difference had no negative impact on production as these bioreactors had equivalent and in some cases higher GC titer than bioreactors with 4 hours and 6 hours between split additions of the transfection complex. FIG. 10. Although there was slightly more variability in GC titer, the split additions of transfection complex can be added to the bioreactor up to 6 hours apart and still produce titers that are within 80-90% of the expected range.

### Example 5—AAV-B Productivity of Split Transient Transfection-Based Process in a 500 L Bioreactor is Similar to Productivity Obtained Using 50 L and 200 L Bioreactors

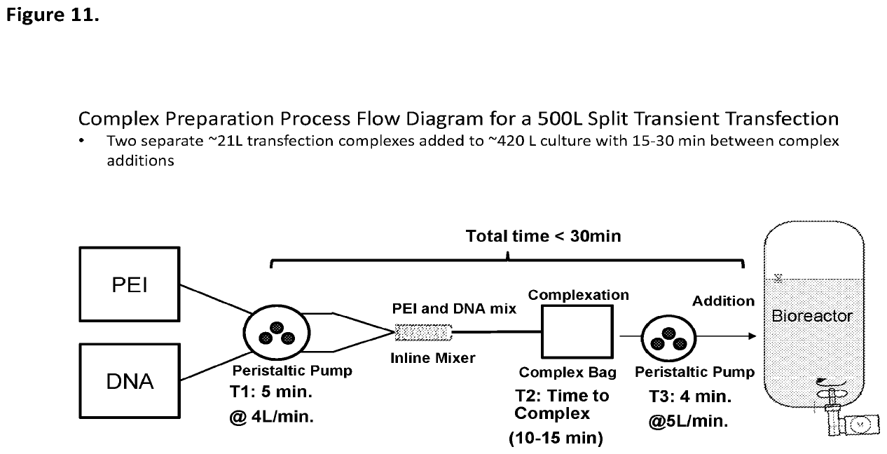

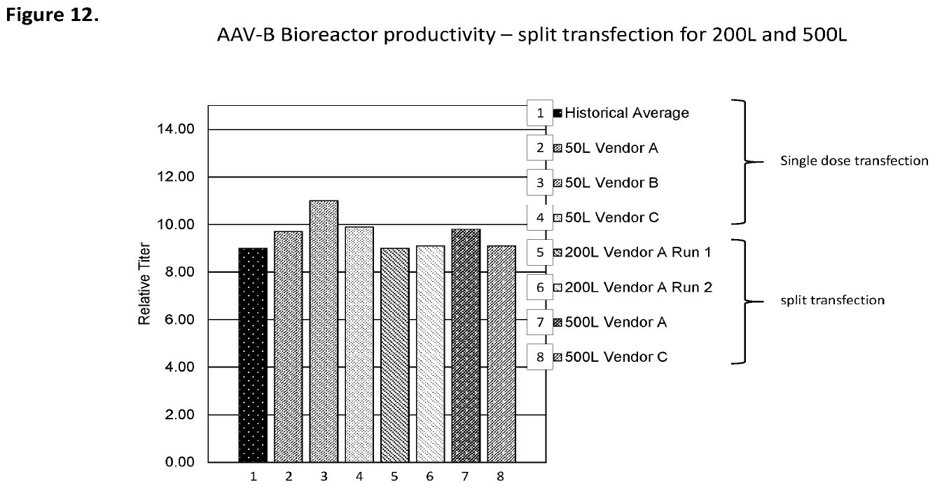

AAV-B productivity of a split transient transfection based process was tested in a 500 L bioreactor using a suspension adapted HEK293 clone. Transfection complex preparation process flow diagram for the split transfection based process is shown in FIG. 11. Two ˜21 L transfection complex doses were added to the culture with 15-30 minute between additions. The supernatant of the cultures was harvested at and ECD of 168 hours, i.e., 4 days post-transfection. Relative titer of AAV-B produced is shown in FIG. 12. 500 L bioreactor productivity was similar to both the “Historical Average” and to the productivity obtained using a single dose transfection process in 200 L and 50 L bioreactors.

### Example 6—AAV-B Productivity of Split Transient Transfection-Based Process in a 200 L Scale Down of a 2,000 L Process

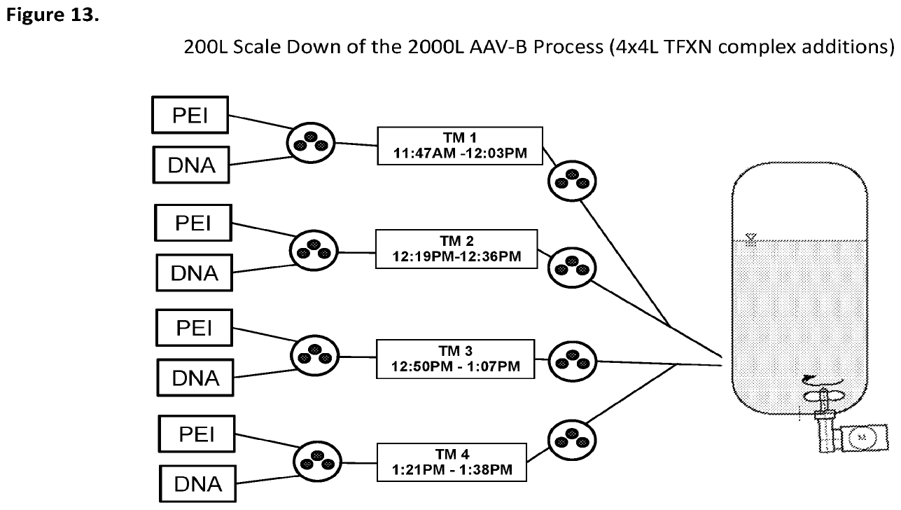

AAV-B productivity of a 200 L scale down of a 2,000 L split transient transfection based process was tested in a 200 L bioreactor. The seed train culture medium comprised an anti-clumping agent. Four ˜4 L transfection complex doses were added to the culture with ˜15 minute between additions. A process flow diagram is shown in FIG. 13. The supernatant of the cultures was harvested at and ECD of 168 hours, i.e., 4 days post-transfection. The titer of the supernatant was ˜6.25E+10. The lysed titer was ˜1.15E+11.

### Example 7—AAV Productivity of a 2,000 L Split Transient Transfection-Based Process

In some embodiments, a 2,000 L split transient transfection based process will comprise transferring 4×˜42 L transfection complexes to a 2,000 L bioreactor comprising ˜1600 L cell culture (e.g., HEK cell culture). In some embodiments, the culture will comprise an anti-clumping agent. In some embodiments, transfection complexes will be produced by mixing diluted one or more polynucleotides and diluted at least one transfection reagent using an inline mixer, wherein the mixing comprises transferring the diluted one or more polynucleotides and the diluted at least one transfection reagent from two separate containers into a new container at a rate of about 8 liters/min. In some embodiments, the transfection complexes will be held for 10 to 20 minutes (e.g., 10 to 15 minutes) prior to transfer to the bioreactor. In some embodiments, the transfection complexes will be transferred to the bioreactor at a rate of about 5 L/min. In some embodiments, each batch of transfection complexes will be transferred to the bioreactor in 30 minutes or less. In some embodiments, there will be a 10-20 minute (e.g., ˜15 minute) gap between finishing the transfer of one batch of transfection complexes and starting the transfer of the next batch of transfection complexes.

While the disclosed methods have been described in connection with what is presently considered to be the most practical and preferred embodiments, it is to be understood that the methods encompassed by the disclosure are not to be limited to the disclosed embodiments, but on the contrary, is intended to cover various modifications and equivalent arrangements included within the spirit and scope of the appended claims.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

1. A method of producing a recombinant virus particle, comprising
a) providing a suspension cell culture of between about 200 liters and about 20,000 liters comprising a population of cells capable of producing the recombinant virus particle;
b) admixing one or more polynucleotides with at least one transfection reagent to form a first mixture, incubating the mixture to form polynucleotide:transfection reagent complexes, and transferring the polynucleotide:transfection reagent complexes to the suspension cell culture to transfect the cells;
c) admixing the one or more polynucleotides with the at least one transfection reagent to form a second mixture, incubating the mixture to form polynucleotide:transfection reagent complexes, and transferring the polynucleotide:transfection reagent complexes to the suspension culture to transfect the cells; and
d) maintaining the suspension cell culture comprising the transfected cells under conditions that allow the production of the recombinant virus particle,
wherein
(i) the one or more polynucleotide contain genes necessary for producing the recombinant virus particle;
(ii) the combined volume of the polynucleotide:transfection reagent complexes transferred to the suspension cell culture is between about 7% and about 15% of the volume of the suspension cell culture of step a);
(iii) the admixing, incubating and transferring of step b) and step c) are each completed in less than about 60 minutes;
(iv) the transferring in step b) and step c) is performed over a time period that is no longer than about 6 hours; and
(v) the transferring of step b) and step c) are performed simultaneously or consecutively in any order.

2. The method of claim 1, wherein step c) is repeated
(a) one more time;
(b) one or more times; or
(c) 1, 2, 3, 4, 5, 6, 7 or 8 times.

3-5. (canceled)

6. The method of claim 1, wherein the transferring of step c) starts
(a) between about 5 and about 60 minutes after completing the transferring of step b); or
(b) no more than about 60 minutes after completing the transferring of step b).

7. (canceled)

8. A method of increasing the production of a recombinant virus particle, comprising
a) providing a suspension cell culture of between about 200 liters and about 20,000 liters comprising a population of cells capable of producing the recombinant virus particle;
b) admixing one or more polynucleotides with at least one transfection reagent to form a first mixture, incubating the mixture to form polynucleotide:transfection reagent complexes, and transferring the polynucleotide:transfection reagent complexes to the suspension cell culture to transfect the cells;
c) admixing the one or more polynucleotides with the at least one transfection reagent to form a second mixture, incubating the mixture to form polynucleotide:transfection reagent complexes, and transferring the polynucleotide:transfection reagent complexes to the suspension cell culture to transfect the cells; and
d) maintaining the suspension cell culture comprising the transfected cells under conditions that allow the production of the recombinant virus particle,
wherein
(i) the one or more polynucleotide contain genes necessary for producing the recombinant virus particle;
(ii) the combined volume of the polynucleotide:transfection reagent complexes of b) and c) transferred to the suspension cell culture is between about 7% and about 15% of the volume of the suspension cell culture of step a);
iii the admixing, incubating and transferring of step b) and step c) are each completed in less than about 60 minutes;
(iv) the transferring in step b) and step c) is performed over a time period that is no longer than about 6 hours; and
(v) the transferring of step b) and step c) are performed simultaneously or consecutively in any order.

9. The method of claim 8, wherein step c) is repeated one more time or more than one more time.

10-14. (canceled)

15. The method of claim 1 wherein the admixing one or more polynucleotides with at least one transfection reagent is performed by an inline mixer.

16. The method of claim 1 wherein the admixing, incubating and transferring of step b) and step c) are each completed in less than about 30 minutes or in less than about 35 minutes.

17. (canceled)

18. The method of claim 1 wherein the incubating of step b) and step c) are each for about 10 to about 20 minutes or for about 10 to about 15 minutes.

19. (canceled)

20. The method of claim 1 wherein the at least one transfection reagent comprises a stable cationic polymer.

21. The method of claim 1 wherein the at least one transfection reagent comprises PEI.

22. The method of claim 1, wherein the suspension cell culture has a volume
(a) between about 200 liters and about 5,000 liters;
(b) between about 200 liters and about 2,000 liters;
(c) between about 200 liters and about 1,000 liters;
(d) between about 200 liters and about 500 liters;
(e) of about 200 liters, about 300 liters, about 400 liters, about 500 liters, about 750 liters, about 1,000 liters, about 2,000 liters, about 3,000 liters, or about 5,000 liters;
(f) of about 500 liters;
(g) of about 1,000 liters; or
(h) of about 2,000 liters.

23-30. (canceled)

31. The method of claim 1, wherein the population of cells comprises a population of mammalian cells.

32. The method of claim 1, wherein the population of cells comprises a population of HEK293 cells, HEK derived cells, CHO cells, CHO derived cells, HeLa cells, SF-9 cells, BHK cells, Vero cells, and/or PerC6 cells.

33. (canceled)

34. The method of claim 1, wherein the suspension cell culture provided in step a) comprises between about 2×10E+6 and about 10E+7 viable cell/ml.

35. The method of claim 1, wherein the suspension cell culture is maintained under conditions that allow production of the recombinant virus particle
(a) for between about 2 days and about 10 days, between about 3 days and about 5 days, or between about 5 days and 14 days;
(b) for about 2 days, about 3 days, about 4 days, about 5 days, about 6 days, or about 7 days;
(c) for at least about 3 days;
(d) for about 3 days; or
(e) for about 4 days.

36-39. (canceled)

40. The method of claim 1 wherein the recombinant virus particle is a recombinant adeno-associated virus (rAAV) particle or a recombinant lentivirus particle.

41. (canceled)

42. The method of claim 1, wherein the recombinant virus particle is a recombinant adeno-associated virus (rAAV) particle comprising a capsid protein of the AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 serotype.

43-51. (canceled)

52. The method of claim 1, wherein the recombinant virus particle is a recombinant adeno-associated virus (rAAV) particle and the one or more polynucleotide encode
a) an rAAV genome to be packaged,
b) adenovirus helper genes necessary for packaging,
c) an AAV rep protein sufficient for packaging, and
d) an AAV cap proteins sufficient for packaging.

53. The method of claim 52, wherein the one or more polynucleotide comprises a polynucleotide encoding the rAAV genome, a polynucleotide encoding the AAV rep protein and the AAV cap proteins, and a polynucleotide encoding the adenovirus helper functions.

54. (canceled)

55. The method of claim 1, wherein the recombinant virus particle is a recombinant adeno-associated virus (rAAV) particle and the method further comprises recovering the rAAV particles.

56. (canceled)

57. The method of claim 1, wherein the recombinant virus particle is a recombinant adeno-associated virus (rAAV) particle and the cell culture produces at least about twice as many rAAV particles measured as GC/ml than a reference method comprising a single step of admixing, incubating and transferring the same volume of polynucleotide:transfection reagent complexes.

58. (canceled)
